(19) United States
(12) Patent Application Publication
(10) Pub. No.: US 2024/0317819 A1
(43) Pub. Date: Sep. 26, 2024

(54) CHIMERIC KLEBICINS

(71) Applicant: BACTOCLEAR HOLDINGS PTE. LTD, Singapore (SG)

(72) Inventors: Anisha AMBADY, Bangalore (IN); Chemira Biddappa APPAIAH, Bangalore (IN); Vivek Daniel PAUL, Bangalore (IN); R Sanjeev SARAVANAN, Bangalore (IN); Keerthana ANBALAGAN, Periyakulam (IN)

(21) Appl. No.: 18/579,038

(22) PCT Filed: Jul. 12, 2022
(86) PCT No.: PCT/IN2022/050631

§ 371 Date: Jan. 12, 2024

(30) Foreign Application Priority Data
Jul. 12, 2021 (IN) 202141031183

(51) Int. Cl. C07K 14/26; A61K 38/00; C12N 15/63

(52) U.S. Cl. CPC C07K 14/26; C12N 15/63; A61K 38/00; C07K 2319/10; C07K 2319/74

(57) ABSTRACT

Polypeptides are chimeric klebicins, which have one or more translocation domains, one or more receptor-binding domains, and one or more bactericidal domains. Each of the domains is derived from a naturally-occurring klebicin. The killing domain and at least one of the receptor-binding domains are derived from two different naturally-occurring klebicins. The chimeric klebicins achieve improved target cell range and bactericidal activity. The chimeric klebicins can be incorporated into compositions and kits, and can be used in methods of suppressing growth of Klebsiella pneumoniae.

### BACKGROUND OF THE INVENTION

Antibacterial proteins produced by bacteria to kill related bacterial species, bacteriocins present important means for bacteria to compete for environmental resources. A bacteriocin includes several distinct domains, responsible for translocation into its target bacteria, for binding to its receptor and for exerting its bactericidal activity. The receptor-binding domain and the translocation domain act first allow a bacteriocin to gain entry into susceptible bacterial cells by utilizing cell-surface receptors of target bacteria and translocation machinery. Upon being transported across the cell membrane, the killing domain of the bacteriocin then functions to cause the death of the target bacterial cells. On the other hand, the bacteriocin-producing bacteria are themselves protected against killing effect of such bacteriocins by virtue of the presence of certain immunity proteins that can neutralize the bacteriocins. Thus, target cell susceptibility is predicated in the fact that cell surface receptors and translocation proteins are present whereas cognate immunity proteins are absent. These factors together determine a narrow range of effectiveness for a naturally-occurring bacteriocin.

Klebicins are bacteriocins produced by Klebsiella spp. and specifically kill other susceptible members of the Klebsiella genus, such as Klebsiella pneumoniae, a drug-resistant pathogenic bacterial species known to cause severe infectious diseases and thus a therapeutic target in the treatment of these diseases. Since the therapeutic use of klebicins tends to suffer from the disadvantages of narrow target ranges within the Klebsiella genus, there exists a pressing need for developing new and improved klebicins with broader target cell ranges in order to effectively treat Klebsiella infections. The invention addresses this and other related needs by disclosing novel chimeric bacteriocins, which are characterized by the inclusion of multiple domains from multiple original klebicins, retaining activity of both individual klebicins, and are capable of utilizing multiple receptors for entry. These new chimeric klebicins also have a reduced frequency of resistance (FoR), and the levels are equal or better than the individual klebicin combination. Lastly, these chimeric klebicins are able to achieve increased effective range-their bactericidal activity can be exerted across the activity spectrum of the individual klebicins, from which the individual domains of klebicins are derived.

### BRIEF SUMMARY OF THE INVENTION

With the goal in mind to develop new bacteriocins with improved properties such as broad target bacterial cell range and enhanced anti-bacterial activities, the present inventors engineered a panel of chimeric klebicins employing different combinations of translocation domain, receptor-binding domain, and bactericidal domain (or “killing domain”) taken from different naturally-occurring klebicins to study their bactericidal efficacy against the bacteria of the Klebsiella genus, especially Klebsiella pneumoniae. As such, in a first aspect, this invention provides new chimeric klebicin polypeptides that are capable of suppressing the growth of the Klebsiella spp. bacteria, e.g., Klebsiella pneumoniae. The chimeric polypeptide comprises one or more translocation domains, one or more receptor-binding domains, and a killing domain, with each of these domains taken from a naturally-occurring klebicin, and the killing domain and at least one of the receptor-binding domains taken from two different naturally-occurring klebicins. In some embodiments, the polypeptide comprises, from its N-terminus to C-terminus, the translocation domain(s), the receptor-binding domain(s), and the killing domain. In some embodiments, there may be two receptor-binding domains and two translocation domains in a chimeric klebicin polypeptide, and the two receptor-binding domains and translocation domains are from two different naturally-occurring klebicins. In some embodiments, the chimeric polypeptide may further comprise a peptide linker, which may be either a flexible linker or a rigid linker, located between the receptor-binding domain (e.g., the one closest to the C-terminus of the polypeptide) and the killing domain. In some embodiments, the killing domain comprises a full-length naturally-occurring klebicin. In some embodiments, the translocation domain and one of the receptor-binding domains are derived from P628 or P764. In some embodiments, the killing domain comprises full length P764 or P801. In some embodiments, the two different receptor-binding domains are taken from P764 and P774. In some embodiments, the polypeptide consists of or consists essentially of the components named in the specific combinations in Table 3 of this disclosure (see column 3 “Domain details”). For example, the chimeric klebicin may comprise or consists of one of the amino acid sequences set forth in Table 3, e.g., SEQ ID NO:7, 33, 45, 47, 51, 57, 59, 61, 65, 67, 69, 71, 73, or 75. Also provided is a composition comprising the chimeric polypeptide of this invention and a physiologically acceptable excipient. In some cases, the composition is formulated for administration either systemically or locally, e.g., in a suitable form for injection or for inhalation or for local delivery.

This invention also provides the polynucleotide sequences encoding the chimeric polypeptides described above and herein, their corresponding expression cassettes, vectors, and host cells. In some embodiments, this invention provides a nucleic acid comprising a polynucleotide sequence encoding the polypeptide comprising or consisting of SEQ ID NO:8, 34, 46, 48, 52, 58, 60, 62, 66, 68, 70, 72, 74, or 76. In some embodiments, this invention provides an expression cassette comprising such a polynucleotide sequence, or a vector comprising the expression cassette, or a host cell comprising the expression cassette or the vector described above or herein.

In some embodiments, a method is provided for recombinantly producing the chimeric klebicin polypeptide described above and herein. The method comprising culturing the host cell comprising an expression cassette (such as a part of a vector) encoding such a polypeptide under conditions permitting the expression of the chimeric polypeptide encoded by the expression cassette or the vector.

In a second aspect, the present invention provides methods for suppressing growth of Klebsiella pneumoniae by applying an effective amount of the composition containing the chimeric klebicin of the present invention to a location where Klebsiella pneumoniae is present. In some embodiments, the composition is applied to a patient suffering from a Klebsiella pneumoniae infection by injection, such as a liquid form like a solution or emulsion or suspension for intravenous or intramuscular or subcutaneous injection, or by inhalation of an aerosolized or nebulized spray or mist etc., or by local delivery such as topical application, for example, the polypeptide is applied in the form of a paste, cream, lotion, ointment, spray, or as an incorporated part of a patch/bandage or wound dressing.

A related aspect of this invention is the use of the chimeric klebicin polypeptide described herein for suppression of Klebsiella pneumoniae growth or the production of a medicament for treating Klebsiella pneumoniae infection. In some embodiments, the polypeptide is present in a composition formulated for injection, or for inhalation, or for local application. In some embodiments, the polypeptide used in the claimed method may comprise or consist of SEQ ID NO:7; or the polypeptides used in the claimed method may comprise or consist of SEQ ID NO:33; or the polypeptides used in the claimed method may comprise or consist of SEQ ID NO:45; or the polypeptides used in the claimed method may comprise or consist of SEQ ID NO:47; or the polypeptides used in the claimed method may comprise or consist of SEQ ID NO:51; or the polypeptides used in the claimed method may comprise or consist of SEQ ID NO:57; or the polypeptides used in the claimed method may comprise or consist of SEQ ID NO:59 or the polypeptides used in the claimed method may comprise or consist of SEQ ID NO:61; or the polypeptides used in the claimed method may comprise or consist of SEQ ID NO:65; or the polypeptides used in the claimed method may comprise or consist of SEQ ID NO:67; or the polypeptides used in the claimed method may comprise or consist of SEQ ID NO:69; or the polypeptides used in the claimed method may comprise or consist of SEQ ID NO:71; or the polypeptides used in the claimed method may comprise or consist of SEQ ID NO:73; or the polypeptides used in the claimed method may comprise or consist of SEQ ID NO:75.

In a third aspect, the present invention provides a kit for suppressing growth of Klebsiella pneumoniae, comprising a first container containing a composition comprising an effective amount of the chimeric klebicin polypeptide described herein. In some cases, the composition is formulated for injection. In some cases, the composition is formulated for inhalation. In some cases, the composition is formulated for local delivery. In some embodiments, the kit may further comprise a manual providing instructions for a user of the kit. Typically, the kit includes a first container containing a composition comprising an effective amount of a polypeptide comprising or consisting of the amino acid sequence of SEQ ID NO:7, 33, 45, 47, 51, 57, 59, 61, 65, 67, 69, 71, 73, or 75.

### BRIEF DESCRIPTION OF THE DRAWINGS

FIG. 1. Domains in a bacteriocin.

FIG. 2. Workflow for generating chimeric klebicins.

FIG. 3. Details of P775 with domain architecture (FIG. 3A), protein expression profile (FIG. 3B), purified protein profile (FIG. 3C) and activity on K. pneumoniae ATCC13883 by lawn inhibition assay (FIG. 3D).

FIG. 4. Protocol for Frequency of Resistance (FoR) studies of klebicins.

FIG. 5. Activity of P775 on P628-resistant mutants of ATCC13883. Lawn inhibition assay on wild-type (WT) and resistant mutants (R) (FIG. 5A) and MIC (FIG. 5B).

FIG. 6. Details of P810 with domain architecture (FIG. 6A), protein expression profile (FIG. 6B) and activity on K. pneumoniae ATCC13883 by lawn inhibition assay (FIG. 6C).

FIG. 7. Details of P821 with domain architecture (FIG. 7A), purified protein profile (FIG. 7B) and activity on K. pneumoniae ATCC13883 by lawn inhibition assay (FIG. 7C).

FIG. 8. Bactericidal activity of P821 on K. pneumoniae strains ATCC13883 (FIG. 8A) and B2101 (FIG. 8B).

FIG. 9. Details of P823 with domain architecture (FIG. 9A), protein expression profile (FIG. 9B) and activity on K. pneumoniae ATCC13883 by lawn inhibition assay (FIG. 9C).

FIG. 10. Details of P835, P836, and P837 with domain architecture (FIG. 10A), purified protein profile (FIG. 10B) and activity on K. pneumoniae ATCC13883 by lawn inhibition assay (FIG. 10C). Bactericidal activity of P836 by CFU drop assay (FIG. 10D)

FIG. 11. Details of P845 and P862 with domain architecture (FIG. 11A), purified protein profile (FIG. 11B) and activity on K. pneumoniae ATCC13883 by lawn inhibition assay (FIG. 11C). Assessment of hybrid activity (FIG. 11D).

FIG. 12. Details of P863 with domain architecture (FIG. 12A), purified protein profile (FIG. 12B) lawn inhibition assay and assessment of hybrid activity (FIG. 12C).

FIG. 13. Details of P867 with domain architecture (FIG. 13A), purified protein profile (FIG. 13B). Activity by lawn inhibition assay (FIG. 13C).

FIG. 14. Details of P870 with domain architecture (FIG. 14A), purified protein profile (FIG. 14B). Activity by lawn inhibition assay (FIG. 14C).

FIG. 15. Details of P875 with domain architecture (FIG. 15A), purified protein profile (FIG. 15B). Activity by lawn inhibition assay (FIG. 15C).

FIG. 16. Details of P889 with domain architecture (FIG. 16A), purified protein profile (FIG. 16B). Activity by lawn inhibition assay (FIG. 16C).

FIG. 17. Details of P891 with domain architecture (FIG. 17A), purified protein profile (FIG. 17B). Activity by lawn inhibition assay (FIG. 17C).

FIG. 18. Details of P892 with domain architecture (FIG. 18A), purified protein profile (FIG. 18B). Activity by lawn inhibition assay (FIG. 18C).

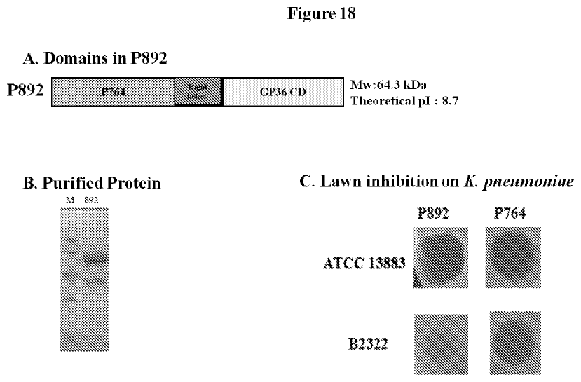

### DEFINITIONS

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “recombinant” when used with reference, e.g., to a cell, or a nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct transcription of a polynucleotide sequence. As used herein, a promoter includes necessary polynucleotide sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a polynucleotide expression control sequence (such as a promoter, or array of transcription factor binding sites) and a second polynucleotide sequence, wherein the expression control sequence directs transcription of the polynucleotide sequence corresponding to the second sequence.

An “expression cassette” is a nucleic acid construct, generated recombinantly or synthetically, with a series of specified polynucleotide elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

The term “translocation domain” as used in the context of describing a segment of a klebicin refers to the segment that is responsible for mediating a process by which the protein is introduced into or taken up by a target Klebsiella bacterial cell across the cell membrane.

The term “receptor-binding domain” as used in the context of describing a segment of a klebicin refers to the segment having the capability of binding one or more components (e.g., receptors) present on the target Klebsiella bacterial cell membrane.

The term “killing domain” as used in the context of describing a segment of a klebicin refers to the segment responsible for the cytotoxicity of the klebicin to the target Klebsiella bacterial cell. For example, such cytotoxicity may be a pore-forming capability in a bacterial cell membrane or may be a lipid II-cleaving activity analogous to the activity of E. coli colicin M (Gross and Braun, Mol. Gen. Genet. 1996, 251:388-396; Barreteau et al., Microbial. Drug Resist. 2012, 18:222-229; and El Ghachi et al., J. Biol. Chem. 2006, 281:22761-22772), or nuclease activity (Dnase or Rnase). When used in describing the structure of a particular chimeric klebicin of this invention, the term “killing domain” may broadly encompass a full-length naturally-occurring klebicin or a modified version thereof (e.g., deletion, insertion, and/or substitution of one or more amino acid at one or more locations within of a full-length wild-type klebicin) that retains the capability of killing the target bacterial cells.

The term “consisting essentially of,” or its grammatical variations, as used in the context of describing the components of a chimeric klebicin of this invention, describes the chimeric klebicin as containing only these specifically named components (such as the translocation, receptor-binding, and kill domains, as well as peptide linkers identified in column 3 “Domain details” in Table 3 of this disclosure), excluding other components of the same or similar nature, but permitting the optional presence of up to additional 1, 2, 3, 4, 5, 6, 7, 8, 9, or up to 10 amino acids at the juncture between any two of the specified components of the chimeric klebicin as well as up to additional 1, 2, 3, 4, 5, 6, 7, 8, 9, 10, 11, 12, 13, 14, 15, 16, 17, 18, 19, or 20 amino acids at the N-terminus and/or the C-terminus.

The term “heterologous” as used in the context of describing the relative location of two elements, refers to the two elements such as polynucleotide sequences (e.g., a promoter or a protein/polypeptide-encoding sequence) or polypeptide sequences (e.g., a chimeric klebicin sequence selected from SEQ ID NOs:7, 33, 45, 47, 51, 57, 59, 61, 65, 67, 69, 71, 73, and 75 or another peptide sequence serving as a fusion partner with a chimeric klebicin sequence) that are not naturally found in the same relative positions. Thus, a “heterologous promoter” of a gene refers to a promoter that is not naturally operably linked to that gene. Similarly, a “heterologous polypeptide” or “heterologous polynucleotide” to a chimeric klebicin or its encoding sequence is one derived from an origin other than any of the naturally-occurring klebicins contributing any one of the translocation, receptor-binding, and kill domains, or is one derived from such a naturally-occurring klebicin but not naturally connected to any part of chimeric klebicin in the same fashion as found in nature. The fusion of a chimeric klebicin (or its coding sequence) with a heterologous polypeptide (or polynucleotide sequence) should typically result in a longer polypeptide (or polynucleotide sequence) retaining the same biological activity, for example, cytotoxicity towards the same targeted bacterial species.

The term “inhibit/inhibiting/inhibition” or “suppress/suppressing/suppression,” as used herein, refers to any detectable negative effect on a target biological process, such as bacterial cell proliferation or bacterial cell presence. Typically, an inhibition is reflected in a decrease of at least 10%, 20%, 30%, 40%, or 50% in target process (e.g., the growth rate or level of a pertinent bacterium such as K. pneumoniae) upon application of an inhibitory substance (e.g., any one of the chimeric klebicins set forth in SEQ ID NO:7, 33, 45, 47, 51, 57, 59, 61, 65, 67, 69, 71, 73, or 75), when compared to a control where the inhibitor is not applied.

The term “treat” or “treating,” as used in this application, describes to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “effective amount” as used herein refers to an amount of a given substance that is sufficient in quantity to produce a desired effect. For example, an effective amount of a chimeric klebicin for suppressing the growth of a specific bacterial species such as K. pneumoniae is the amount of the chimeric protein to achieve a decreased level (including to an undetectable level) of K. pneumoniae in a sample taken from a recipient, who is given the chimeric enzyme for a condition involving the bacterium's presence, e.g., as reflected or measured in the type of samples from the recipient. An amount adequate to achieve an intended effect in the therapeutic context is defined as the “therapeutically effective dose.” The dosing range varies with the nature of the therapeutic agent being administered and other factors such as the route of administration and the severity of a patient's condition.

As used herein, a “host cell” is a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

As used herein, the term “about” denotes a range encompassing +/−10% of a pre-determined value. For example, “about 10” means a range of 9 to 11.

### DETAILED DESCRIPTION OF THE INVENTION

### I. Introduction

Previously, polypeptides of bacterial origins possessing the activity of suppressing bacterial growth, especially for the genus of Klebsiella, by way of lysis of targeted bacteria cell wall and therefore killing the bacteria have been disclosed, see, e.g., WO2020/245376. These naturally-occurring polypeptides generally comprise the translocation domain (TD), receptor-binding domain (RD), and cytotoxicity domain (or killing domain, KD) as the main structural components.

The present inventors have constructed and identified chimeric polypeptides having the same general structural features by mixing and matching the translocation domain, receptor-binding domain, and cytotoxic domain (or “killing domain”) taken from different naturally-occurring klebicins, which possess highly unique and exceptionally valuable characteristics of bactericidal activity against certain target bacteria species within the genus of Klebsiella, especially K. pneumoniae. This disclosure relates to the compositions and methods of use based on these newly constructed and identified chimeric polypeptides with desirable anti-bacterial activity.

### II. Production of Chimeric Polypeptides

### A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a polynucleotide encoding a chimeric polypeptide and its variants can be verified after cloning or subcloning using, e.g., the chain termination method for sequencing double-stranded templates of Wallace et al., Gene 16: 21-26 (1981).

### B. Cloning and Subcloning of Coding Sequences for Chimeric Polypeptides

Polynucleotide sequences encoding chimeric klebicin polypeptides can be determined based on their amino acid sequences (e.g., any one of SEQ ID NOs:7, 33, 45, 47, 51, 57, 59, 61, 65, 67, 69, 71, 73, and 75) and available information from earlier publications (e.g., WO2020/245376).

Upon acquiring a polynucleotide sequence encoding a chimeric polypeptide, the coding sequence can be modified as appropriate (e.g., adding a coding sequence for a heterologous tag, such as an affinity tag, for example, 6×His tag or GST tag; or further mutated) and then be subcloned into a vector, for instance, an expression vector, so that a recombinant chimeric polypeptide can be produced from the resulting construct, for example, after transformation and culturing host cells under conditions permitting recombinant protein expression directed by a promoter operably linked to the coding sequence.

### C. Modification of Nucleic Acids for Preferred Codon Usage in a Host Organism

The polynucleotide sequence encoding a chimeric klebicin polypeptide can be further altered to coincide with the preferred codon usage of a particular host. For example, the preferred codon usage of one strain of bacterial cells can be used to derive a polynucleotide that encodes a chimeric klebicin of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of the chimeric klebicin polypeptides.

### IV. Expression and Purification of Recombinantly Produced Polypeptides

Following verification of the coding sequence, the chimeric polypeptides of the present invention can be produced using routine techniques in the field of recombinant genetics, relying on the polynucleotide sequences encoding the polypeptides disclosed herein.

### A. Expression Systems

To obtain high level expression of a nucleic acid encoding a chimeric klebicin polypeptide of the present invention, one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Sambrook and Russell, and Ausubel et al., Bacterial expression systems for expressing a recombinant polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the chimeric polypeptide in host cells. A typical expression cassette thus contains a promoter operably linked to the coding sequence and signals required for efficient polyadenylation of the transcript, ribosome binding sites, and translation termination. The nucleic acid sequence encoding the chimeric polypeptide may be linked to a cleavable signal peptide sequence to promote secretion of the recombinant polypeptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., His or c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMT010/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the chimeric polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant protein (e.g., a chimeric klebicin polypeptide of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

### B. Transfection Methods

Standard transfection methods are used to produce bacterial, mammalian, yeast, insect, or plant cell lines that express large quantities of a recombinant polypeptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium chloride transformation, calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

### C. Detection of Recombinant Expression of Chimeric Klebicin in Host Cells

After the expression vector is introduced into appropriate host cells, the transfected cells are cultured under conditions favoring expression of the chimeric klebicin polypeptide. The cells are then screened for the expression of the recombinant polypeptide, which is subsequently recovered from the culture using standard techniques (see, e.g., Scopes, Protein Purification: Principles and Practice (1982); U.S. Pat. No. 4,673,641; Ausubel et al., supra; and Sambrook and Russell, supra).

Several general methods for screening gene expression are well known among those skilled in the art. First, gene expression can be detected at the nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot). The presence of nucleic acid encoding a chimeric klebicin polypeptide in transfected cells can also be detected by PCR or RT-PCR using sequence-specific primers.

Second, gene expression can be detected at the polypeptide level. Various immunological assays are routinely used by those skilled in the art to measure the level of a gene product, particularly using polyclonal or monoclonal antibodies that react specifically with a chimeric klebicin of the present invention (e.g., Harlow and Lane, Antibodies, A Laboratory Manual, Chapter 14, Cold Spring Harbor, 1988; Kohler and Milstein, Nature, 256: 495-497 (1975)). Such techniques require antibody preparation by selecting antibodies with high specificity against the chimeric klebicin. The methods of raising polyclonal and monoclonal antibodies are well established and their descriptions can be found in the literature, see, e.g., Harlow and Lane, supra; Kohler and Milstein, Eur. J. Immunol., 6: 511-519 (1976).

### D. Purification of Recombinantly Produced Chimeric Klebicins

Once the expression of a recombinant chimeric klebicin polypeptide in transformed or transfected host cells is confirmed, the host cells are then cultured in an appropriate scale for the purpose of purifying the recombinant polypeptide.

1. Purification of Recombinantly Produced Polypeptides from Bacteria

When the chimeric klebicin polypeptides of the present invention are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, NY). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides, e.g., a chimeric klebicin polypeptide, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### 2. Standard Protein Separation Techniques for Purification

When a recombinant polypeptide, e.g., a chimeric polypeptide of the present invention, is expressed in host cells in a soluble form, its purification can follow the standard protein purification procedure described below.

i. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

ii. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a chimeric polypeptide of the present invention. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

iii. Column Chromatography

The proteins of interest (such as a chimeric polypeptide of the present invention) can also be separated from other proteins on the basis of their size, net surface charge, hydrophobicity, or affinity for ligands. In addition, antibodies raised against a chimeric klebicin can be conjugated to column matrices and the chimeric klebicin polypeptide immunopurified. All of these methods are well known in the art.

It will be apparent to one of skill that chromatographic techniques can be performed at any scale and using equipment from many different manufacturers (e.g., Pharmacia Biotech).

### V. Formulation and Administration

Various applications of the species-specific enzymatic activities can be immediately recognized. One important application is as antibacterial treatment of articles which may be contaminated in normal use. Locations, equipment, environments, or the like where target bacteria may be public health hazards may be treated using such entities. Locations of interest include public health facilities where the purpose or opportunity exists to deal with target bacteria containing materials. These materials may include waste products, e.g., liquid, solid, or air. Aqueous waste treatment plants may incorporate such to eliminate the target from effluent, whether by treatment with the enzyme entities directly, or by release of cells which produce such. Solid waste sites may introduce such to minimize possibility of target host outbreaks. Conversely, food preparation areas or equipment need to be regularly cleaned, and the invention provides compositions and means to effectively eliminate target bacteria. Medical and other public environments subject to contamination may warrant similar means to minimize growth and spread of target microorganisms. The methods may be used in contexts where sterilization elimination of target bacteria is desired, including air filtration systems for an intensive care unit.

Alternative applications include use in a veterinary or medical context. Means to determine the presence of particular bacteria, or to identify specific targets may utilize the effect of selective agents on the population or culture. Inclusion of bacteriostatic or bactericidal activities to cleaning agents, including washing of animals and pets, may be desired.

The chimeric klebicin polypeptides of this invention can be used to treat infections caused by specific, harmful bacterial species in, e.g., humans or animals, for conditions such as pneumonia, bacteremia, or urinary tract infection. These chimeric polypeptides can be administered prophylactically or can be administered to a subject that has contracted a bacterial infection. In one embodiment, the chimeric polypeptides are used to treat infections (e.g., respiratory infections) caused by one or more bacteria of a Klebsiella species, such as K. pneumoniae, and in case of respiratory viral infections to prevent the onset of bacterial secondary infections caused by Klebsiella species.

In one embodiment, these chimeric proteins (e.g., any one of those in Table 3, such as SEQ ID NOs:7, 33, 45, 47, 51, 57, 59, 61, 65, 67, 69, 71, 73, and 75) are used to treat humans or other animals that are infected with K. pneumoniae.

The route of administration and dosage will vary with the infecting bacteria strain(s), the site and extent of infection (e.g., local or systemic), and the subject being treated. The routes of administration include but are not limited to: oral, aerosol or other device for delivery to the lungs, nasal spray, intravenous (IV), intramuscular, intraperitoneal, intrathecal, intraocular, subcutaneous, vaginal, rectal, topical, lumbar puncture, intrathecal, and direct application to the brain and/or meninges. Excipients that can be used as a vehicle for the delivery of the therapeutic will be apparent to those skilled in the art. For example, the chimeric klebicin could be kept in a lyophilized form and be dissolved just prior to administration by IV injection. The dosage of administration is contemplated to be in the range of about 0.03, 0.1, 0.3, 1, 3, 10, 30, 100, 300, 1000, 3000, 10000 or more klebicin molecules per bacterium in the host infection. Depending upon the size of the klebicin, which may itself be tandemly associated, or in multiple subunit form (dimer, trimer, tetramer, pentamer, and the like) or in combination with one or more other entities, e.g., enzymes or fragments of different specificity, the dose may be about 1 million to about 10 trillion/per kg/per day, and preferably about 1 trillion/per kg/per day.

The therapeutic compositions comprising at least one of the chimeric klebicins of this invention are typically administered until successful elimination of the target pathogenic bacteria is achieved. Thus the invention contemplates single dosage forms, as well as multiple dosage forms of the compositions of the invention, as well as methods for accomplishing sustained release means for delivery of such single and multi-dosages forms.

With respect to the aerosol administration to the lungs or other mucosal surfaces, the therapeutic composition is incorporated into an aerosol formulation specifically designed for administration. Many such aerosols are known in the art, and the present invention is not limited to any particular formulation. An example of such an aerosol is the Proventil inhaler manufactured by Schering-Plough, the propellant of which contains trichloromonofluoro-methane, dichlorodifluoromethane, and oleic acid. Other embodiments include inhalers that are designed for administration to nasal and sinus passages of a subject or patient. The concentrations of the propellant ingredients and emulsifiers are adjusted if necessary based on the specific composition being used in the treatment. Also useful are suppositories that are designed for direct deposit of a klebicin-containing composition to an anatomic site of a bacterial infection, e.g., urinary tract.

Methods to evaluate killing capacity of the chimeric klebicins of this invention are often similar to many methods used in assessing killing capacity of intact replicating phages. Comparing total bacterial counts with viable colony units can establish what fraction of bacteria are actually viable, and by implication, what fraction have been susceptible to the killing constructs. Other means for evaluating stasis activity may include release of intracellular contents, whether natural or loaded, or enzymatic activity on defined or prepared substrates which correspond to natural cell wall structures.

Typically, the killing will decrease bacterial replication capacity to about ⅓ or less, and may affect or reduce it to about 1/10, 1/30, 1/100, 1/300, etc., to many orders of magnitude when compared to a control without exposure to a chimeric klebicin. However, even slowing the rate of bacterial replication without killing may have significant therapeutic or commercial value. Preferred genetic inactivation efficiencies may be 0.1, 0.2, 0.3, 0.5, 0.8, 1, 1.5, 2.0, 2.5, 3.0, 3.5, 4, 5, 6, 7, 8, or more log units.

This invention further contemplates pharmaceutical compositions comprising at least one chimeric klebicins described herein and a physiologically or pharmaceutically acceptable excipient. The compositions of the invention thus include formulations comprising an isolated chimeric polypeptide specifically targeting one or more bacteria of a genus such as the Klebsiella genus, e.g., K. pneumoniae. In some cases, a mixture of two, three, or four of the chimeric polypeptides may be used to enhance the target-specific bacteria killing without significant impact on other non-target bacterial species that may provide potential benefits to a patient. In this manner, the compositions of the invention can be tailored to the needs of the patient. The compounds or compositions will typically be sterile or near sterile.

By “therapeutically effective dose” herein is meant a dose that produces effects, bacteriostatic or preferably bactericidal, for which it is administered. The exact dose will depend on the purpose of the treatment, and will be ascertainable by one skilled in the art using known techniques. See, e.g., Ansel, et al. Pharmaceutical Dosage Forms and Drug Delivery; Lieberman (1992) Pharmaceutical Dosage Forms (vols. 1-3), Dekker, ISBN 0824770846, 082476918X, 0824712692, 0824716981; Lloyd (1999) The Art, Science and Technology of Pharmaceutical Compounding; and Pickar (1999) Dosage Calculations. As is known in the art, adjustments for protein degradation, systemic versus localized delivery, and rate of new protease synthesis, as well as the age, body weight, general health, sex, diet, time of administration, drug interaction, spectrum of bacterial components in the colony, and the severity of the condition may be necessary, and will be ascertainable with some experimentation by those skilled in the art.

Various physiologically or pharmaceutically acceptable excipients are well known in the art. As used herein, “physiologically or pharmaceutically acceptable excipient” refers to a material that, when combined with an active ingredient of a composition, allows the ingredient to retain its biological activity and without causing any detectable physiological reactions in a recipient. Such excipients may include stabilizers, preservatives, salt, or sugar complexes or crystals, and the like.

Exemplary pharmaceutically carriers include sterile aqueous of non-aqueous solutions, suspensions, and emulsions. Examples include, but are not limited to, standard pharmaceutical excipients such as a phosphate buffered saline solution, water, emulsions such as oil/water emulsion, and various types of wetting agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In other embodiments, the compositions will be incorporated into solid matrix, including slow release particles, glass beads, bandages, inserts on the eye, and topical forms such as creams, pastes, lotions, ointments, liquids or semi-liquids including solutions or suspensions, or incorporated into patches, bandages, or any type of wound dressings.

A composition comprising a chimeric enzyme of the invention may also be lyophilized using means well known in the art, e.g., for subsequent reconstitution and use according to the invention.

Also of interest are formulations for liposomal delivery, and formulations comprising microencapsulated enzymes, including sugar crystals. Compositions comprising such excipients are formulated by well-known conventional methods (see, e.g., Remington's Pharmaceutical Sciences, Chapter 43, 14th Ed., Mack Publishing Col, Easton PA 18042, USA).

In general, pharmaceutical compositions can be prepared in various forms, such as granules, tablets, pills, suppositories, capsules (e.g., adapted for oral delivery), microbeads, microspheres, liposomes, suspensions, salves, lotions, and the like. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Formulations may incorporate stabilizing agents, wetting and emulsifying agents, salts for varying the osmotic pressure or buffers for securing an adequate pH value.

### VI. Kits

The invention also provides kits for selectively suppressing the growth of certain targeted bacterial species, especially in the presence of other non-targeted bacterial species that might be closely related to the targeted bacterial species (e.g., belonging to the same genus) according to the method of the present invention. The kits typically include a first container that contains a composition including an effective amount of a polypeptide of interest, e.g., one in Table 3 such as one comprising or consisting essentially of an amino acid sequence selected from SEQ ID NOs:7, 33, 45, 47, 51, 57, 59, 61, 65, 67, 69, 71, 73, and 75. The polypeptide is typically present in a composition formulated for systemic delivery, for example, in the form of a solution (e.g., aqueous solution) or dispersion suitable for injection by intravenous, intramuscular, or subcutaneous means or in the form of a pill, tablet, caplet, or capsule suitable for oral ingestion, or for direct delivery via respiratory system, for example, in the form of a spray or aerosolized mist suitable for inhalation into the respiratory airway, or for local delivery, for example, in the form of a cream, paste, lotion, ointment, spray and the like suitable for topical application to a surface such as skin, or being incorporated into a skin patch, a bandage, or wound dressing for use on a patient's skin.

Optionally, the kit may further include informational material such as instructions for a user on how to use the kit for suppressing the growth of certain targeted bacterial species.

### Examples

The following examples are provided by way of illustration only and not by way of limitation. Those of skill in the art will readily recognize a variety of non-critical parameters that could be changed or modified to yield essentially the same or similar results.

### Introduction

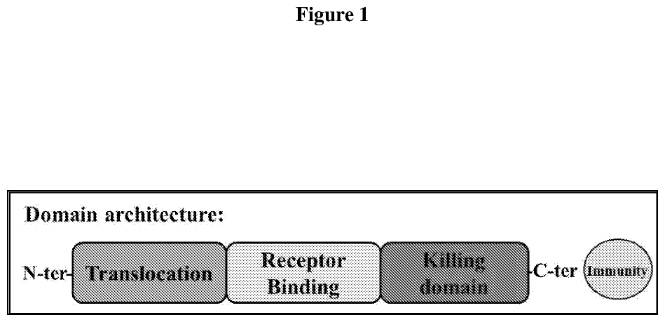

Bacteriocins are protein antibacterials produced by bacteria to kill similar or closely related species, as a survival mechanism in competing ecological niches. Bacteriocins parasitize on components of bacterial machinery involved in transport of essential nutrients and minerals such as iron and vitamin B₁₂ for cell entry. Bacteriocin-producing bacteria are themselves protected from lethal activity by the presence of immunity proteins that bind to the bacteriocin and sterically prevent its activity. Bacteriocins are be highly genus specific and target the same genus as the producer organism. Large bacteriocins (>30 kDa) are modular proteins and many of them are natural chimeric proteins, evolved from varied sources having receptor binding, translocation and killing domain, functioning as a single unit to kill susceptible bacteria (FIG. 1). Using the receptor binding and translocation domains, bacteriocins enter bacterial cells by parasitizing on cell-surface receptors Bacteriocins after binding to their cognate receptors at the cell surface and are transported through the outer membrane of susceptible bacteria by using either the Tol system (Group A) or the Ton system (Group B). After entry into the cell, the killing domain can effect bactericidal action, through multiple modes such as by membrane damage, or by interfering in metabolic pathways, and DNase or RNase activities.

Susceptibility to bacteriocins is primarily driven by the presence of cell surface receptor and translocation proteins expressed by susceptible bacteria, and to a lesser extent by the absence of a cognate immunity protein. Hence most bacteriocins exhibit a narrow coverage. Although bacteria can remain sensitive to a given bacteriocin even when the immunity is present, bacteria lacking its cognate receptor will be completely insensitive to the bacteriocin. Bacteriocins utilize only a single cell surface receptor and a single translocation system for cell entry in to susceptible bacteria. Any mutation(s) in genes involved in either the receptor or translocation machinery will render the bacteria resistant to bacteriocins adding to the restricted coverage. Under laboratory conditions, many bacteriocins studied so far demonstrate an in vitro frequency of resistance (FoR) of 10⁻⁶ to 10⁻⁷. Due to the dual reason of narrow coverage and higher FoR, bacteriocins were not previously considered as a therapeutic option for treating bacterial infections.

### Bacteriocins of Klebsiella spp.: Klebicins

Klebsiella pneumoniae is a highly drug-resistant pathogenic bacteria. In humans, it causes serious infections including pneumonia, bacteremia and urinary tract infections. Due to high burden of antimicrobial resistance, new interventions are required for treatment of infections caused by these bacteria.

Klebicins are large molecular weight bacteriocins produced by Klebsiella spp. and specifically kill other susceptible members of the Klebsiella genus including the pathogenic Klebsiella pneumoniae with possible utility as a therapeutic option. However, as known for other bacteriocins, klebicins also have properties such as narrow coverage within the Klebsiella and high FoR. Since the clinical utility of klebicins are yet to be explored, pre-existing resistance may be very minimal and emergence of resistance will be only limited to the susceptible bacteria.

Improving properties of Klebicins

### 1. Cocktails

The properties of narrow coverage, FoR could be overcome using a combination of klebicins that recognize different cell surface receptors, different mechanism of action (MoA) and contain both group A and B klebicins as individual components in the cocktail. This will enhance the utility of klebicins as therapeutic entities. Klebicins and their receptor (RD), translocation (TD) and killing domains (KD) are listed in Table 1.

TABLE 1
Natural klebicins

| Protein | Homolog of | | Mechansim of | Mechanism of | |
|---|---|---|---|---|---|
| ID | Klebicins Type | Receptor | Translocation | cytotoxicity | Mw/PI |
| P611 | Klebicin B | Not known | Not known | DNase | 79.6/8.96 |
| P628 | Klebicin CCL | IutA | Tol | RNase | 59.2/9.21 |
| P764 | Kpne A | OmpC | Ton | Pore forming | 39.8/8.96 |
| P774 | KpneM | FhuA | Ton | Peptidoglycan | 30.2/5.57 |
| | | | | synthesis inhibitor | |
| P801 | KvarM | FhuA | Ton | Peptidoglycan | 29.8/8.9 |
| | | | | synthesis inhibitor | |

The FoR of these klebicins were studied by plating high concentrations (>10⁹ cells) of K. pneumoniae strain ATCC 13383 on agar plates containing 8×MIC concentrations of individual and combinations containing two klebicins. Colonies that were recovered from these plates were enumerated to determine the recovery frequency which will correspond to the highest possible FoR (Table 2). With single klebicins, the FoR was in the range of 10⁻⁶ to 10-7 indicating possible emergence of resistance during therapy. However, when the plating was done on a combination of two klebicins, all combinations except P774 and P801 reduced FoR to <10⁻⁹ demonstrating that the appropriate klebicin combinations can mitigate emergence of resistance. The combination of P774 and P801 did not reduce FoR possibly due to the identical MoA—both P774 and P801 recognize the same cell surface receptor and utilize TonB pathway for translocation. Hence, combination of klebicins with different MoA in a cocktail will aid in mitigating the FoR.

TABLE 2
Frequency of resistance (FoR) of natural klebicins
individually and in combinations

| Klebicins and | Recovery |
|---|---|
| combinations @ 8X | Frequency |
| P628 | ~1 × 10⁻⁷ |
| P764 | ~1 × 10⁻⁷ |
| P774 | ~1 × 10⁻⁶ |
| P801 | ~5 × 10⁻⁷ |
| P628 + P764 | <10⁻⁹ |
| P628 + P774 | <10⁻⁹ |
| P774 + P764 | <10⁻⁹ |
| P801 + P628 | <10⁻⁹ |
| P801 + P764 | <10⁻⁹ |
| P801 + P774 | ~5 × 10⁻⁷ |

Although the combination approach will not only help mitigate the resistance problem but also improve overall coverage of klebicins, there are few limitations for a cocktail approach. This includes increased cost of goods due to production and processing of multiple proteins, finding compatible storage buffers and conditions, challenges in formulation and establishing quality control parameters for multiple proteins.

To overcome these challenges, a novel strategy is to combine the properties of multiple klebicins into a single klebicin by protein engineering. This can be achieved by fusing or swapping domains from different klebicins to generate chimeric fusions with desired properties. Klebicins, like other bacteriocins, are multidomain protein structures that have evolved through natural evolution to recognize and kill susceptible bacteria. These are multidomain protein structures that fold into active confirmation and function as a single unit, there is no available information on whether generic protein engineering strategies such as domain swapping or domain additions will yield active proteins. Therefore, the combinations of domains or strategies that would work cannot be predicted a priori.

### 2. Chimeric Fusions

Chimeric klebicins with properties of combination of multiple klebicins will be generated empirically. The expected outcome is to have chimeric klebicins with improved coverage and FoR, as observed with a combination. Since the coverage of natural bacteriocins are limited by the single cell surface receptor recognition, the chimeric fusion strategy will be to design and engineer klebicins that can recognize multiple cell surface receptors and/or utilize both Tol and Ton pathways for translocation.

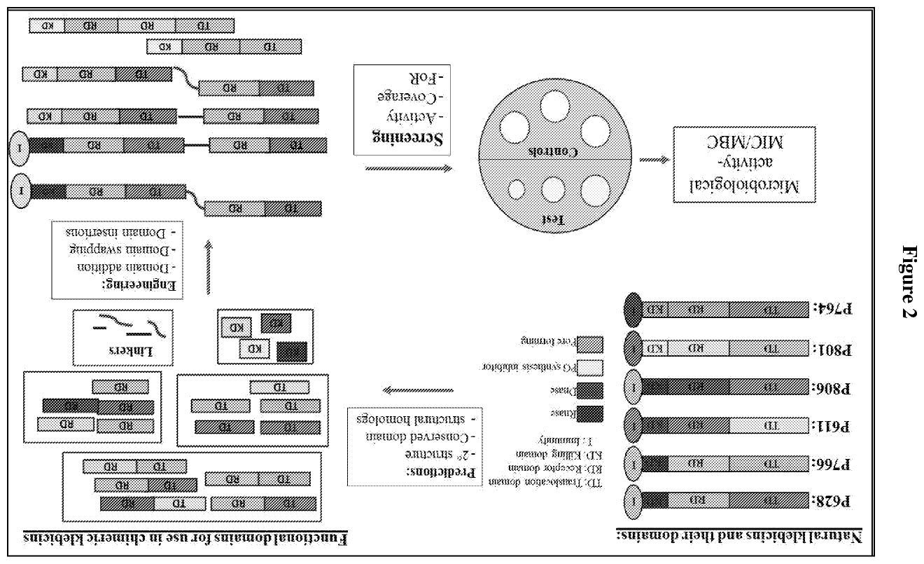

Strategies included adding the translocation domain (TD) and receptor binding domain (RD) of a klebicin, alone or together to another klebicin, inserting a RD from one klebicin into another klebicin to generate a twin RD, replacing the RD or KD of one klebicin with another one and fusing two klebicins together to make a single protein. Chimeric fusion workflow is depicted in FIG. 2.

Several chimeric klebicins were generated with domains from different klebicins fused together. Some of the chimeric fusions were active and showed antibacterial activity on a lawn of K. pneumoniae strain ATCC13883 (Table 3).

TABLE 3
Engineered klebicins generated by domain shuffling/domain additions;
FL: Full Length, TD: Translocation domain, RD: Receptor binding
domain, KD: Killing domain, CD: Catalytic domain

| | | | Antibacterial |
|---|---|---|---|
| | Pro- | | activity on |
| Sl. | tein | Domain details | K. pneumoniae |
| 1 | P771 | P764 TD RD + P628 FL | Yes |
| 2 | P772 | P764 TD RD- Flexible linker + P628 FL | Yes |
| 3 | P777 | P764 TD RD- Rigid linker + P628 FL | Yes |
| 4 | P775 | P628 TD RD- Rigid linker + P764 FL | Yes |
| 5 | P776 | P628 TD RD- Flexible linker + P764 FL | Yes |
| 6 | P780 | P774 TD RD- Rigid linker + P628 FL | Yes |
| 7 | P781 | P628 TD RD- Flexible linker + P774 FL | No |
| 8 | P782 | P774 TD RD- Flexible linker + P628 FL | Yes |
| 9 | P784 | P628 TD RD- Rigid linker + P774 FL | No |
| 10 | P787 | P764 TD RD- Flexible linker + P774 FL | No |
| 11 | P789 | P764 TD RD- Rigid linker + P774 FL | No |
| 12 | P788 | P764 TD - Rigid linker + P628 FL | Yes |
| 13 | P792 | P764- Rigid Linker + P774 FL | Yes |
| 14 | P795 | P764- Flexible Linker + P774 FL | Yes |
| 15 | P805 | P628 Δ7 | Yes |
| 16 | P806 | P628 Δ25 | Yes |
| 17 | P810 | P764 TD RD-P774 RD-P764 KD (Twin RD) | Yes |
| 18 | P812 | P628 TD RD-Rigid Linker-P764 KD | Yes |
| 19 | P818 | P764 TD-P774 RD + P764 KD | No |
| 20 | P819 | P774 TD- P764 RD + P774 KD | No |
| 21 | P820 | P764 TD RD- Rigid Linker- P628 425 | Yes |
| 22 | P822 | P801 TD RD-Rigid Linker + P764 KD | No |
| 23 | P821 | P628 TD RD-Rigid Linker + P801 FL | Yes |
| 24 | P823 | P628 TD RD-Flexible Linker + P801 FL | Yes |
| 25 | P835 | P628 TD RD + P801 FL | Yes |
| 26 | P836 | P628 TD RD-4X Rigid Linker + P801 FL | Yes |
| 27 | P837 | P628 TD RD-2X Flexible Linker + P801 FL | Yes |
| 28 | P842 | P628 FL + P801 FL | Yes |
| 29 | P845 | P628 FL- Flexible Linker + P801 FL | Yes |
| 30 | P862 | P628 FL- Rigid Linker + P801 FL | Yes |
| 31 | P863 | P764 FL- Rigid Linker + P801 FL | Yes |
| 32 | P864 | P764 FL- Flexible Linker + P801 FL | Yes |
| 33 | P870 | P764 FL- 4X Rigid Linker + P801 FL | Yes |
| 34 | P867 | 1-53 amino acids of P849 (P801 homolog | Yes |
| | | from K. quasipneumoniae) replaced with | |
| | | 1-22 amino acids of P801 | |
| 35 | P875 | P801-Rigid Linker + GP36CD | Yes |
| 36 | P889 | P764-GP36CD | Yes |
| 37 | P891 | P764-Flexible Linker + GP36CD | Yes |
| 38 | P892 | P764-TDRD-Rigid Linker + GP36 CD | Yes |

Note:
Constructs #1, 2, 3, 6, 8, 12, 15 and 16 listed in Table 3 are expressed along with the immunity protein of P628.

31 of 38 engineered klebicins demonstrated antibacterial activity proving that protein engineering strategies can be applied to generate active chimeric klebicins. It is also noticeable that a few of the chimeric molecules lost activity, indicating that not all engineered klebicins retain activity.

### Example 1: P775 (P628 TD RD-Rigid Linker+P764 FL)

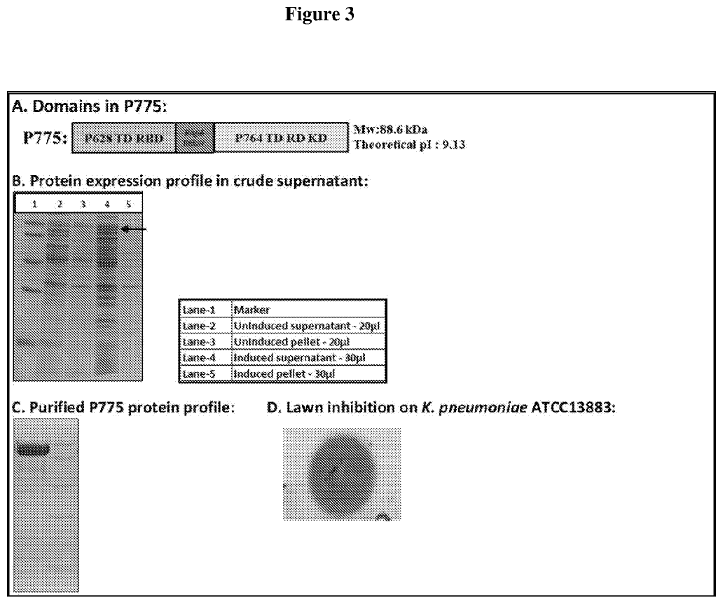

P775 was generated by fusing the TD and RD region of P628 with full length (FL) P764 with the aid of a rigid linker. This construct will have two translocation and receptor binding units that can recognize two different cell surface receptors and translocation machinery. P628 TD RD region and P764 were PCR amplified separately and fused together by synthesis by overlap extension (SOE) PCR (FIG. 3A). The fusion gene was cloned into pET26b expression vector, sequence confirmed and protein expression done in E. coli expression strain ER2566 (FIG. 3B). The expressed protein was purified by a two-step ion-exchange chromatography to obtain ˜90% homogenous protein (FIG. 3C) and 2-5 μg of the purified protein was active on K. pneumoniae ATCC13883 lawn (FIG. 3D). The MIC of P775 was 5 μg/mL on ATCC13883 in iron-deficient Cas amino acids growth media.

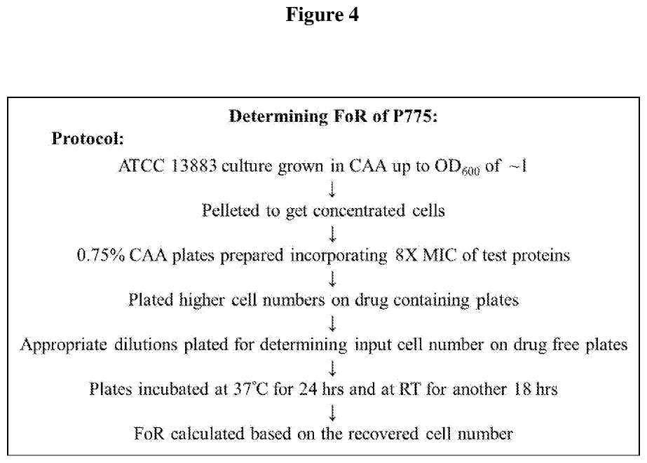

Since P775 harbours the TD and RD of two different klebicins and yet retains activity, P775 may demonstrate properties of both proteins. This was determined by FoR studies as described in FIG. 4.

While the FoR of P628 and P764 were in the expected range of about 10⁻⁷, the FoR of P775 was much lower at 3×10⁻¹⁰ (Table 4).

TABLE 4
FoR of natural klebicins P628, P764 and chimeric klebicin P775

| Proteins | Recovery Frequency |
|---|---|
| P628 | ~6.4 × 10⁻⁷ |
| P764 | ~1 × 10⁻⁷ |
| P775 | 3.1 × 10⁻¹⁰ |

These results indicate: (1) FoR of P775 is ˜10⁻¹⁰, which is significantly lesser than individual klebicins P628 and P764. This could be attributed to the combination of properties of both P628 and P764. (2) P775 behaves like a combination of two klebicins in reducing the FoR.

### Activity of P775 on P628 Resistant Mutants of ATCC13883

Since P775 demonstrated properties of both P628 and P764 by FoR studies, the activity of P775 was determined on P628 resistant mutants.

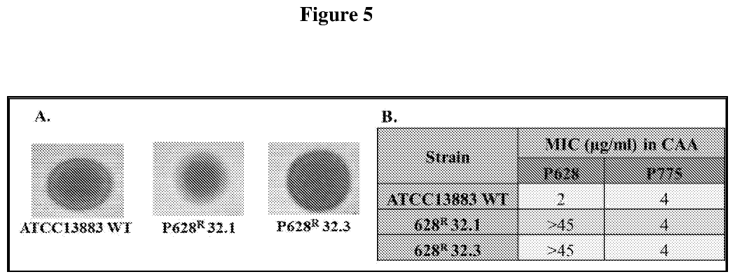

Spontaneous mutants of ATCC13883 that are resistant to klebicin P628 were isolated previously by plating high cell numbers of this strain on P628 embedded agar plates. These mutants were confirmed as P628 resistant by MIC studies. Two of the P628-resistant mutants of ATCC13883 strain, P628R 32.1 and P628R 32.3 were selected for testing activity of P775 along with their wild-type (WT) strain. Lawn inhibition assay and MIC were done for this. Lawn inhibition was done by placing 10 μg of P775 (FIG. 5A) and an MIC was done according to the CLSI protocol with test growth media casamino acids (CAA) (FIG. 5B).

These results indicate that P775 possesses antibacterial activity on P628-resistant mutants of ATCC13883. Taken together, the FoR studies and activity on P628-resistant mutants, P775 exhibits properties of two klebicins.

### Example 2: P810 (P764 TD RD-P774 RD-P764 KD)

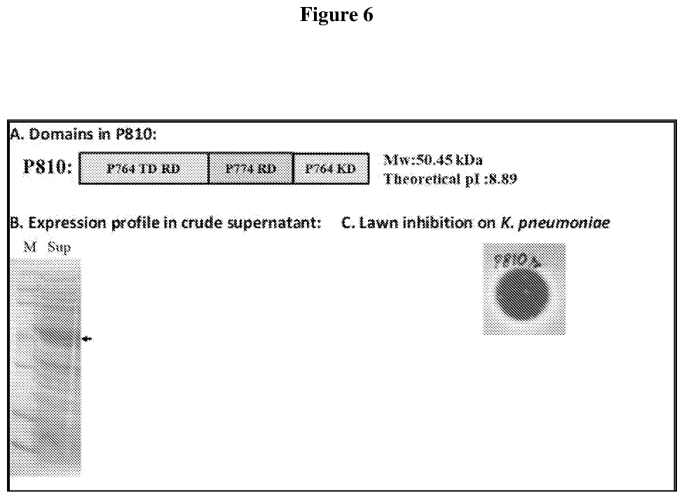

Another strategy is to engineer klebicins to bind to multiple cell surface receptors that can eventually aid in improved coverage as well as reduce FoR. A klebicin with two receptor-binding domains, which bind to two different cell surface receptors, was designed with P764 as the backbone molecule incorporating the receptor binding domain of P774. For this, DNA sequence of the receptor binding domain from klebicin P774 was PCR amplified and cloned into P764, downstream of its own receptor binding region. The fusion protein, termed P810, was expressed in E. coli protein expression strain ER2566 (FIG. 6) and the protein of ˜50 kDa was expressed in the soluble fraction of the cell and was also found be active on K. pneumoniae strain ATCC13883 by a lawn inhibition assay.

These results indicate that P810 is active on K. pneumoniae.

### Example 3: P821 (P628 TD RD-Rigid Linker+P801 FL)

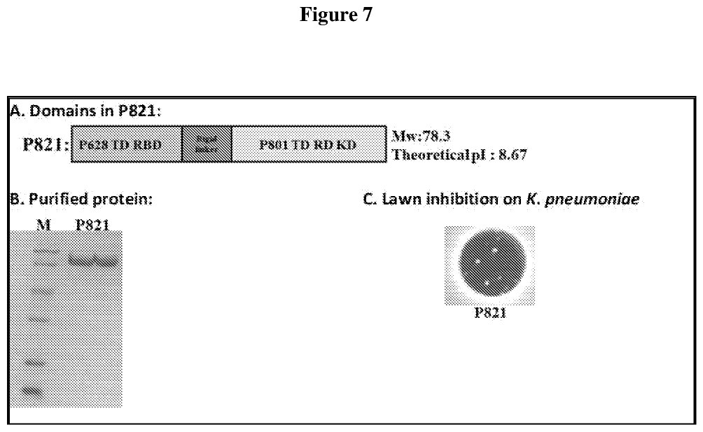

P821 was generated by fusing the TD and RD region of P628 with full length (FL) P801 with the aid of a rigid linker. This construct will have two translocation and receptor binding units that can recognize two different cell surface receptors and translocation machinery. P628 TD RD region and P801 were PCR amplified separately and fused together by synthesis by overlap extension (SOE) PCR. The fusion protein, termed P810, was expressed in E. coli protein expression strain ER2566 (FIG. 7) and the protein of ˜78 kDa was expressed in the soluble fraction of the cell and was also found be active on K. pneumoniae strain ATCC13883 by a lawn inhibition assay.

These results indicate that P821 is active on K. pneumoniae.

### MIC and Bactericidal Activity of P821

The MIC of P821 was determined according to a modified CLSI protocol in growth media, casamino acid media (CAA) and fetal calf serum (FCS). In CAA the MIC was 6 μg/mL and in FCS it was 12 μg/mL, demonstrating the activity of P821 in both growth media and in the presence of serum.

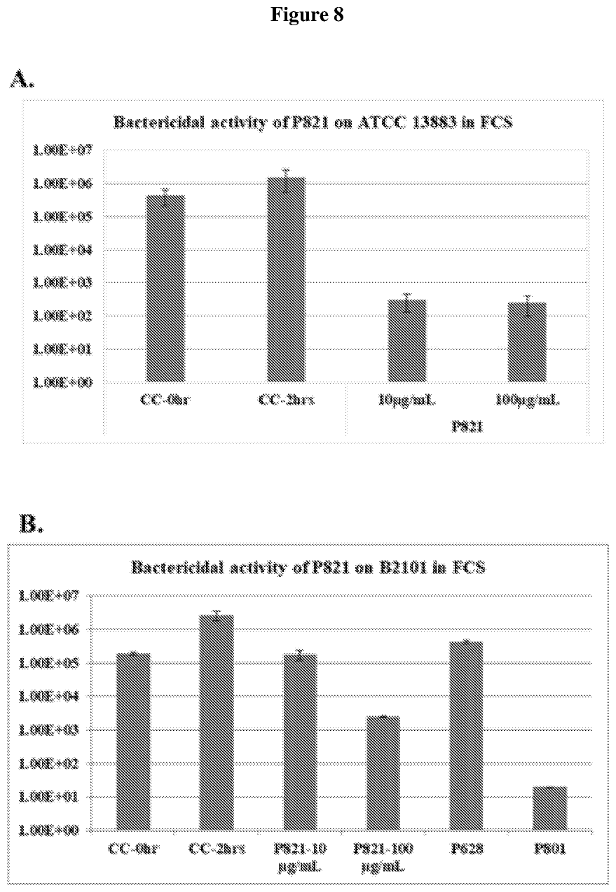

Since P821 is a chimeric fusion of two different klebicins, P628 and P801, to determine whether the fusion has properties of both proteins, the bactericidal activity of P821 was determined on different strains of K. pneumoniae with different sensitivity patterns. ATCC13883 and B2101 were chosen for the first set of experiments. While the strain ATCC13883 is sensitive to both P628 and P801, the strain B2101 is sensitive to only P801. Bactericidal activity was determined by a cell killing assay where in ˜10⁶ CFU/mL of both ATCC13883 and B2101 were treated with 10 and 100 μg/mL of P821 in a volume of 200 μL at 37° C. for 2 h and the remaining number of cells were determined by plating out appropriate dilutions and incubating the plates at 37° C. for 18 h. The bactericidal activity of P628 and P801 on B2101 was determined in the same experiment. 100 μg/mL of both P628 and P801 were used. The results indicate that P821 brought about >3 log reduction in CFU with ATCC13883 even with 10 μg/mL of the purified protein (FIG. 8A). With B2101, ˜2 log CFU reduction was obtained with 100 μg/mL of P821. While 100 μg/mL of P628 did not kill B2101, >3 log CFU reduction was obtained with 100 μg/mL of P801 (FIG. 8B). P821 killed K. pneumoniae strains and the activity was similar to P801.

When a similar experiment was conducted with P628 sensitive-P801 insensitive isolate-B2265, no killing was observed suggesting that the protein did not exhibit P628 activity.

### Example 4: P823 (P628 TD RD-Flexible Linker+P801 FL)

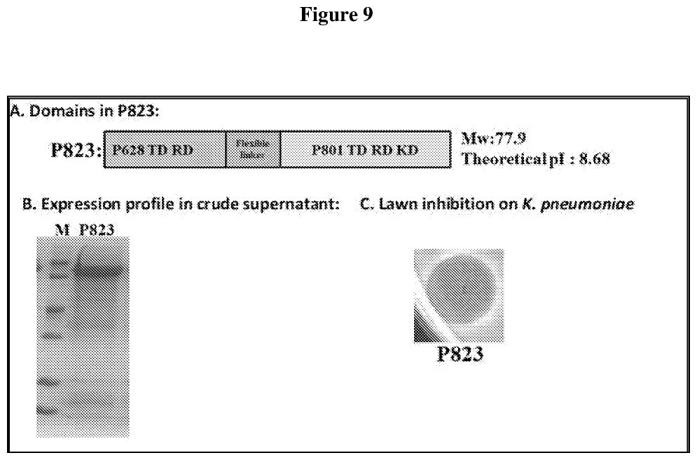

P823 was generated by fusing the TD and RD region of P628 with full length (FL) P801 with the aid of a flexible linker. This construct will have two translocation and receptor binding units that can recognize two different cell surface receptors and translocation machinery. P628 TD RD region and P801 were PCR amplified separately and fused together by synthesis by overlap extension (SOE) PCR. This fusion protein was expressed in E. coli protein expression strain ER2566 (FIG. 9) and the protein of ˜78 kDa was expressed in the soluble fraction of the cell and was also found be active on K. pneumoniae strain ATCC13883 by a lawn inhibition assay.

These results indicate that P823 is active on K. pneumoniae.

### Example 5: P835 (P628 TD RD+P801 FL); P836 (P628 TD RD-4× Rigid Linker+P801 FL); P837 (P628 TD RD-2× Flexible Linker+P801 FL)

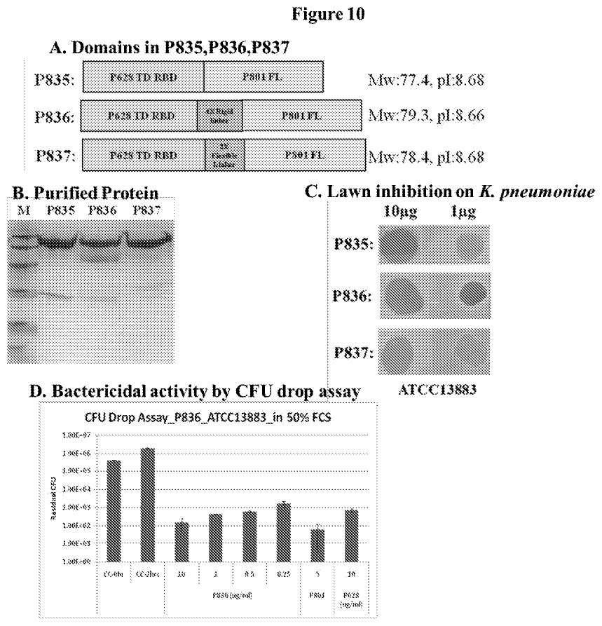

P835, P836, and P837 were generated by fusing the P628TDRD to the N-terminus of Full length P801 without a linker, with a 4× rigid linker, and with a 2× Flexible linker in between, respectively. These constructs have two translocation and receptor binding units that can recognize two different cell surface receptors and translocation machinery. P628 TD RD region and P801 were PCR amplified separately using appropriate primers with the linkers and fused together by synthesis by overlap extension (SOE) PCR (FIG. 10A). The proteins were expressed in E. coli and purified using ion exchange chromatography (FIG. 10B). Purified proteins were then taken forward for testing.

10 μg and 1 μg of the purified proteins were placed on lawns of Klebsiella isolates. The plates were incubated at 30° C. for 16-18 hrs. The fusion proteins showed inhibition zone on the bacterial lawn indicating the fusion was active (FIG. 10C). P836 was active in physiologically relevant condition like serum and exhibited a rapid killing even with 0.25 μg/ml in a 2 hrs CFU drop assay (FIG. 10D).

### Example 6: P845 (P628 FL-Flexible Linker+P801 FL); P862 (P628 FL-Rigid Linker+P801 FL)

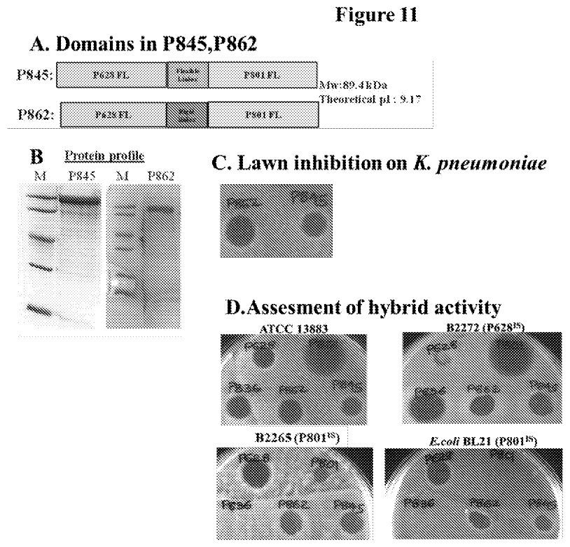

P845 and P862 were generated by fusing the P628 full length to the N-terminus of Full length P801 with a flexible and rigid linker in between respectively (FIG. 11A). P845 and P862 were expressed in E. coli and purified using ion exchange chromatography (FIG. 11B). Purified proteins were then taken forward for testing and compared against P836 to check if there is any improvement in the activity or coverage by extending P628 to full length in P836.

P845 and P862 were tested by lawn inhibition assay. 20 μg of the purified P845 and P862 were placed on lawns of Klebsiella isolates. The plates were incubated at 30° C. for 16-18 hrs. P845 and P862 showed inhibition zone on the bacterial lawn indicating the fusions were active (FIG. 11C).

MICs of P836, P845 and P862 were tested in CAA and 10% FCS against ATCC13883, P845 and P862 gave improved MIC in CAA as compared to P836 and is active even in the presence of serum (Table 5).

TABLE 5
MICs of P836, P845 and P862 in CAA
and 10% FCS on strain ATCC13883

| Protein | CAA(μg/ml) | CAA + 10% FCS(μg/ml) |
|---|---|---|
| P836 | >256 | 4-8 |
| P845 | 4-8 | 4-8 |
| P862 | 4-8 | 4-8 |

The hybrid properties of chimeras were assessed by testing the lawn inhibition activity on P801 insensitive-P628 sensitive isolates and P801 sensitive-P628 insensitive isolates (S-sensitive and IS-insensitive). The isolates chosen were ATCC13883 (Reference standard), B2272 (P801^{S}-P628 ^{IS}), B2265 (P801^{IS}-P628^{S}). Additionally, E. coli was also included for testing since only P628 is active on it. P836 behaved like P801, exhibiting activity only on P801 sensitive isolates and not on P801^{IS}-P628^{S}. However, P845 and P862 were active on both P801^{S}-P628^{IS} and P628 P801^{IS}-P628^{S} isolates and on E. coli also, indicating that these chimeras harbor properties of both P628 and P801 in a single molecule resulting in improved coverage over the individual klebicins alone (FIG. 11D).

### Example 7: P863 (P764 FL-Rigid Linker+P801 FL)

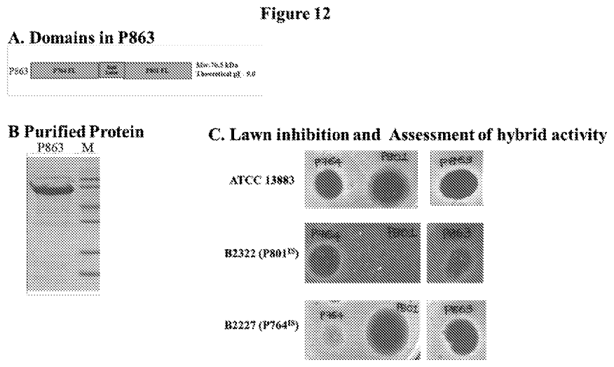

P863 was generated by fusing the P764 full length to the Full length of P801 with a 2× rigid linker in between (FIG. 12A). P863 was expressed in E. coli and purified using ion exchange chromatography (FIG. 12B). Purified protein was then taken forward for testing. 20 μg of the purified P863 was placed on lawns of Klebsiella isolates and the plates were incubated at 30° C. for 16-18 hrs. P863 was active by lawn inhibition assay on strains that were sensitive to P764 but insensitive to P801 and also on strains that were insensitive to P764 and sensitive to P801, indicating that P863 possesses hybrid properties of both P764 and P801 in a single molecule (FIG. 12C). P863 was active even in the presence of serum and gave MIC of 4 μg/ml in CAA and 1 μg/ml with 10% FCS (Fetal Calf Serum) on ATCC 13883.

### Example 8: P867 (Engineered to Improve Potency)

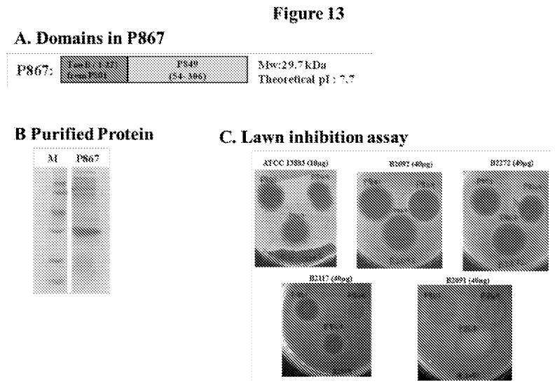

P867 was Generated by Replacing the First 53 Amino Acids of P849 (P801 Homolog that gave no MIC in CAA) with the first 1-22 amino acids (consisting of TonB motif) of P801 (FIG. 13A). P867 was expressed in E. coli and purified using ion exchange chromatography (FIG. 13B). Purified protein was then taken forward for testing. P867 was placed on lawns of Klebsiella isolates and the plates were incubated at 30° C. for 16-18 hrs. P867 was active on the tested P801 sensitive isolates (FIG. 13C). P867 was found to be potent in MIC studies suggesting that the engineering of P849 by introduction of P801 TonB motif has enhanced the activity (Table 6). Activity of P867 was retained in different serum matrices with MIC of 0.5 and 0.07 μg/ml in 50% FCS and 50% human serum respectively on K. pneumoniae strain ATCC13883.

TABLE 6
MIC (μg/mL) in CAA on K. pneumoniae

| Proteins | ATCC13883 | B2092 |
|---|---|---|
| P801 | 1 | 1 |
| P849 | >256 | >256 |
| P867 | 1 | 0.25 |

### Example 9: P870 (P764FL-4× Rigid Linker+P801FL)

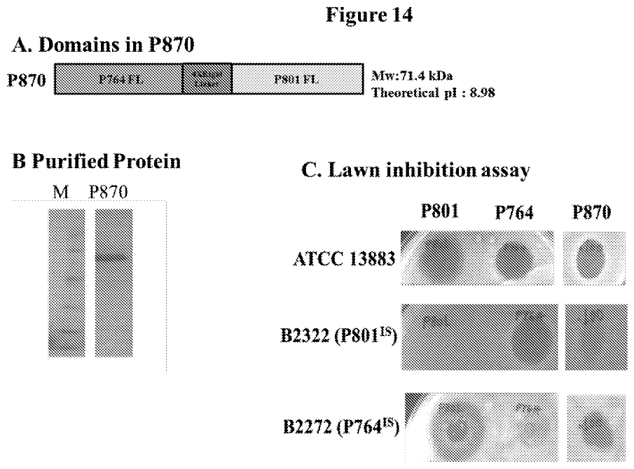

P870 was generated by fusing the P764 full length to the N-terminus of Full length of P801 with a 4× rigid linker in between (FIG. 14A). P870 was expressed in E. coli and was purified using ion exchange chromatography to greater than 90% homogeneity (FIG. 14B). Purified protein was then taken forward for testing. 20 μg of purified P870 was placed on lawns of Klebsiella pneumoniae isolates and the plates were incubated at 30° C. for 16-18 hrs. P870 was active by lawn inhibition assay and the spot clearance was observed on both P764^{IS} and P801^{IS} suggesting hybrid activity of protein (FIG. 14C). The activity was retained in the presence of sera (Table 7).

TABLE 7
MICs of P870
MIC (μg/mL) of P870

| Strains | CAA | 10% FCS |
|---|---|---|
| ATCC13883 | >128 | 1 |
| B2221 | 8 | 16 |
| B2281 | 32 | 32 |

### Example 10: P875 (P801FL-Rigid Linker+GP36 CD)

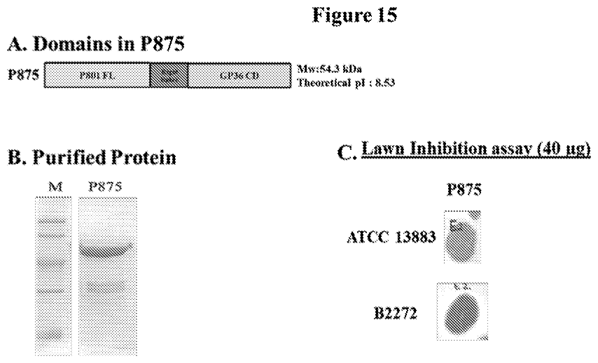

P875 was generated by fusing the GP36 CD (P200), the catalytic domain of the structural lysozyme from P. aeruginosa phage P134 to the C-terminus of Full length P801 (FIG. 15A). GP36 CD has muralytic activity and degrades Gram-negative peptidoglycan only when the cells are devoid of outer membrane. P875 was expressed in E. coli and was purified using ion exchange chromatography (FIG. 15B). The antimicrobial activity of P875 was evaluated by placing 40 μg of the purified P875 on lawns of Klebsiella pneumoniae strains and incubating at 30° C. for 16-18 hrs. P875 showed inhibition zone on the bacterial lawn indicating the fusion was active (FIG. 15C). P875 was highly potent in the presence of 10% FCS and was active on a number of clinical isolates of Klebsiella pneumoniae (KP) (Table 8).

TABLE 8
MIC of P875 on ATCC 13883 and clinical isolates

| Sl. No. | KP isolates | MIC (μg/mL) in 10% FCS |
|---|---|---|
| 1 | ATCC13883 | 0.125 |
| 2 | B2091 | >256 |
| 3 | B2101 | 4 |
| 4 | B2104 | 0.5 |
| 5 | B2113 | >256 |
| 6 | B2117 | >256 |
| 7 | B2123 | 8 |
| 8 | B2151 | 32 |
| 9 | B2253 | 4 |
| 10 | B2556 | 2/4 |
| 11 | B2560 | 8 |
| 12 | B2161 | >256 |
| 13 | B2170 | 0.25 |
| 14 | B2208 | 4/8 |
| 15 | B2261 | 8 |
| 16 | NDM1 KL6 | >256 |
| 17 | B2187 | 4 |
| 18 | B2221 | 4/8 |
| 19 | B2245 | 16 |
| 20 | B2267 | 0.5/1 |
| 21 | B2281 | 16 |

### Example 11: P889 (P764 FL+GP36 CD); P891 (P764FL-Flexible Linker+GP36 CD); P892 (P764-Rigid Linker+GP36 CD)

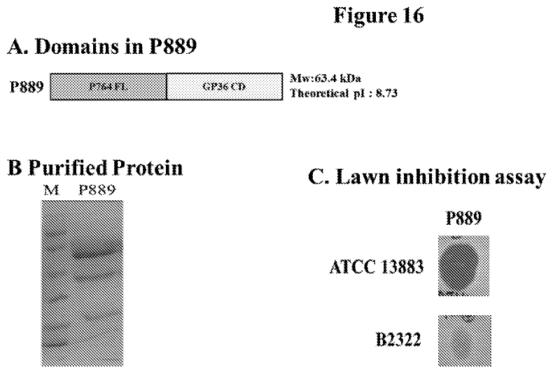

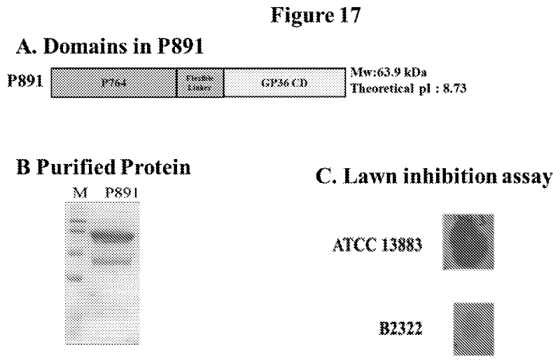

P889 was generated by fusing the GP36 CD (P200), the catalytic domain of the structural lysozyme from P. aeruginosa phage P134 to the C-terminus of Full length P764. P891 and P892 were generated by fusing the GP36 CD (P200) the catalytic domain of the structural lysozyme from P. aeruginosa phage P134 to the C-terminus of Full length P764 with a Flexible linker and a Rigid in between respectively (FIGS. 16A, 17A & 18A). GP36 CD has muralytic activity and degrades Gram-negative peptidoglycan only when the cells are devoid of outer membrane. P889, P891, and P892 were expressed in E. coli and were purified by ion exchange chromatography (FIGS. 16B,17B & 18B). Antimicrobial activity were evaluated by placing 10 μg of purified proteins on the lawns of Klebsiella pneumoniae strains and incubating at 30° C. for 16-18 hrs. P889, P891, and P892 showed inhibition zones on the bacterial lawn indicating that the fusion proteins were active (FIG. 16C,17C,18C). P889, P891, and P892 showed potent activity by MIC assay on clinical isolates of Klebsiella pneumoniae in the presence of FCS (Table 9).

TABLE 9
MIC of P889, P891, and P892 in 10% FCS
against ATCC13883 and clinical isolates

| | P889 MIC | P891 MIC | |
|---|---|---|---|
| Strains | (μg/mL) | (μg/mL) | P892 MIC(μg/mL) |
| ATCC | <0.06 | <0.06 | <0.015 |
| 13883 | | | |
| B2139 | 1 | 0.5 | 2 |
| B2167 | <0.06 | <0.008 | 0.03 |
| B2236 | 0.25 | 0.015 | 0.06 |

All patents, patent applications, and other publications, including GenBank Accession Numbers, cited in this application are incorporated by reference in the entirety for all purposes.

Informal Sequence Listing

```
1. P771 (P764 TD RD-P628 Full length):
Amino acid sequence of P771 (SEQ ID NO: 1):
MPEETLTVVGGGNNSCNVSWGGGNGNNGGAGYSGKYGGTSYEGATSMLKLNDRVLIQLYLCN
PLNPDYIGAPWGSDKDAESIIRANRDKAGKFKANIQNWKTSGTGSLGSPVVGKSYSSGDVDT
YSVSFGKEKYNVLYNRKKDSFTTAYVDGGANKPEHSSGGDGRGPGNSGLGHNGGQASGNVNG
TSGKGGPSSGGGTDPNSGPGWGTTHTPNGDIHNYNPGEFGNGGSKPGGNGGNSGNHSGSSGG
GQSSATAMAFGLPALATPGAEGLALSVSGDALSAAVADVLAALKGPFKFGLWGIAIYGVLPS
EIAKDDPKMMSKIVTSLPADTVTETPVSSLPLDQATVSVTKRVADIVKDERQHIAVVTGRPM
SVPVVDAKPTKRPGVFSVSIPGLPSLQVSVPKGVPAAKAPPKGIIAEKGDSRPAGFTAGGNS
REAVIRFPKESGQKPVYVSVTDVLTPAQVKQRLEEEKRRQQAWDAAHPEEGLKREYDKAKAE
LDAEDKNIATLNSRIASTEKAIPGARAAVQEADKKVKEAEANKDDFVTYNPPHEYGSGWQDQ
VRYLDKDIQNQNEKLKAAQTSLNEMNESLSRDKAALSGAMESRKQKEKKAKDAENKLNEEKK
KPRKGTKDYGHDYFPDPKTEDIKGLGELKEGKPKTPKQGGGGKRARWYGDKKRKIYEWDSQH
GELEGYRASDGEHLGAFDPKTGKQVKGPDPKRNIKKYL
EVSMGLKLNLTWFDKKTEEFKGGEYSKDFGDDGSVIESLGMPLKDNINNGWFDVEKSWVSIL
QPHFKNVIDISKEDYFVSFDYRDGNW
P771 domain boundaries:
      1-38: 764 TD
  39-160: P764 RD
161-479: P628 TD
480-619: P628 RD
620-720: P628 KD
721-808: P628 Immunity
Number of amino acids: 720
Molecular weight: 75940
Theoretical pI: 9.11
DNA Sequence of P771 (SEQ ID NO: 2):
ATGCCTGAAGAAACATTGACTGTCGTAGGTGGTGGTAATAACAGCTGTAATGTTAGCTGGGG
TGGTGGCAATGGTAACAACGGTGGTGCTGGCTATTCTGGTAAATACGGTGGTACTAGCTATG
AAGGTGCAACTAGTATGTTGAAGTTGAATGACCGTGTTTTAATTCAGCTTTATCTTTGCAAT
CCGCTTAACCCAGATTATATTGGAGCACCTTGGGGCAGTGATAAAGATGCAGAATCAATTAT
CAGAGCTAACAGAGATAAAGCAGGAAAGTTCAAGGCTAATATTCAGAACTGGAAAACAAGCG
GTACAGGTTCTTTAGGTAGCCCTGTAGTTGGGAAATCTTATAGCTCAGGTGATGTAGATACT
TATTCGGTTAGCTTCGGAAAAGAAAAATACAATGTCCTGTATAACCGTAAAAAAGACTCTTT
TACTACAGCTTATGTCGATGGCGGCGCAAATAAACCTGAGCATAGTGGTGGAGACGGACGAG
GTCCGGGTAATTCAGGTCTGGGACACAATGGTGGTCAGGCCAGTGGGAATGTGAACGGTACA
TCAGGTAAAGGTGGCCCTTCATCAGGTGGTGGTACGGATCCAAACAGCGGGCCGGGCTGGGG
TACGACGCATACACCTAACGGGGATATTCATAACTATAACCCGGGGGAGTTTGGTAACGGCG
GGAGTAAACCCGGTGGTAATGGCGGTAACAGCGGCAATCATAGCGGGAGCTCTGGTGGCGGA
CAGTCTTCGGCCACCGCGATGGCCTTCGGTCTGCCTGCCTTGGCCACTCCGGGGGCTGAAGG
ACTGGCTTTATCCGTTTCCGGCGATGCGTTGTCGGCCGCTGTTGCTGATGTGCTGGCTGCCC
TGAAAGGTCCGTTTAAGTTTGGTCTGTGGGGGATTGCGATTTACGGAGTGCTGCCTTCTGAG
ATAGCAAAAGATGATCCGAAAATGATGTCAAAAATCGTGACGTCATTGCCGGCAGACACGGT
AACGGAGACGCCGGTCAGCTCCCTGCCGCTGGACCAGGCGACGGTCAGCGTCACTAAACGTG
TGGCGGATATTGTGAAGGACGAGCGGCAGCATATTGCGGTTGTCACCGGCCGGCCAATGAGT
GTTCCTGTGGTGGATGCGAAACCGACAAAACGTCCGGGGGTATTCAGTGTGTCGATTCCGGG
TCTCCCGTCTCTGCAGGTGAGCGTACCTAAAGGTGTTCCGGCCGCGAAAGCCCCGCCAAAAG
GCATTATTGCTGAAAAAGGTGATTCACGTCCGGCTGGTTTTACGGCCGGTGGTAACTCCCGT
GAAGCCGTTATTCGTTTCCCGAAAGAGAGCGGACAGAAACCGGTCTATGTGTCGGTGACGGA
TGTTCTTACTCCGGCACAGGTAAAACAGCGTCTGGAGGAAGAAAAGCGTCGCCAGCAGGCAT
GGGACGCTGCTCACCCGGAAGAGGGGCTGAAAAGAGAGTATGATAAAGCGAAAGCTGAGCTG
GATGCTGAAGATAAAAATATTGCGACCTTAAACAGTCGCATTGCATCGACAGAGAAGGCGAT
CCCCGGTGCAAGGGCTGCTGTTCAGGAAGCCGATAAAAAGGTAAAAGAGGCAGAGGCAAATA
AAGATGATTTTGTGACTTATAACCCGCCTCATGAATATGGCTCCGGGTGGCAGGATCAGGTT
CGCTATCTTGATAAGGATATTCAGAATCAGAATGAGAAATTAAAAGCTGCCCAGACATCTTT
AAACGAAATGAATGAATCCTTATCCAGGGATAAGGCTGCGCTTTCCGGGGCGATGGAGAGCC
GGAAACAAAAGGAGAAAAAAGCGAAGGATGCAGAAAATAAATTAAATGAGGAAAAGAAAAAA
CCTCGTAAGGGAACTAAAGATTACGGCCATGATTATTTTCCTGATCCCAAGACTGAAGATAT
TAAGGGGTTGGGAGAGTTGAAAGAGGGTAAACCTAAAACCCCTAAACAAGGTGGTGGTGGTA
AGCGGGCCCGATGGTATGGTGATAAAAAGCGTAAAATTTATGAATGGGATTCCCAGCACGGT
GAGCTTGAAGGGTACCGCGCCAGTGATGGCGAACACCTCGGGGCATTCGATCCAAAAACGGG
TAAGCAGGTTAAAGGGCCGGATCCAAAACGAAACATTAAAAAATATCTTTAA
GAGGTAAGTATGGGACTTAAATTAAATTTAACCTGGTTTGATAAGAAAACCGAAGAGTTTAA
AGGTGGTGAATACTCAAAAGACTTCGGTGATGATGGTTCTGTCATTGAAAGTCTGGGGATGC
CTTTAAAGGATAATATTAATAATGGTTGGTTTGATGTTGAAAAATCATGGGTTTCGATATTA
CAGCCACACTTTAAAAATGTAATCGATATTAGTAAATTTGATTACTTTGTATCATTTGATTA
TCGGGATGGTAACTGGTAA
2. P772 (P764 TD RD- Flexible linker-P628 FL):
Amino acid sequence of P772 (SEQ ID NO: 3):
MPEETLTVVGGGNNSCNVSWGGGNGNNGGAGYSGKYGGTSYEGATSMLKLNDRVLIQLYLCN
PLNPDYIGAPWGSDKDAESIIRANRDKAGKFKANIQNWKTSGTGSLGSPVVGKSYSSGDVDT
YSVSFGKEKYNVLYNRKKDSFTTAYVDGGANKPEHSGSGSASGSGGDGRGPGNSGLGHNGGQ
ASGNVNGTSGKGGPSSGGGTDPNSGPGWGTTHTPNGDIHNYNPGEFGNGGSKPGGNGGNSGN
HSGSSGGGQSSATAMAFGLPALATPGAEGLALSVSGDALSAAVADVLAALKGPFKFGLWGIA
IYGVLPSEIAKDDPKMMSKIVTSLPADTVTETPVSSLPLDQATVSVTKRVADIVKDERQHIA
VVTGRPMSVPVVDAKPTKRPGVFSVSIPGLPSLQVSVPKGVPAAKAPPKGIIAEKGDSRPAG
FTAGGNSREAVIRFPKESGQKPVYVSVTDVLTPAQVKQRLEEEKRRQQAWDAAHPEEGLKRE
YDKAKAELDAEDKNIATLNSRIASTEKAIPGARAAVQEADKKVKEAEANKDDFVTYNPPHEY
GSGWQDQVRYLDKDIQNQNEKLKAAQTSLNEMNESLSRDKAALSGAMESRKQKEKKAKDAEN
KLNEEKKKPRKGTKDYGHDYFPDPKTEDIKGLGELKEGKPKTPKQGGGGKRARWYGDKKRKI
YEWDSQHGELEGYRASDGEHLGAFDPKTGKQVKGPDPKRNIKKYL
EVSMGLKLNLTWFDKKTEEFKGGEYSKDFGDDGSVIESLGMPLKDNINNGWFDVEKSWVSIL
QPHFKNVIDISKEDYFVSFDYRDGNW
P772 domain boundaries:
      1-38: 764 TD
  39-160: P764 RD
161-167: Flexible linker
168-486: P628 TD
487-626: P628 RD
627-727: P628 KD
727-815: P628 Immunity
Number of amino acids: 727
Molecular weight: 76443
Theoretical pI: 9.11
DNA Sequence of P772 (SEQ ID NO: 4):
ATGCCTGAAGAAACATTGACTGTCGTAGGTGGTGGTAATAACAGCTGTAATGTTAGCTGGGG
TGGTGGCAATGGTAACAACGGTGGTGCTGGCTATTCTGGTAAATACGGTGGTACTAGCTATG
AAGGTGCAACTAGTATGTTGAAGTTGAATGACCGTGTTTTAATTCAGCTTTATCTTTGCAAT
CCGCTTAACCCAGATTATATTGGAGCACCTTGGGGCAGTGATAAAGATGCAGAATCAATTAT
CAGAGCTAACAGAGATAAAGCAGGAAAGTTCAAGGCTAATATTCAGAACTGGAAAACAAGCG
GTACAGGTTCTTTAGGTAGCCCTGTAGTTGGGAAATCTTATAGCTCAGGTGATGTAGATACT
TATTCGGTTAGCTTCGGAAAAGAAAAATACAATGTCCTGTATAACCGTAAAAAAGACTCTTT
TACTACAGCTTATGTCGATGGCGGCGCAAATAAACCTGAGCATGGCTCCGGATCGGCTTCTG
GGAGTGGTGGAGACGGACGAGGTCCGGGTAATTCAGGTCTGGGACACAATGGTGGTCAGGCC
AGTGGGAATGTGAACGGTACATCAGGTAAAGGTGGCCCTTCATCAGGTGGTGGTACGGATCC
AAACAGCGGGCCGGGCTGGGGTACGACGCATACACCTAACGGGGATATTCATAACTATAACC
CGGGGGAGTTTGGTAACGGCGGGAGTAAACCCGGTGGTAATGGCGGTAACAGCGGCAATCAT
AGCGGGAGCTCTGGTGGCGGACAGTCTTCGGCCACCGCGATGGCCTTCGGTCTGCCTGCCTT
GGCCACTCCGGGGGCTGAAGGACTGGCTTTATCCGTTTCCGGCGATGCGTTGTCGGCCGCTG
TTGCTGATGTGCTGGCTGCCCTGAAAGGTCCGTTTAAGTTTGGTCTGTGGGGGATTGCGATT
TACGGAGTGCTGCCTTCTGAGATAGCAAAAGATGATCCGAAAATGATGTCAAAAATCGTGAC
GTCATTGCCGGCAGACACGGTAACGGAGACGCCGGTCAGCTCCCTGCCGCTGGACCAGGCGA
CGGTCAGCGTCACTAAACGTGTGGCGGATATTGTGAAGGACGAGCGGCAGCATATTGCGGTT
GTCACCGGCCGGCCAATGAGTGTTCCTGTGGTGGATGCGAAACCGACAAAACGTCCGGGGGT
ATTCAGTGTGTCGATTCCGGGTCTCCCGTCTCTGCAGGTGAGCGTACCTAAAGGTGTTCCGG
CCGCGAAAGCCCCGCCAAAAGGCATTATTGCTGAAAAAGGTGATTCACGTCCGGCTGGTTTT
ACGGCCGGTGGTAACTCCCGTGAAGCCGTTATTCGTTTCCCGAAAGAGAGCGGACAGAAACC
GGTCTATGTGTCGGTGACGGATGTTCTTACTCCGGCACAGGTAAAACAGCGTCTGGAGGAAG
AAAAGCGTCGCCAGCAGGCATGGGACGCTGCTCACCCGGAAGAGGGGCTGAAAAGAGAGTAT
GATAAAGCGAAAGCTGAGCTGGATGCTGAAGATAAAAATATTGCGACCTTAAACAGTCGCAT
TGCATCGACAGAGAAGGCGATCCCCGGTGCAAGGGCTGCTGTTCAGGAAGCCGATAAAAAGG
TAAAAGAGGCAGAGGCAAATAAAGATGATTTTGTGACTTATAACCCGCCTCATGAATATGGC
TCCGGGTGGCAGGATCAGGTTCGCTATCTTGATAAGGATATTCAGAATCAGAATGAGAAATT
AAAAGCTGCCCAGACATCTTTAAACGAAATGAATGAATCCTTATCCAGGGATAAGGCTGCGC
TTTCCGGGGCGATGGAGAGCCGGAAACAAAAGGAGAAAAAAGCGAAGGATGCAGAAAATAAA
TTAAATGAGGAAAAGAAAAAACCTCGTAAGGGAACTAAAGATTACGGCCATGATTATTTTCC
TGATCCCAAGACTGAAGATATTAAGGGGTTGGGAGAGTTGAAAGAGGGTAAACCTAAAACCC
CTAAACAAGGTGGTGGTGGTAAGCGGGCCCGATGGTATGGTGATAAAAAGCGTAAAATTTAT
GAATGGGATTCCCAGCACGGTGAGCTTGAAGGGTACCGCGCCAGTGATGGCGAACACCTCGG
GGCATTCGATCCAAAAACGGGTAAGCAGGTTAAAGGGCCGGATCCAAAACGAAACATTAAAA
AATATCTTTAA
GAGGTAAGTATGGGACTTAAATTAAATTTAACCTGGTTTGATAAGAAAACCGAAGAGTTTAA
AGGTGGTGAATACTCAAAAGACTTCGGTGATGATGGTTCTGTCATTGAAAGTCTGGGGATGC
CTTTAAAGGATAATATTAATAATGGTTGGTTTGATGTTGAAAAATCATGGGTTTCGATATTA
CAGCCACACTTTAAAAATGTAATCGATATTAGTAAATTTGATTACTTTGTATCATTTGATTA
TCGGGATGGTAACTGGTAA
3. P777 (P764 TD RD-Rigid linker-P628 FL):
Amino acid sequence of P777 (SEQ ID NO: 5):
MPEETLTVVGGGNNSCNVSWGGGNGNNGGAGYSGKYGGTSYEGATSMLKLNDRVLIQLYLCN
PLNPDYIGAPWGSDKDAESIIRANRDKAGKFKANIQNWKTSGTGSLGSPVVGKSYSSGDVDT
YSVSFGKEKYNVLYNRKKDSFTTAYVDGGANKPEHSEAAAKEAAAKSGGDGRGPGNSGLGHN
GGQASGNVNGTSGKGGPSSGGGTDPNSGPGWGTTHTPNGDIHNYNPGEFGNGGSKPGGNGGN
SGNHSGSSGGGQSSATAMAFGLPALATPGAEGLALSVSGDALSAAVADVLAALKGPFKFGLW
GIAIYGVLPSEIAKDDPKMMSKIVTSLPADTVTETPVSSLPLDQATVSVTKRVADIVKDERQ
HIAVVTGRPMSVPVVDAKPTKRPGVFSVSIPGLPSLQVSVPKGVPAAKAPPKGIIAEKGDSR
PAGFTAGGNSREAVIRFPKESGQKPVYVSVTDVLTPAQVKQRLEEEKRRQQAWDAAHPEEGL
KREYDKAKAELDAEDKNIATLNSRIASTEKAIPGARAAVQEADKKVKEAEANKDDFVTYNPP
HEYGSGWQDQVRYLDKDIQNQNEKLKAAQTSLNEMNESLSRDKAALSGAMESRKQKEKKAKD
AENKLNEEKKKPRKGTKDYGHDYFPDPKTEDIKGLGELKEGKPKTPKQGGGGKRARWYGDKK
RKIYEWDSQHGELEGYRASDGEHLGAFDPKTGKQVKGPDPKRNIKKYL
EVSMGLKLNLTWFDKKTEEFKGGEYSKDFGDDGSVIESLGMPLKDNINNGWFDVEKSWVSIL
QPHFKNVIDISKFDYFVSFDYRDGNW
P777 domain boundaries:
      1-38: 764 TD
  39-160: P764 RD
161-170: Rigid linker
171-489: P628 TD
490-629: P628 RD
630-730: P628 KD
731-818: P628 Immunity
Number of amino acids: 730
Molecular weight: 76881
Theoretical pI: 9.10
DNA sequence of P777 (SEQ ID NO: 6):
ATGCCTGAAGAAACATTGACTGTCGTAGGTGGTGGTAATAACAGCTGTAATGTTAGCTGGGG
TGGTGGCAATGGTAACAACGGTGGTGCTGGCTATTCTGGTAAATACGGTGGTACTAGCTATG
AAGGTGCAACTAGTATGTTGAAGTTGAATGACCGTGTTTTAATTCAGCTTTATCTTTGCAAT
CCGCTTAACCCAGATTATATTGGAGCACCTTGGGGCAGTGATAAAGATGCAGAATCAATTAT
CAGAGCTAACAGAGATAAAGCAGGAAAGTTCAAGGCTAATATTCAGAACTGGAAAACAAGCG
GTACAGGTTCTTTAGGTAGCCCTGTAGTTGGGAAATCTTATAGCTCAGGTGATGTAGATACT
TATTCGGTTAGCTTCGGAAAAGAAAAATACAATGTCCTGTATAACCGTAAAAAAGACTCTTT
TACTACAGCTTATGTCGATGGCGGCGCAAATAAACCTGAGCATGAAGCCGCTGCCAAGGAGG
CGGCCGCTAAAAGTGGTGGAGACGGACGAGGTCCGGGTAATTCAGGTCTGGGACACAATGGT
GGTCAGGCCAGTGGGAATGTGAACGGTACATCAGGTAAAGGTGGCCCTTCATCAGGTGGTGG
TACGGATCCAAACAGCGGGCCGGGCTGGGGTACGACGCATACACCTAACGGGGATATTCATA
ACTATAACCCGGGGGAGTTTGGTAACGGCGGGAGTAAACCCGGTGGTAATGGCGGTAACAGC
GGCAATCATAGCGGGAGCTCTGGTGGCGGACAGTCTTCGGCCACCGCGATGGCCTTCGGTCT
GCCTGCCTTGGCCACTCCGGGGGCTGAAGGACTGGCTTTATCCGTTTCCGGCGATGCGTTGT
CGGCCGCTGTTGCTGATGTGCTGGCTGCCCTGAAAGGTCCGTTTAAGTTTGGTCTGTGGGGG
ATTGCGATTTACGGAGTGCTGCCTTCTGAGATAGCAAAAGATGATCCGAAAATGATGTCAAA
AATCGTGACGTCATTGCCGGCAGACACGGTAACGGAGACGCCGGTCAGCTCCCTGCCGCTGG
ACCAGGCGACGGTCAGCGTCACTAAACGTGTGGCGGATATTGTGAAGGACGAGCGGCAGCAT
ATTGCGGTTGTCACCGGCCGGCCAATGAGTGTTCCTGTGGTGGATGCGAAACCGACAAAACG
TCCGGGGGTATTCAGTGTGTCGATTCCGGGTCTCCCGTCTCTGCAGGTGAGCGTACCTAAAG
GTGTTCCGGCCGCGAAAGCCCCGCCAAAAGGCATTATTGCTGAAAAAGGTGATTCACGTCCG
GCTGGTTTTACGGCCGGTGGTAACTCCCGTGAAGCCGTTATTCGTTTCCCGAAAGAGAGCGG
ACAGAAACCGGTCTATGTGTCGGTGACGGATGTTCTTACTCCGGCACAGGTAAAACAGCGTC
TGGAGGAAGAAAAGCGTCGCCAGCAGGCATGGGACGCTGCTCACCCGGAAGAGGGGCTGAAA
AGAGAGTATGATAAAGCGAAAGCTGAGCTGGATGCTGAAGATAAAAATATTGCGACCTTAAA
CAGTCGCATTGCATCGACAGAGAAGGCGATCCCCGGTGCAAGGGCTGCTGTTCAGGAAGCCG
ATAAAAAGGTAAAAGAGGCAGAGGCAAATAAAGATGATTTTGTGACTTATAACCCGCCTCAT
GAATATGGCTCCGGGTGGCAGGATCAGGTTCGCTATCTTGATAAGGATATTCAGAATCAGAA
TGAGAAATTAAAAGCTGCCCAGACATCTTTAAACGAAATGAATGAATCCTTATCCAGGGATA
AGGCTGCGCTTTCCGGGGCGATGGAGAGCCGGAAACAAAAGGAGAAAAAAGCGAAGGATGCA
GAAAATAAATTAAATGAGGAAAAGAAAAAACCTCGTAAGGGAACTAAAGATTACGGCCATGA
TTATTTTCCTGATCCCAAGACTGAAGATATTAAGGGGTTGGGAGAGTTGAAAGAGGGTAAAC
CTAAAACCCCTAAACAAGGTGGTGGTGGTAAGCGGGCCCGATGGTATGGTGATAAAAAGCGT
AAAATTTATGAATGGGATTCCCAGCACGGTGAGCTTGAAGGGTACCGCGCCAGTGATGGCGA
ACACCTCGGGGCATTCGATCCAAAAACGGGTAAGCAGGTTAAAGGGCCGGATCCAAAACGAA
ACATTAAAAAATATCTTTAA
GAGGTAAGTATGGGACTTAAATTAAATTTAACCTGGTTTGATAAGAAAACCGAAGAGTTTAA
AGGTGGTGAATACTCAAAAGACTTCGGTGATGATGGTTCTGTCATTGAAAGTCTGGGGATGC
CTTTAAAGGATAATATTAATAATGGTTGGTTTGATGTTGAAAAATCATGGGTTTCGATATTA
CAGCCACACTTTAAAAATGTAATCGATATTAGTAAATTTGATTACTTTGTATCATTTGATTA
TCGGGATGGTAACTGGTAA
4. P775 (P628 TD and RD fusion to P764 full-length with rigid linker):
Amino acid sequence of P775 (SEQ ID NO: 7):
MSGGDGRGPGNSGLGHNGGQASGNVNGTSGKGGPSSGGGTDPNSGPGWGTTHTPNGDIHNYN
PGEFGNGGSKPGGNGGNSGNHSGSSGGGQSSATAMAFGLPALATPGAEGLALSVSGDALSAA
VADVLAALKGPFKFGLWGIAIYGVLPSEIAKDDPKMMSKIVTSLPADTVTETPVSSLPLDQA
TVSVTKRVADIVKDERQHIAVVTGRPMSVPVVDAKPTKRPGVFSVSIPGLPSLQVSVPKGVP
AAKAPPKGIIAEKGDSRPAGFTAGGNSREAVIRFPKESGQKPVYVSVTDVLTPAQVKQRLEE
EKRRQQAWDAAHPEEGLKREYDKAKAELDAEDKNIATLNSRIASTEKAIPGARAAVQEADKK
VKEAEANKDDFVTYNPPHEYGSGWQDQVRYLDKDIQNQNEKLKAAQTSLNEMNESLSRDKAA
LSGAMESRKQKEKKAKDAENKLNEEKEAAAKEAAAKPEETLTVVGGGNNSCNVSWGGGNGNN
GGAGYSGKYGGTSYEGATSMLKLNDRVLIQLYLCNPLNPDYIGAPWGSDKDAESIIRANRDK
AGKFKANIQNWKTSGTGSLGSPVVGKSYSSGDVDTYSVSFGKEKYNVLYNRKKDSFTTAYVD
GGANKPEHSMKDQAIAVVKLYLLNESQASVIDTTSGIITDSGKTLSGKLGDKYNTLAKEAAD
NIKNFQGKKLRSFNDAMASINELANNPKMKLSQADKTVVSNALKQMDLSALADRFKGLEKAF
TWGDRLLKAEKIREGVVTGITTGDWQKLAFEVEAMYLSGVAGAVALGITTAMISTVAVALSL
PAVAVSALTVVSVIGISILTSYIDADKAKALNNAVLGLFK
P775 Domain boundaries:
    1-320: 628 TD
321-460: 628 RD
461-470: rigid linker
471-507: 764 TD
508-629: 764 RD
630-846: 764 KD
Number of amino acids: 846
Molecular weight: 88454
Theoretical pI: 8.77
DNA sequence of P775 (SEQ ID NO: 8):
ATGAGTGGTGGAGACGGACGAGGTCCGGGTAATTCAGGTCTGGGACACAATGGT
GGTCAGGCCAGTGGGAATGTGAACGGTACATCAGGTAAAGGTGGCCCTTCATCA
GGTGGTGGTACGGATCCAAACAGCGGGCCGGGCTGGGGTACGACGCATACACCT
AACGGGGATATTCATAACTATAACCCGGGGGAGTTTGGTAACGGCGGGAGTAAA
CCCGGTGGTAATGGCGGTAACAGCGGCAATCATAGCGGGAGCTCTGGTGGCGGA
CAGTCTTCGGCCACCGCGATGGCCTTCGGTCTGCCTGCCTTGGCCACTCCGGGGG
CTGAAGGACTGGCTTTATCCGTTTCCGGCGATGCGTTGTCGGCCGCTGTTGCTGA
TGTGCTGGCTGCCCTGAAAGGTCCGTTTAAGTTTGGTCTGTGGGGGATTGCGATT
TACGGAGTGCTGCCTTCTGAGATAGCAAAAGATGATCCGAAAATGATGTCAAAA
ATCGTGACGTCATTGCCGGCAGACACGGTAACGGAGACGCCGGTCAGCTCCCTG
CCGCTGGACCAGGCGACGGTCAGCGTCACTAAACGTGTGGCGGATATTGTGAAG
GACGAGCGGCAGCATATTGCGGTTGTCACCGGCCGGCCAATGAGTGTTCCTGTGG
TGGATGCGAAACCGACAAAACGTCCGGGGGTATTCAGTGTGTCGATTCCGGGTCT
CCCGTCTCTGCAGGTGAGCGTACCTAAAGGTGTTCCGGCCGCGAAAGCCCCGCCA
AAAGGCATTATTGCTGAAAAAGGTGATTCACGTCCGGCTGGTTTTACGGCCGGTG
GTAACTCCCGTGAAGCCGTTATTCGTTTCCCGAAAGAGAGCGGACAGAAACCGG
TCTATGTGTCGGTGACGGATGTTCTTACTCCGGCACAGGTAAAACAGCGTCTGGA
GGAAGAAAAGCGTCGCCAGCAGGCATGGGACGCTGCTCACCCGGAAGAGGGGC
TGAAAAGAGAGTATGATAAAGCGAAAGCTGAGCTGGATGCTGAAGATAAAAAT
ATTGCGACCTTAAACAGTCGCATTGCATCGACAGAGAAGGCGATCCCCGGTGCA
AGGGCTGCTGTTCAGGAAGCCGATAAAAAGGTAAAAGAGGCAGAGGCAAATAA
AGATGATTTTGTGACTTATAACCCGCCTCATGAATATGGCTCCGGGTGGCAGGAT
CAGGTTCGCTATCTTGATAAGGATATTCAGAATCAGAATGAGAAATTAAAAGCT
GCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGC
AGAAAATAAATTAAATGAGGAAAAGGAAGCCGCTGCCAAGGAGGCGGCCGCTA
AACCTGAAGAAACATTGACTGTCGTAGGTGGTGGTAATAACAGCTGTAATGTTA
GCTGGGGTGGTGGCAATGGTAACAACGGTGGTGCTGGCTATTCTGGTAAATACG
GTGGTACTAGCTATGAAGGTGCAACTAGTATGTTGAAGTTGAATGACCGTGTTTT
AATTCAGCTTTATCTTTGCAATCCGCTTAACCCAGATTATATTGGAGCACCTTGGG
GCAGTGATAAAGATGCAGAATCAATTATCAGAGCTAACAGAGATAAAGCAGGAA
AGTTCAAGGCTAATATTCAGAACTGGAAAACAAGCGGTACAGGTTCTTTAGGTA
GCCCTGTAGTTGGGAAATCTTATAGCTCAGGTGATGTAGATACTTATTCGGTTAG
CTTCGGAAAAGAAAAATACAATGTCCTGTATAACCGTAAAAAAGACTCTTTTACT
ACAGCTTATGTCGATGGCGGCGCAAATAAACCTGAGCATAGCATGAAGGATCAG
GCTATTGCCGTTGTTAAACTTTACCTTCTCAACGAAAGTCAGGCATCGGTAATCG
ATACTACATCGGGAATTATTACTGACTCTGGTAAAACTCTTAGCGGGAAATTAGG
TGATAAATACAACACTCTGGCGAAAGAAGCTGCTGACAATATAAAAAACTTCCA
GGGTAAGAAACTCCGCAGTTTTAATGATGCTATGGCATCTATTAATGAACTAGCT
AACAATCCAAAGATGAAGTTAAGTCAGGCGGATAAAACAGTCGTTTCTAATGCC
CTCAAACAAATGGATTTGTCAGCACTAGCTGACCGATTCAAAGGGTTAGAGAAA
GCCTTTACTTGGGGTGATCGACTTCTTAAAGCCGAGAAAATCAGAGAAGGTGTTG
TTACTGGTATTACCACAGGGGACTGGCAAAAGCTGGCGTTTGAGGTTGAAGCTAT
GTACCTCAGTGGTGTTGCTGGCGCCGTAGCGTTAGGGATTACTACTGCCATGATT
AGCACAGTCGCAGTCGCTTTGTCACTTCCAGCTGTAGCTGTCTCTGCGCTTACTGT
TGTGTCCGTCATTGGCATCTCTATTCTCACATCTTATATCGATGCTGATAAGGCCA
AAGCACTGAATAATGCAGTGCTTGGCTTATTTAAATAA
5. P776 (P628 TD RD-Flexible linker-P764 FL):
Amino acid sequence of P776 (SEQ ID NO: 9):
MSGGDGRGPGNSGLGHNGGQASGNVNGTSGKGGPSSGGGTDPNSGPGWGTTHTPNGDIHNYN
PGEFGNGGSKPGGNGGNSGNHSGSSGGGQSSATAMAFGLPALATPGAEGLALSVSGDALSAA
VADVLAALKGPFKFGLWGIAIYGVLPSEIAKDDPKMMSKIVTSLPADTVTETPVSSLPLDQA
TVSVTKRVADIVKDERQHIAVVTGRPMSVPVVDAKPTKRPGVFSVSIPGLPSLQVSVPKGVP
AAKAPPKGIIAEKGDSRPAGFTAGGNSREAVIRFPKESGQKPVYVSVTDVLTPAQVKQRLEE
EKRRQQAWDAAHPEEGLKREYDKAKAELDAEDKNIATLNSRIASTEKAIPGARAAVQEADKK
VKEAEANKDDFVTYNPPHEYGSGWQDQVRYLDKDIQNQNEKLKAAQTSLNEMNESLSRDKAA
LSGAMESRKQKEKKAKDAENKLNEEKGSGSASGPEETLTVVGGGNNSCNVSWGGGNGNNGGA
GYSGKYGGTSYEGATSMLKLNDRVLIQLYLCNPLNPDYIGAPWGSDKDAESIIRANRDKAGK
FKANIQNWKTSGTGSLGSPVVGKSYSSGDVDTYSVSFGKEKYNVLYNRKKDSFTTAYVDGGA
NKPEHSMKDQAIAVVKLYLLNESQASVIDTTSGIITDSGKTLSGKLGDKYNTLAKEAADNIK
NFQGKKLRSFNDAMASINELANNPKMKLSQADKTVVSNALKQMDLSALADRFKGLEKAFTWG
DRLLKAEKIREGVVTGITTGDWQKLAFEVEAMYLSGVAGAVALGITTAMISTVAVALSLPAV
AVSALTVVSVIGISILTSYIDADKAKALNNAVLGLFK
P776 Domain boundaries:
    1-320: 628 TD
321-460: 628 RD
461-467: Flexible linker
468-504: 764 TD
505-626: 764 RD
627-843: 764 KD
Number of amino acids: 843
Molecular weight: 87990
Theoretical pI: 8.77
DNA sequence of P776 (SEQ ID NO: 10):
ATGAGTGGTGGAGACGGACGAGGTCCGGGTAATTCAGGTCTGGGACACAATGGTGGTCAGGC
CAGTGGGAATGTGAACGGTACATCAGGTAAAGGTGGCCCTTCATCAGGTGGTGGTACGGATC
CAAACAGCGGGCCGGGCTGGGGTACGACGCATACACCTAACGGGGATATTCATAACTATAAC
CCGGGGGAGTTTGGTAACGGCGGGAGTAAACCCGGTGGTAATGGCGGTAACAGCGGCAATCA
TAGCGGGAGCTCTGGTGGCGGACAGTCTTCGGCCACCGCGATGGCCTTCGGTCTGCCTGCCT
TGGCCACTCCGGGGGCTGAAGGACTGGCTTTATCCGTTTCCGGCGATGCGTTGTCGGCCGCT
GTTGCTGATGTGCTGGCTGCCCTGAAAGGTCCGTTTAAGTTTGGTCTGTGGGGGATTGCGAT
TTACGGAGTGCTGCCTTCTGAGATAGCAAAAGATGATCCGAAAATGATGTCAAAAATCGTGA
CGTCATTGCCGGCAGACACGGTAACGGAGACGCCGGTCAGCTCCCTGCCGCTGGACCAGGCG
ACGGTCAGCGTCACTAAACGTGTGGCGGATATTGTGAAGGACGAGCGGCAGCATATTGCGGT
TGTCACCGGCCGGCCAATGAGTGTTCCTGTGGTGGATGCGAAACCGACAAAACGTCCGGGGG
TATTCAGTGTGTCGATTCCGGGTCTCCCGTCTCTGCAGGTGAGCGTACCTAAAGGTGTTCCG
GCCGCGAAAGCCCCGCCAAAAGGCATTATTGCTGAAAAAGGTGATTCACGTCCGGCTGGTTT
TACGGCCGGTGGTAACTCCCGTGAAGCCGTTATTCGTTTCCCGAAAGAGAGCGGACAGAAAC
CGGTCTATGTGTCGGTGACGGATGTTCTTACTCCGGCACAGGTAAAACAGCGTCTGGAGGAA
GAAAAGCGTCGCCAGCAGGCATGGGACGCTGCTCACCCGGAAGAGGGGCTGAAAAGAGAGTA
TGATAAAGCGAAAGCTGAGCTGGATGCTGAAGATAAAAATATTGCGACCTTAAACAGTCGCA
TTGCATCGACAGAGAAGGCGATCCCCGGTGCAAGGGCTGCTGTTCAGGAAGCCGATAAAAAG
GTAAAAGAGGCAGAGGCAAATAAAGATGATTTTGTGACTTATAACCCGCCTCATGAATATGG
CTCCGGGTGGCAGGATCAGGTTCGCTATCTTGATAAGGATATTCAGAATCAGAATGAGAAAT
TAAAAGCTGCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGCAGAAAATAA
ATTAAATGAGGAAAAGGGCTCCGGATCGGCTTCTGGGCCTGAAGAAACATTGACTGTCGTAG
GTGGTGGTAATAACAGCTGTAATGTTAGCTGGGGTGGTGGCAATGGTAACAACGGTGGTGCT
GGCTATTCTGGTAAATACGGTGGTACTAGCTATGAAGGTGCAACTAGTATGTTGAAGTTGAA
TGACCGTGTTTTAATTCAGCTTTATCTTTGCAATCCGCTTAACCCAGATTATATTGGAGCAC
CTTGGGGCAGTGATAAAGATGCAGAATCAATTATCAGAGCTAACAGAGATAAAGCAGGAAAG
TTCAAGGCTAATATTCAGAACTGGAAAACAAGCGGTACAGGTTCTTTAGGTAGCCCTGTAGT
TGGGAAATCTTATAGCTCAGGTGATGTAGATACTTATTCGGTTAGCTTCGGAAAAGAAAAAT
ACAATGTCCTGTATAACCGTAAAAAAGACTCTTTTACTACAGCTTATGTCGATGGCGGCGCA
AATAAACCTGAGCATAGCATGAAGGATCAGGCTATTGCCGTTGTTAAACTTTACCTTCTCAA
CGAAAGTCAGGCATCGGTAATCGATACTACATCGGGAATTATTACTGACTCTGGTAAAACTC
TTAGCGGGAAATTAGGTGATAAATACAACACTCTGGCGAAAGAAGCTGCTGACAATATAAAA
AACTTCCAGGGTAAGAAACTCCGCAGTTTTAATGATGCTATGGCATCTATTAATGAACTAGC
TAACAATCCAAAGATGAAGTTAAGTCAGGCGGATAAAACAGTCGTTTCTAATGCCCTCAAAC
AAATGGATTTGTCAGCACTAGCTGACCGATTCAAAGGGTTAGAGAAAGCCTTTACTTGGGGT
GATCGACTTCTTAAAGCCGAGAAAATCAGAGAAGGTGTTGTTACTGGTATTACCACAGGGGA
CTGGCAAAAGCTGGCGTTTGAGGTTGAAGCTATGTACCTCAGTGGTGTTGCTGGCGCCGTAG
CGTTAGGGATTACTACTGCCATGATTAGCACAGTCGCAGTCGCTTTGTCACTTCCAGCTGTA
GCTGTCTCTGCGCTTACTGTTGTGTCCGTCATTGGCATCTCTATTCTCACATCTTATATCGA
TGCTGATAAGGCCAAAGCACTGAATAATGCAGTGCTTGGCTTATTTAAATAA
6. P780 (P774 TD RD-Rigid linker-P628 FL):
Amino acid sequence of P780 (SEQ ID NO: 11):
MSETMVVVAPPTGFEPAGYGGGLFSPSTPNNSPSQGQIFLQVTLPYYQSAKFCQDSMAWLAQ
YVKTHGATDPLTIQVVANNIRYFLNADTNLCHNPRQNVWEAFHSEMTGSGPAPAKYDYKSMS
LKQEAAAKEAAAKSGGDGRGPGNSGLGHNGGQASGNVNGTSGKGGPSSGGGTDPNSGPGWGT
THTPNGDIHNYNPGEFGNGGSKPGGNGGNSGNHSGSSGGGQSSATAMAFGLPALATPGAEGL
ALSVSGDALSAAVADVLAALKGPFKFGLWGIAIYGVLPSEIAKDDPKMMSKIVTSLPADTVT
ETPVSSLPLDQATVSVTKRVADIVKDERQHIAVVTGRPMSVPVVDAKPTKRPGVFSVSIPGL
PSLQVSVPKGVPAAKAPPKGIIAEKGDSRPAGFTAGGNSREAVIRFPKESGQKPVYVSVTDV
LTPAQVKQRLEEEKRRQQAWDAAHPEEGLKREYDKAKAELDAEDKNIATLNSRIASTEKAIP
GARAAVQEADKKVKEAEANKDDFVTYNPPHEYGSGWQDQVRYLDKDIQNQNEKLKAAQTSLN
EMNESLSRDKAALSGAMESRKQKEKKAKDAENKLNEEKKKPRKGTKDYGHDYFPDPKTEDIK
GLGELKEGKPKTPKQGGGGKRARWYGDKKRKIYEWDSQHGELEGYRASDGEHLGAFDPKTGK
QVKGPDPKRNIKKYL
EVSMGLKLNLTWFDKKTEEFKGGEYSKDFGDDGSVIESLGMPLKDNINNGWFDVEKSWVSIL
QPHFKNVIDISKFDYFVSFDYRDGNW
P780 boundaries:
      1-34: P774 TD
  35-127: P774 RD
128-137: Rigid linker
138-456: P628 TD
457-596: P628 RD
597-697: P628 KD
698-785: P628 Immunity
Number of amino acids: 697
Molecular weight: 73983
Theoretical pI: 9.01
DNA sequence of P780 (SEQ ID NO: 12):
ATGAGCGAGACCATGGTTGTTGTTGCGCCGCCGACCGGTTTTGAGCCGGCGGGTTACGGTGG
TGGCCTGTTCAGCCCGAGCACCCCGAACAACAGCCCGAGCCAGGGTCAAATCTTCCTGCAAG
TGACCCTGCCGTACTATCAGAGCGCGAAGTTTTGCCAAGACAGCATGGCGTGGCTGGCGCAG
TACGTTAAAACCCACGGCGCGACCGATCCGCTGACCATCCAAGTGGTTGCGAACAACATTCG
TTACTTCCTGAACGCGGACACCAACCTGTGCCACAACCCGCGTCAGAACGTGTGGGAGGCGT
TTCACAGCGAAATGACCGGTAGCGGTCCGGCGCCGGCGAAGTACGATTATAAAAGCATGAGC
CTGAAGCAAGAAGCCGCTGCCAAGGAGGCGGCCGCTAAAAGTGGTGGAGACGGACGAGGTCC
GGGTAATTCAGGTCTGGGACACAATGGTGGTCAGGCCAGTGGGAATGTGAACGGTACATCAG
GTAAAGGTGGCCCTTCATCAGGTGGTGGTACGGATCCAAACAGCGGGCCGGGCTGGGGTACG
ACGCATACACCTAACGGGGATATTCATAACTATAACCCGGGGGAGTTTGGTAACGGCGGGAG
TAAACCCGGTGGTAATGGCGGTAACAGCGGCAATCATAGCGGGAGCTCTGGTGGCGGACAGT
CTTCGGCCACCGCGATGGCCTTCGGTCTGCCTGCCTTGGCCACTCCGGGGGCTGAAGGACTG
GCTTTATCCGTTTCCGGCGATGCGTTGTCGGCCGCTGTTGCTGATGTGCTGGCTGCCCTGAA
AGGTCCGTTTAAGTTTGGTCTGTGGGGGATTGCGATTTACGGAGTGCTGCCTTCTGAGATAG
CAAAAGATGATCCGAAAATGATGTCAAAAATCGTGACGTCATTGCCGGCAGACACGGTAACG
GAGACGCCGGTCAGCTCCCTGCCGCTGGACCAGGCGACGGTCAGCGTCACTAAACGTGTGGC
GGATATTGTGAAGGACGAGCGGCAGCATATTGCGGTTGTCACCGGCCGGCCAATGAGTGTTC
CTGTGGTGGATGCGAAACCGACAAAACGTCCGGGGGTATTCAGTGTGTCGATTCCGGGTCTC
CCGTCTCTGCAGGTGAGCGTACCTAAAGGTGTTCCGGCCGCGAAAGCCCCGCCAAAAGGCAT
TATTGCTGAAAAAGGTGATTCACGTCCGGCTGGTTTTACGGCCGGTGGTAACTCCCGTGAAG
CCGTTATTCGTTTCCCGAAAGAGAGCGGACAGAAACCGGTCTATGTGTCGGTGACGGATGTT
CTTACTCCGGCACAGGTAAAACAGCGTCTGGAGGAAGAAAAGCGTCGCCAGCAGGCATGGGA
CGCTGCTCACCCGGAAGAGGGGCTGAAAAGAGAGTATGATAAAGCGAAAGCTGAGCTGGATG
CTGAAGATAAAAATATTGCGACCTTAAACAGTCGCATTGCATCGACAGAGAAGGCGATCCCC
GGTGCAAGGGCTGCTGTTCAGGAAGCCGATAAAAAGGTAAAAGAGGCAGAGGCAAATAAAGA
TGATTTTGTGACTTATAACCCGCCTCATGAATATGGCTCCGGGTGGCAGGATCAGGTTCGCT
ATCTTGATAAGGATATTCAGAATCAGAATGAGAAATTAAAAGCTGCCCAGACATCTTTAAAC
GAAATGAATGAATCCTTATCCAGGGATAAGGCTGCGCTTTCCGGGGCGATGGAGAGCCGGAA
ACAAAAGGAGAAAAAAGCGAAGGATGCAGAAAATAAATTAAATGAGGAAAAGAAAAAACCTC
GTAAGGGAACTAAAGATTACGGCCATGATTATTTTCCTGATCCCAAGACTGAAGATATTAAG
GGGTTGGGAGAGTTGAAAGAGGGTAAACCTAAAACCCCTAAACAAGGTGGTGGTGGTAAGCG
GGCCCGATGGTATGGTGATAAAAAGCGTAAAATTTATGAATGGGATTCCCAGCACGGTGAGC
TTGAAGGGTACCGCGCCAGTGATGGCGAACACCTCGGGGCATTCGATCCAAAAACGGGTAAG
CAGGTTAAAGGGCCGGATCCAAAACGAAACATTAAAAAATATCTTTAA
GAGGTAAGTATGGGACTTAAATTAAATTTAACCTGGTTTGATAAGAAAACCGAAGAGTTTAA
AGGTGGTGAATACTCAAAAGACTTCGGTGATGATGGTTCTGTCATTGAAAGTCTGGGGATGC
CTTTAAAGGATAATATTAATAATGGTTGGTTTGATGTTGAAAAATCATGGGTTTCGATATTA
CAGCCACACTTTAAAAATGTAATCGATATTAGTAAATTTGATTACTTTGTATCATTTGATTA
TCGGGATGGTAACTGGTAA
7. P781 (P628 TD RD-Flexible linker-P774 FL):
Amino acid sequence of P781 (SEQ ID NO: 13):
MSGGDGRGPGNSGLGHNGGQASGNVNGTSGKGGPSSGGGTDPNSGPGWGTTHTPNGDIHNYN
PGEFGNGGSKPGGNGGNSGNHSGSSGGGQSSATAMAFGLPALATPGAEGLALSVSGDALSAA
VADVLAALKGPFKFGLWGIAIYGVLPSEIAKDDPKMMSKIVTSLPADTVTETPVSSLPLDQA
TVSVTKRVADIVKDERQHIAVVTGRPMSVPVVDAKPTKRPGVESVSIPGLPSLQVSVPKGVP
AAKAPPKGIIAEKGDSRPAGFTAGGNSREAVIRFPKESGQKPVYVSVTDVLTPAQVKQRLEE
EKRRQQAWDAAHPEEGLKREYDKAKAELDAEDKNIATLNSRIASTEKAIPGARAAVQEADKK
VKEAEANKDDFVTYNPPHEYGSGWQDQVRYLDKDIQNQNEKLKAAQTSLNEMNESLSRDKAA
LSGAMESRKQKEKKAKDAENKLNEEKGSGSASGSETMVVVAPPTGFEPAGYGGGLESPSTPN
NSPSQGQIFLQVTLPYYQSAKFCQDSMAWLAQYVKTHGATDPLTIQVVANNIRYFLNADTNL
CHNPRQNVWEAFHSEMTGSGPAPAKYDYKSMSLKQMSGNVVTPAAAFGHYLWGNGEERYVNL
PDVGLKITPQQIPELMNIVNSGVTGNIPVDLNFNHNTYDSGGVIPASYLGNVSLRTIGSLNI
QNSGEWTYNGVVRAYNDYYDFNLGDYRGAIAESLTYLGAQFSGKPYHIAMPGEINISGAGHR
P781 Domain boundaries:
    1-320: P628 TD
321-460: P628 RD
461-467: Flexible linker
468-500: P774 TD
501-593: P774 RD
594-744: P774 KD
Number of amino acids: 744
Molecular weight: 78390
Theoretical pI: 6.31
DNA sequence of P781 (SEQ ID NO: 14):
ATGAGTGGTGGAGACGGACGAGGTCCGGGTAATTCAGGTCTGGGACACAATGGTGGTCAGGC
CAGTGGGAATGTGAACGGTACATCAGGTAAAGGTGGCCCTTCATCAGGTGGTGGTACGGATC
CAAACAGCGGGCCGGGCTGGGGTACGACGCATACACCTAACGGGGATATTCATAACTATAAC
CCGGGGGAGTTTGGTAACGGCGGGAGTAAACCCGGTGGTAATGGCGGTAACAGCGGCAATCA
TAGCGGGAGCTCTGGTGGCGGACAGTCTTCGGCCACCGCGATGGCCTTCGGTCTGCCTGCCT
TGGCCACTCCGGGGGCTGAAGGACTGGCTTTATCCGTTTCCGGCGATGCGTTGTCGGCCGCT
GTTGCTGATGTGCTGGCTGCCCTGAAAGGTCCGTTTAAGTTTGGTCTGTGGGGGATTGCGAT
TTACGGAGTGCTGCCTTCTGAGATAGCAAAAGATGATCCGAAAATGATGTCAAAAATCGTGA
CGTCATTGCCGGCAGACACGGTAACGGAGACGCCGGTCAGCTCCCTGCCGCTGGACCAGGCG
ACGGTCAGCGTCACTAAACGTGTGGCGGATATTGTGAAGGACGAGCGGCAGCATATTGCGGT
TGTCACCGGCCGGCCAATGAGTGTTCCTGTGGTGGATGCGAAACCGACAAAACGTCCGGGGG
TATTCAGTGTGTCGATTCCGGGTCTCCCGTCTCTGCAGGTGAGCGTACCTAAAGGTGTTCCG
GCCGCGAAAGCCCCGCCAAAAGGCATTATTGCTGAAAAAGGTGATTCACGTCCGGCTGGTTT
TACGGCCGGTGGTAACTCCCGTGAAGCCGTTATTCGTTTCCCGAAAGAGAGCGGACAGAAAC
CGGTCTATGTGTCGGTGACGGATGTTCTTACTCCGGCACAGGTAAAACAGCGTCTGGAGGAA
GAAAAGCGTCGCCAGCAGGCATGGGACGCTGCTCACCCGGAAGAGGGGCTGAAAAGAGAGTA
TGATAAAGCGAAAGCTGAGCTGGATGCTGAAGATAAAAATATTGCGACCTTAAACAGTCGCA
TTGCATCGACAGAGAAGGCGATCCCCGGTGCAAGGGCTGCTGTTCAGGAAGCCGATAAAAAG
GTAAAAGAGGCAGAGGCAAATAAAGATGATTTTGTGACTTATAACCCGCCTCATGAATATGG
CTCCGGGTGGCAGGATCAGGTTCGCTATCTTGATAAGGATATTCAGAATCAGAATGAGAAAT
TAAAAGCTGCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGCAGAAAATAA
ATTAAATGAGGAAAAGGGCTCCGGATCGGCTTCTGGGAGCGAGACCATGGTTGTTGTTGCGC
CGCCGACCGGTTTTGAGCCGGCGGGTTACGGTGGTGGCCTGTTCAGCCCGAGCACCCCGAAC
AACAGCCCGAGCCAGGGTCAAATCTTCCTGCAAGTGACCCTGCCGTACTATCAGAGCGCGAA
GTTTTGCCAAGACAGCATGGCGTGGCTGGCGCAGTACGTTAAAACCCACGGCGCGACCGATC
CGCTGACCATCCAAGTGGTTGCGAACAACATTCGTTACTTCCTGAACGCGGACACCAACCTG
TGCCACAACCCGCGTCAGAACGTGTGGGAGGCGTTTCACAGCGAAATGACCGGTAGCGGTCC
GGCGCCGGCGAAGTACGATTATAAAAGCATGAGCCTGAAGCAAATGAGCGGTAACGTGGTTA
CCCCGGCGGCGGCGTTTGGTCACTACCTGTGGGGTAACGGCGAGGAACGTTATGTGAACCTG
CCGGACGTTGGTCTGAAAATCACCCCGCAGCAAATTCCGGAGCTGATGAACATCGTGAACAG
CGGTGTTACCGGCAACATTCCGGTTGACCTGAACTTTAACCACAACACCTACGATAGCGGTG
GCGTGATCCCGGCGAGCTATCTGGGTAACGTTAGCCTGCGTACCATCGGCAGCCTGAACATT
CAGAACAGCGGCGAGTGGACCTACAACGGCGTGGTTCGTGCGTATAACGACTACTATGATTT
CAACCTGGGTGATTACCGTGGCGCGATTGCGGAAAGCCTGACCTATCTGGGCGCGCAATTTA
GCGGTAAGCCGTATCACATTGCGATGCCGGGCGAAATCAACATTAGCGGTGCGGGTCATCGT
TAA
8. P782 (P774 TD RD-Flexible linker-P628 FL):
Amino acid sequence of P782 (SEQ ID NO: 15):
MSETMVVVAPPTGFEPAGYGGGLFSPSTPNNSPSQGQIFLQVTLPYYQSAKFCQDSMAWLAQ
YVKTHGATDPLTIQVVANNIRYFLNADTNLCHNPRQNVWEAFHSEMTGSGPAPAKYDYKSMS
LKQGSGSASGSGGDGRGPGNSGLGHNGGQASGNVNGTSGKGGPSSGGGTDPNSGPGWGTTHT
PNGDIHNYNPGEFGNGGSKPGGNGGNSGNHSGSSGGGQSSATAMAFGLPALATPGAEGLALS
VSGDALSAAVADVLAALKGPFKFGLWGIAIYGVLPSEIAKDDPKMMSKIVTSLPADTVTETP
VSSLPLDQATVSVTKRVADIVKDERQHIAVVTGRPMSVPVVDAKPTKRPGVFSVSIPGLPSL
QVSVPKGVPAAKAPPKGIIAEKGDSRPAGFTAGGNSREAVIRFPKESGQKPVYVSVTDVLTP
AQVKQRLEEEKRRQQAWDAAHPEEGLKREYDKAKAELDAEDKNIATLNSRIASTEKAIPGAR
AAVQEADKKVKEAEANKDDFVTYNPPHEYGSGWQDQVRYLDKDIQNQNEKLKAAQTSLNEMN
ESLSRDKAALSGAMESRKQKEKKAKDAENKLNEEKKKPRKGTKDYGHDYFPDPKTEDIKGLG
ELKEGKPKTPKQGGGGKRARWYGDKKRKIYEWDSQHGELEGYRASDGEHLGAFDPKTGKQVK
GPDPKRNIKKYL
EVSMGLKLNLTWFDKKTEEFKGGEYSKDFGDDGSVIESLGMPLKDNINNGWFDVEKSWVSIL
QPHFKNVIDISKFDYFVSFDYRDGNW
P782 Domain boundaries:
      1-34: P774 TD
  35-127: P774 RD
128-134: flexible linker
135-453: P628 TD
454-593: P628 RD
594-694: P628 KD
695-782: P628 Immunity
Number of amino acids: 694
Molecular weight: 73546
Theoretical pI: 9.02
DNA sequence of P782 (SEQ ID NO: 16):
ATGAGCGAGACCATGGTTGTTGTTGCGCCGCCGACCGGTTTTGAGCCGGCGGGTTACGGTGG
TGGCCTGTTCAGCCCGAGCACCCCGAACAACAGCCCGAGCCAGGGTCAAATCTTCCTGCAAG
TGACCCTGCCGTACTATCAGAGCGCGAAGTTTTGCCAAGACAGCATGGCGTGGCTGGCGCAG
TACGTTAAAACCCACGGCGCGACCGATCCGCTGACCATCCAAGTGGTTGCGAACAACATTCG
TTACTTCCTGAACGCGGACACCAACCTGTGCCACAACCCGCGTCAGAACGTGTGGGAGGCGT
TTCACAGCGAAATGACCGGTAGCGGTCCGGCGCCGGCGAAGTACGATTATAAAAGCATGAGC
CTGAAGCAAGGCTCCGGATCGGCTTCTGGGAGTGGTGGAGACGGACGAGGTCCGGGTAATTC
AGGTCTGGGACACAATGGTGGTCAGGCCAGTGGGAATGTGAACGGTACATCAGGTAAAGGTG
GCCCTTCATCAGGTGGTGGTACGGATCCAAACAGCGGGCCGGGCTGGGGTACGACGCATACA
CCTAACGGGGATATTCATAACTATAACCCGGGGGAGTTTGGTAACGGCGGGAGTAAACCCGG
TGGTAATGGCGGTAACAGCGGCAATCATAGCGGGAGCTCTGGTGGCGGACAGTCTTCGGCCA
CCGCGATGGCCTTCGGTCTGCCTGCCTTGGCCACTCCGGGGGCTGAAGGACTGGCTTTATCC
GTTTCCGGCGATGCGTTGTCGGCCGCTGTTGCTGATGTGCTGGCTGCCCTGAAAGGTCCGTT
TAAGTTTGGTCTGTGGGGGATTGCGATTTACGGAGTGCTGCCTTCTGAGATAGCAAAAGATG
ATCCGAAAATGATGTCAAAAATCGTGACGTCATTGCCGGCAGACACGGTAACGGAGACGCCG
GTCAGCTCCCTGCCGCTGGACCAGGCGACGGTCAGCGTCACTAAACGTGTGGCGGATATTGT
GAAGGACGAGCGGCAGCATATTGCGGTTGTCACCGGCCGGCCAATGAGTGTTCCTGTGGTGG
ATGCGAAACCGACAAAACGTCCGGGGGTATTCAGTGTGTCGATTCCGGGTCTCCCGTCTCTG
CAGGTGAGCGTACCTAAAGGTGTTCCGGCCGCGAAAGCCCCGCCAAAAGGCATTATTGCTGA
AAAAGGTGATTCACGTCCGGCTGGTTTTACGGCCGGTGGTAACTCCCGTGAAGCCGTTATTC
GTTTCCCGAAAGAGAGCGGACAGAAACCGGTCTATGTGTCGGTGACGGATGTTCTTACTCCG
GCACAGGTAAAACAGCGTCTGGAGGAAGAAAAGCGTCGCCAGCAGGCATGGGACGCTGCTCA
CCCGGAAGAGGGGCTGAAAAGAGAGTATGATAAAGCGAAAGCTGAGCTGGATGCTGAAGATA
AAAATATTGCGACCTTAAACAGTCGCATTGCATCGACAGAGAAGGCGATCCCCGGTGCAAGG
GCTGCTGTTCAGGAAGCCGATAAAAAGGTAAAAGAGGCAGAGGCAAATAAAGATGATTTTGT
GACTTATAACCCGCCTCATGAATATGGCTCCGGGTGGCAGGATCAGGTTCGCTATCTTGATA
AGGATATTCAGAATCAGAATGAGAAATTAAAAGCTGCCCAGACATCTTTAAACGAAATGAAT
GAATCCTTATCCAGGGATAAGGCTGCGCTTTCCGGGGCGATGGAGAGCCGGAAACAAAAGGA
GAAAAAAGCGAAGGATGCAGAAAATAAATTAAATGAGGAAAAGAAAAAACCTCGTAAGGGAA
CTAAAGATTACGGCCATGATTATTTTCCTGATCCCAAGACTGAAGATATTAAGGGGTTGGGA
GAGTTGAAAGAGGGTAAACCTAAAACCCCTAAACAAGGTGGTGGTGGTAAGCGGGCCCGATG
GTATGGTGATAAAAAGCGTAAAATTTATGAATGGGATTCCCAGCACGGTGAGCTTGAAGGGT
ACCGCGCCAGTGATGGCGAACACCTCGGGGCATTCGATCCAAAAACGGGTAAGCAGGTTAAA
GGGCCGGATCCAAAACGAAACATTAAAAAATATCTTTAA
GAGGTAAGTATGGGACTTAAATTAAATTTAACCTGGTTTGATAAGAAAACCGAAGAGTTTAA
AGGTGGTGAATACTCAAAAGACTTCGGTGATGATGGTTCTGTCATTGAAAGTCTGGGGATGC
CTTTAAAGGATAATATTAATAATGGTTGGTTTGATGTTGAAAAATCATGGGTTTCGATATTA
CAGCCACACTTTAAAAATGTAATCGATATTAGTAAATTTGATTACTTTGTATCATTTGATTA
TCGGGATGGTAACTGGTAA
9. P784 (P628 TD RD-Rigid linker-P774 FL):
Amino acid sequence of P784 (SEQ ID NO: 17):
MSGGDGRGPGNSGLGHNGGQASGNVNGTSGKGGPSSGGGTDPNSGPGWGTTHTPNGDIHNYN
PGEFGNGGSKPGGNGGNSGNHSGSSGGGQSSATAMAFGLPALATPGAEGLALSVSGDALSAA
VADVLAALKGPFKFGLWGIAIYGVLPSEIAKDDPKMMSKIVTSLPADTVTETPVSSLPLDQA
TVSVTKRVADIVKDERQHIAVVTGRPMSVPVVDAKPTKRPGVESVSIPGLPSLQVSVPKGVP
AAKAPPKGIIAEKGDSRPAGFTAGGNSREAVIRFPKESGQKPVYVSVTDVLTPAQVKQRLEE
EKRRQQAWDAAHPEEGLKREYDKAKAELDAEDKNIATLNSRIASTEKAIPGARAAVQEADKK
VKEAEANKDDFVTYNPPHEYGSGWQDQVRYLDKDIQNQNEKLKAAQTSLNEMNESLSRDKAA
LSGAMESRKQKEKKAKDAENKLNEEKEAAAKEAAAKSETMVVVAPPTGFEPAGYGGGLESPS
TPNNSPSQGQIFLQVTLPYYQSAKFCQDSMAWLAQYVKTHGATDPLTIQVVANNIRYFLNAD
TNLCHNPRQNVWEAFHSEMTGSGPAPAKYDYKSMSLKQMSGNVVTPAAAFGHYLWGNGEERY
VNLPDVGLKITPQQIPELMNIVNSGVTGNIPVDLNFNHNTYDSGGVIPASYLGNVSLRTIGS
LNIQNSGEWTYNGVVRAYNDYYDFNLGDYRGAIAESLTYLGAQFSGKPYHIAMPGEINISGA
GHR
P784 Domain boundaries:
    1-320: P628 TD
321-460: P628 RD
461-470: Rigid linker
471-503: P774 TD
504-596: P774 RD
597-747: P774 KD
Number of amino acids: 747
Molecular weight: 78827
Theoretical pI: 6.31
DNA sequence of P784 (SEQ ID NO: 18):
ATGAGTGGTGGAGACGGACGAGGTCCGGGTAATTCAGGTCTGGGACACAATGGTGGTCAGGC
CAGTGGGAATGTGAACGGTACATCAGGTAAAGGTGGCCCTTCATCAGGTGGTGGTACGGATC
CAAACAGCGGGCCGGGCTGGGGTACGACGCATACACCTAACGGGGATATTCATAACTATAAC
CCGGGGGAGTTTGGTAACGGCGGGAGTAAACCCGGTGGTAATGGCGGTAACAGCGGCAATCA
TAGCGGGAGCTCTGGTGGCGGACAGTCTTCGGCCACCGCGATGGCCTTCGGTCTGCCTGCCT
TGGCCACTCCGGGGGCTGAAGGACTGGCTTTATCCGTTTCCGGCGATGCGTTGTCGGCCGCT
GTTGCTGATGTGCTGGCTGCCCTGAAAGGTCCGTTTAAGTTTGGTCTGTGGGGGATTGCGAT
TTACGGAGTGCTGCCTTCTGAGATAGCAAAAGATGATCCGAAAATGATGTCAAAAATCGTGA
CGTCATTGCCGGCAGACACGGTAACGGAGACGCCGGTCAGCTCCCTGCCGCTGGACCAGGCG
ACGGTCAGCGTCACTAAACGTGTGGCGGATATTGTGAAGGACGAGCGGCAGCATATTGCGGT
TGTCACCGGCCGGCCAATGAGTGTTCCTGTGGTGGATGCGAAACCGACAAAACGTCCGGGGG
TATTCAGTGTGTCGATTCCGGGTCTCCCGTCTCTGCAGGTGAGCGTACCTAAAGGTGTTCCG
GCCGCGAAAGCCCCGCCAAAAGGCATTATTGCTGAAAAAGGTGATTCACGTCCGGCTGGTTT
TACGGCCGGTGGTAACTCCCGTGAAGCCGTTATTCGTTTCCCGAAAGAGAGCGGACAGAAAC
CGGTCTATGTGTCGGTGACGGATGTTCTTACTCCGGCACAGGTAAAACAGCGTCTGGAGGAA
GAAAAGCGTCGCCAGCAGGCATGGGACGCTGCTCACCCGGAAGAGGGGCTGAAAAGAGAGTA
TGATAAAGCGAAAGCTGAGCTGGATGCTGAAGATAAAAATATTGCGACCTTAAACAGTCGCA
TTGCATCGACAGAGAAGGCGATCCCCGGTGCAAGGGCTGCTGTTCAGGAAGCCGATAAAAAG
GTAAAAGAGGCAGAGGCAAATAAAGATGATTTTGTGACTTATAACCCGCCTCATGAATATGG
CTCCGGGTGGCAGGATCAGGTTCGCTATCTTGATAAGGATATTCAGAATCAGAATGAGAAAT
TAAAAGCTGCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGCAGAAAATAA
ATTAAATGAGGAAAAGGAAGCCGCTGCCAAGGAGGCGGCCGCTAAAAGCGAGACCATGGTTG
TTGTTGCGCCGCCGACCGGTTTTGAGCCGGCGGGTTACGGTGGTGGCCTGTTCAGCCCGAGC
ACCCCGAACAACAGCCCGAGCCAGGGTCAAATCTTCCTGCAAGTGACCCTGCCGTACTATCA
GAGCGCGAAGTTTTGCCAAGACAGCATGGCGTGGCTGGCGCAGTACGTTAAAACCCACGGCG
CGACCGATCCGCTGACCATCCAAGTGGTTGCGAACAACATTCGTTACTTCCTGAACGCGGAC
ACCAACCTGTGCCACAACCCGCGTCAGAACGTGTGGGAGGCGTTTCACAGCGAAATGACCGG
TAGCGGTCCGGCGCCGGCGAAGTACGATTATAAAAGCATGAGCCTGAAGCAAATGAGCGGTA
ACGTGGTTACCCCGGCGGCGGCGTTTGGTCACTACCTGTGGGGTAACGGCGAGGAACGTTAT
GTGAACCTGCCGGACGTTGGTCTGAAAATCACCCCGCAGCAAATTCCGGAGCTGATGAACAT
CGTGAACAGCGGTGTTACCGGCAACATTCCGGTTGACCTGAACTTTAACCACAACACCTACG
ATAGCGGTGGCGTGATCCCGGCGAGCTATCTGGGTAACGTTAGCCTGCGTACCATCGGCAGC
CTGAACATTCAGAACAGCGGCGAGTGGACCTACAACGGCGTGGTTCGTGCGTATAACGACTA
CTATGATTTCAACCTGGGTGATTACCGTGGCGCGATTGCGGAAAGCCTGACCTATCTGGGCG
CGCAATTTAGCGGTAAGCCGTATCACATTGCGATGCCGGGCGAAATCAACATTAGCGGTGCG
GGTCATCGTTAA
10. P787 (P764 TD RD-Flexible linker-P774 FL):
Amino acid sequence of P789 (SEQ ID NO: 19):
MPEETLTVVGGGNNSCNVSWGGGNGNNGGAGYSGKYGGTSYEGATSMLKLNDRVLIQLYLCN
PLNPDYIGAPWGSDKDAESIIRANRDKAGKFKANIQNWKTSGTGSLGSPVVGKSYSSGDVDT
YSVSFGKEKYNVLYNRKKDSFTTAYVDGGANKPEHSGSGSASGSETMVVVAPPTGFEPAGYG
GGLFSPSTPNNSPSQGQIFLQVTLPYYQSAKFCQDSMAWLAQYVKTHGATDPLTIQVVANNI
RYFLNADTNLCHNPRQNVWEAFHSEMTGSGPAPAKYDYKSMSLKQMSGNVVTPAAAFGHYLW
GNGEERYVNLPDVGLKITPQQIPELMNIVNSGVTGNIPVDLNFNHNTYDSGGVIPASYLGNV
SLRTIGSLNIQNSGEWTYNGVVRAYNDYYDFNLGDYRGAIAESLTYLGAQFSGKPYHIAMPG
EINISGAGHR
P787 Domain boundaries:
1-38: P764 TD
  39-160: P764 RD
161-167: Flexible linker
168-200: P774 TD
201-293: P774 RD
294-444: P774 KD
Number of amino acids: 444
Molecular weight: 47574
Theoretical pI: 6.07
DNA sequence of P787 (SEQ ID NO: 20):
ATGCCTGAAGAAACATTGACTGTCGTAGGTGGTGGTAATAACAGCTGTAATGTTAGCTGGGG
TGGTGGCAATGGTAACAACGGTGGTGCTGGCTATTCTGGTAAATACGGTGGTACTAGCTATG
AAGGTGCAACTAGTATGTTGAAGTTGAATGACCGTGTTTTAATTCAGCTTTATCTTTGCAAT
CCGCTTAACCCAGATTATATTGGAGCACCTTGGGGCAGTGATAAAGATGCAGAATCAATTAT
CAGAGCTAACAGAGATAAAGCAGGAAAGTTCAAGGCTAATATTCAGAACTGGAAAACAAGCG
GTACAGGTTCTTTAGGTAGCCCTGTAGTTGGGAAATCTTATAGCTCAGGTGATGTAGATACT
TATTCGGTTAGCTTCGGAAAAGAAAAATACAATGTCCTGTATAACCGTAAAAAAGACTCTTT
TACTACAGCTTATGTCGATGGCGGCGCAAATAAACCTGAGCATGGCTCCGGATCGGCTTCTG
GGAGCGAGACCATGGTTGTTGTTGCGCCGCCGACCGGTTTTGAGCCGGCGGGTTACGGTGGT
GGCCTGTTCAGCCCGAGCACCCCGAACAACAGCCCGAGCCAGGGTCAAATCTTCCTGCAAGT
GACCCTGCCGTACTATCAGAGCGCGAAGTTTTGCCAAGACAGCATGGCGTGGCTGGCGCAGT
ACGTTAAAACCCACGGCGCGACCGATCCGCTGACCATCCAAGTGGTTGCGAACAACATTCGT
TACTTCCTGAACGCGGACACCAACCTGTGCCACAACCCGCGTCAGAACGTGTGGGAGGCGTT
TCACAGCGAAATGACCGGTAGCGGTCCGGCGCCGGCGAAGTACGATTATAAAAGCATGAGCC
TGAAGCAAATGAGCGGTAACGTGGTTACCCCGGCGGCGGCGTTTGGTCACTACCTGTGGGGT
AACGGCGAGGAACGTTATGTGAACCTGCCGGACGTTGGTCTGAAAATCACCCCGCAGCAAAT
TCCGGAGCTGATGAACATCGTGAACAGCGGTGTTACCGGCAACATTCCGGTTGACCTGAACT
TTAACCACAACACCTACGATAGCGGTGGCGTGATCCCGGCGAGCTATCTGGGTAACGTTAGC
CTGCGTACCATCGGCAGCCTGAACATTCAGAACAGCGGCGAGTGGACCTACAACGGCGTGGT
TCGTGCGTATAACGACTACTATGATTTCAACCTGGGTGATTACCGTGGCGCGATTGCGGAAA
GCCTGACCTATCTGGGCGCGCAATTTAGCGGTAAGCCGTATCACATTGCGATGCCGGGCGAA
ATCAACATTAGCGGTGCGGGTCATCGTTAA
11. P789 (P764 TD RD-Rigid linker-P774 FL):
Amino acid sequence (SEQ ID NO: 21):
MPEETLTVVGGGNNSCNVSWGGGNGNNGGAGYSGKYGGTSYEGATSMLKLNDRVLIQLYLCN
PLNPDYIGAPWGSDKDAESIIRANRDKAGKFKANIQNWKTSGTGSLGSPVVGKSYSSGDVDT
YSVSFGKEKYNVLYNRKKDSFTTAYVDGGANKPEHSEAAAKEAAAKSETMVVVAPPTGFEPA
GYGGGLESPSTPNNSPSQGQIFLQVTLPYYQSAKFCQDSMAWLAQYVKTHGATDPLTIQVVA
NNIRYFLNADTNLCHNPRQNVWEAFHSEMTGSGPAPAKYDYKSMSLKQMSGNVVTPAAAFGH
YLWGNGEERYVNLPDVGLKITPQQIPELMNIVNSGVTGNIPVDLNENHNTYDSGGVIPASYL
GNVSLRTIGSLNIQNSGEWTYNGVVRAYNDYYDENLGDYRGAIAESLTYLGAQFSGKPYHIA
MPGEINISGAGHR
P789 Domain boundaries:
1-38: P764 TD
  39-160: P764 RD
161-170: Rigid linker
171-203: P774 TD
204-296: P774 RD
297-447: P774 KD
Number of amino acids: 447
Molecular weight: 48012
Theoretical pI: 6.08
DNA sequence of P789 (SEQ ID NO: 22):
ATGCCTGAAGAAACATTGACTGTCGTAGGTGGTGGTAATAACAGCTGTAATGTTAGCTGGGG
TGGTGGCAATGGTAACAACGGTGGTGCTGGCTATTCTGGTAAATACGGTGGTACTAGCTATG
AAGGTGCAACTAGTATGTTGAAGTTGAATGACCGTGTTTTAATTCAGCTTTATCTTTGCAAT
CCGCTTAACCCAGATTATATTGGAGCACCTTGGGGCAGTGATAAAGATGCAGAATCAATTAT
CAGAGCTAACAGAGATAAAGCAGGAAAGTTCAAGGCTAATATTCAGAACTGGAAAACAAGCG
GTACAGGTTCTTTAGGTAGCCCTGTAGTTGGGAAATCTTATAGCTCAGGTGATGTAGATACT
TATTCGGTTAGCTTCGGAAAAGAAAAATACAATGTCCTGTATAACCGTAAAAAAGACTCTTT
TACTACAGCTTATGTCGATGGCGGCGCAAATAAACCTGAGCATGAAGCCGCTGCCAAGGAGG
CGGCCGCTAAAAGCGAGACCATGGTTGTTGTTGCGCCGCCGACCGGTTTTGAGCCGGCGGGT
TACGGTGGTGGCCTGTTCAGCCCGAGCACCCCGAACAACAGCCCGAGCCAGGGTCAAATCTT
CCTGCAAGTGACCCTGCCGTACTATCAGAGCGCGAAGTTTTGCCAAGACAGCATGGCGTGGC
TGGCGCAGTACGTTAAAACCCACGGCGCGACCGATCCGCTGACCATCCAAGTGGTTGCGAAC
AACATTCGTTACTTCCTGAACGCGGACACCAACCTGTGCCACAACCCGCGTCAGAACGTGTG
GGAGGCGTTTCACAGCGAAATGACCGGTAGCGGTCCGGCGCCGGCGAAGTACGATTATAAAA
GCATGAGCCTGAAGCAAATGAGCGGTAACGTGGTTACCCCGGCGGCGGCGTTTGGTCACTAC
CTGTGGGGTAACGGCGAGGAACGTTATGTGAACCTGCCGGACGTTGGTCTGAAAATCACCCC
GCAGCAAATTCCGGAGCTGATGAACATCGTGAACAGCGGTGTTACCGGCAACATTCCGGTTG
ACCTGAACTTTAACCACAACACCTACGATAGCGGTGGCGTGATCCCGGCGAGCTATCTGGGT
AACGTTAGCCTGCGTACCATCGGCAGCCTGAACATTCAGAACAGCGGCGAGTGGACCTACAA
CGGCGTGGTTCGTGCGTATAACGACTACTATGATTTCAACCTGGGTGATTACCGTGGCGCGA
TTGCGGAAAGCCTGACCTATCTGGGCGCGCAATTTAGCGGTAAGCCGTATCACATTGCGATG
CCGGGCGAAATCAACATTAGCGGTGCGGGTCATCGTTAA
12. P788 (P764 TD-Rigid linker-P628 FL):
Amino acid sequence of P788 (SEQ ID NO: 23):
MPEETLTVVGGGNNSCNVSWGGGNGNNGGAGYSGKYGGEAAAKEAAAKSGGDGRGPGNSGLG
HNGGQASGNVNGTSGKGGPSSGGGTDPNSGPGWGTTHTPNGDIHNYNPGEFGNGGSKPGGNG
GNSGNHSGSSGGGQSSATAMAFGLPALATPGAEGLALSVSGDALSAAVADVLAALKGPFKFG
LWGIAIYGVLPSEIAKDDPKMMSKIVTSLPADTVTETPVSSLPLDQATVSVTKRVADIVKDE
RQHIAVVTGRPMSVPVVDAKPTKRPGVFSVSIPGLPSLQVSVPKGVPAAKAPPKGIIAEKGD
SRPAGFTAGGNSREAVIRFPKESGQKPVYVSVTDVLTPAQVKQRLEEEKRRQQAWDAAHPEE
GLKREYDKAKAELDAEDKNIATLNSRIASTEKAIPGARAAVQEADKKVKEAEANKDDEVTYN
PPHEYGSGWQDQVRYLDKDIQNQNEKLKAAQTSLNEMNESLSRDKAALSGAMESRKQKEKKA
KDAENKLNEEKKKPRKGTKDYGHDYFPDPKTEDIKGLGELKEGKPKTPKQGGGGKRARWYGD
KKRKIYEWDSQHGELEGYRASDGEHLGAFDPKTGKQVKGPDPKRNIKKYL
EVSMGLKLNLTWFDKKTEEFKGGEYSKDFGDDGSVIESLGMPLKDNINNGWEDVEKSWVSIL
QPHFKNVIDISKFDYFVSFDYRDGNW
P788 Domain boundaries:
      1-38: P764 TD
    39-48: Rigid linker
  49-367: P628 TD
368-507: P628 RD
508-608: P628 KD
609-696: P628 Immunity
Number of amino acids: 608
Molecular weight: 63648
Theoretical pI: 9.09
DNA sequence of P788 (SEQ ID NO: 24):
ATGCCTGAAGAAACATTGACTGTCGTAGGTGGTGGTAATAACAGCTGTAATGTTAGCTGGGGTGGTGGCAATGGTAAC
AACGGTGGTGCTGGCTATTCTGGTAAATACGGTGGTGAAGCCGCTGCCAAGGAGGCGGCCGCTAAAAGTG
GTGGAGACGGACGAGGTCCGGGTAATTCAGGTCTGGGACACAATGGTGGTCAGGCCAGTGGG
AATGTGAACGGTACATCAGGTAAAGGTGGCCCTTCATCAGGTGGTGGTACGGATCCAAACAG
CGGGCCGGGCTGGGGTACGACGCATACACCTAACGGGGATATTCATAACTATAACCCGGGGG
AGTTTGGTAACGGCGGGAGTAAACCCGGTGGTAATGGCGGTAACAGCGGCAATCATAGCGGG
AGCTCTGGTGGCGGACAGTCTTCGGCCACCGCGATGGCCTTCGGTCTGCCTGCCTTGGCCAC
TCCGGGGGCTGAAGGACTGGCTTTATCCGTTTCCGGCGATGCGTTGTCGGCCGCTGTTGCTG
ATGTGCTGGCTGCCCTGAAAGGTCCGTTTAAGTTTGGTCTGTGGGGGATTGCGATTTACGGA
GTGCTGCCTTCTGAGATAGCAAAAGATGATCCGAAAATGATGTCAAAAATCGTGACGTCATT
GCCGGCAGACACGGTAACGGAGACGCCGGTCAGCTCCCTGCCGCTGGACCAGGCGACGGTCA
GCGTCACTAAACGTGTGGCGGATATTGTGAAGGACGAGCGGCAGCATATTGCGGTTGTCACC
GGCCGGCCAATGAGTGTTCCTGTGGTGGATGCGAAACCGACAAAACGTCCGGGGGTATTCAG
TGTGTCGATTCCGGGTCTCCCGTCTCTGCAGGTGAGCGTACCTAAAGGTGTTCCGGCCGCGA
AAGCCCCGCCAAAAGGCATTATTGCTGAAAAAGGTGATTCACGTCCGGCTGGTTTTACGGCC
GGTGGTAACTCCCGTGAAGCCGTTATTCGTTTCCCGAAAGAGAGCGGACAGAAACCGGTCTA
TGTGTCGGTGACGGATGTTCTTACTCCGGCACAGGTAAAACAGCGTCTGGAGGAAGAAAAGC
GTCGCCAGCAGGCATGGGACGCTGCTCACCCGGAAGAGGGGCTGAAAAGAGAGTATGATAAA
GCGAAAGCTGAGCTGGATGCTGAAGATAAAAATATTGCGACCTTAAACAGTCGCATTGCATC
GACAGAGAAGGCGATCCCCGGTGCAAGGGCTGCTGTTCAGGAAGCCGATAAAAAGGTAAAAG
AGGCAGAGGCAAATAAAGATGATTTTGTGACTTATAACCCGCCTCATGAATATGGCTCCGGG
TGGCAGGATCAGGTTCGCTATCTTGATAAGGATATTCAGAATCAGAATGAGAAATTAAAAGC
TGCCCAGACATCTTTAAACGAAATGAATGAATCCTTATCCAGGGATAAGGCTGCGCTTTCCG
GGGCGATGGAGAGCCGGAAACAAAAGGAGAAAAAAGCGAAGGATGCAGAAAATAAATTAAAT
GAGGAAAAGAAAAAACCTCGTAAGGGAACTAAAGATTACGGCCATGATTATTTTCCTGATCC
CAAGACTGAAGATATTAAGGGGTTGGGAGAGTTGAAAGAGGGTAAACCTAAAACCCCTAAAC
AAGGTGGTGGTGGTAAGCGGGCCCGATGGTATGGTGATAAAAAGCGTAAAATTTATGAATGG
GATTCCCAGCACGGTGAGCTTGAAGGGTACCGCGCCAGTGATGGCGAACACCTCGGGGCATT
CGATCCAAAAACGGGTAAGCAGGTTAAAGGGCCGGATCCAAAACGAAACATTA
AAAAATATCTTTAA
GAGGTAAGTATGGGACTTAAATTAAATTTAACCTGGTTTGATAAGAAAACCGAAGAGTTTAA
AGGTGGTGAATACTCAAAAGACTTCGGTGATGATGGTTCTGTCATTGAAAGTCTGGGGATGC
CTTTAAAGGATAATATTAATAATGGTTGGTTTGATGTTGAAAAATCATGGGTTTCGATATTA
CAGCCACACTTTAAAAATGTAATCGATATTAGTAAATTTGATTACTTTGTATCATTTGATTA
TCGGGATGGTAACTGGTAA
13. P792 (P764-Rigid Linker-P774 FL):
Amino acid sequence of P792 (SEQ ID NO: 25):
MPEETLTVVGGGNNSCNVSWGGGNGNNGGAGYSGKYGGTSYEGATSMLKLNDRVLIQLYLCN
PLNPDYIGAPWGSDKDAESIIRANRDKAGKFKANIQNWKTSGTGSLGSPVVGKSYSSGDVDT
YSVSFGKEKYNVLYNRKKDSFTTAYVDGGANKPEHSMKDQAIAVVKLYLLNESQASVIDTTS
GIITDSGKTLSGKLGDKYNTLAKEAADNIKNFQGKKLRSENDAMASINELANNPKMKLSQAD
KTVVSNALKQMDLSALADRFKGLEKAFTWGDRLLKAEKIREGVVTGITTGDWQKLAFEVEAM
YLSGVAGAVALGITTAMISTVAVALSLPAVAVSALTVVSVIGISILTSYIDADKAKALNNAV
LGLFKEAAAKEAAAKSETMVVVAPPTGFEPAGYGGGLESPSTPNNSPSQGQIFLQVTLPYYQ
SAKFCQDSMAWLAQYVKTHGATDPLTIQVVANNIRYFLNADTNLCHNPRQNVWEAFHSEMTG
SGPAPAKYDYKSMSLKQMSGNVVTPAAAFGHYLWGNGEERYVNLPDVGLKITPQQIPELMNI
VNSGVTGNIPVDLNFNHNTYDSGGVIPASYLGNVSLRTIGSLNIQNSGEWTYNGVVRAYNDY
YDENLGDYRGAIAESLTYLGAQFSGKPYHIAMPGEINISGAGHR
P792 Domain boundaries:
      1-38: 764 TD
  39-160: 764 RD
161-377: 764 KD
378-387: Rigid linker
388-420: P774 TD
421-513: P774 RD
514-664: P774 KD
Number of amino acids: 664
Molecular weight: 70953
Theoretical pI: 7.08
DNA sequence of P792 (SEQ ID NO: 26):
ATGCCTGAAGAAACATTGACTGTCGTAGGTGGTGGTAATAACAGCTGTAATGTTAGCTGGGG
TGGTGGCAATGGTAACAACGGTGGTGCTGGCTATTCTGGTAAATACGGTGGTACTAGCTATG
AAGGTGCAACTAGTATGTTGAAGTTGAATGACCGTGTTTTAATTCAGCTTTATCTTTGCAAT
CCGCTTAACCCAGATTATATTGGAGCACCTTGGGGCAGTGATAAAGATGCAGAATCAATTAT
CAGAGCTAACAGAGATAAAGCAGGAAAGTTCAAGGCTAATATTCAGAACTGGAAAACAAGCG
GTACAGGTTCTTTAGGTAGCCCTGTAGTTGGGAAATCTTATAGCTCAGGTGATGTAGATACT
TATTCGGTTAGCTTCGGAAAAGAAAAATACAATGTCCTGTATAACCGTAAAAAAGACTCTTT
TACTACAGCTTATGTCGATGGCGGCGCAAATAAACCTGAGCATAGCATGAAGGATCAGGCTA
TTGCCGTTGTTAAACTTTACCTTCTCAACGAAAGTCAGGCATCGGTAATCGATACTACATCG
GGAATTATTACTGACTCTGGTAAAACTCTTAGCGGGAAATTAGGTGATAAATACAACACTCT
GGCGAAAGAAGCTGCTGACAATATAAAAAACTTCCAGGGTAAGAAACTCCGCAGTTTTAATG
ATGCTATGGCATCTATTAATGAACTAGCTAACAATCCAAAGATGAAGTTAAGTCAGGCGGAT
AAAACAGTCGTTTCTAATGCCCTCAAACAAATGGATTTGTCAGCACTAGCTGACCGATTCAA
AGGGTTAGAGAAAGCCTTTACTTGGGGTGATCGACTTCTTAAAGCCGAGAAAATCAGAGAAG
GTGTTGTTACTGGTATTACCACAGGGGACTGGCAAAAGCTGGCGTTTGAGGTTGAAGCTATG
TACCTCAGTGGTGTTGCTGGCGCCGTAGCGTTAGGGATTACTACTGCCATGATTAGCACAGT
CGCAGTCGCTTTGTCACTTCCAGCTGTAGCTGTCTCTGCGCTTACTGTTGTGTCCGTCATTG
GCATCTCTATTCTCACATCTTATATCGATGCTGATAAGGCCAAAGCACTGAATAATGCAGTG
CTTGGCTTATTTAAAGAAGCCGCTGCCAAGGAGGCGGCCGCTAAAAGCGAGACCATGGTTGT
TGTTGCGCCGCCGACCGGTTTTGAGCCGGCGGGTTACGGTGGTGGCCTGTTCAGCCCGAGCA
CCCCGAACAACAGCCCGAGCCAGGGTCAAATCTTCCTGCAAGTGACCCTGCCGTACTATCAG
AGCGCGAAGTTTTGCCAAGACAGCATGGCGTGGCTGGCGCAGTACGTTAAAACCCACGGCGC
GACCGATCCGCTGACCATCCAAGTGGTTGCGAACAACATTCGTTACTTCCTGAACGCGGACA
CCAACCTGTGCCACAACCCGCGTCAGAACGTGTGGGAGGCGTTTCACAGCGAAATGACCGGT
AGCGGTCCGGCGCCGGCGAAGTACGATTATAAAAGCATGAGCCTGAAGCAAATGAGCGGTAA
CGTGGTTACCCCGGCGGCGGCGTTTGGTCACTACCTGTGGGGTAACGGCGAGGAACGTTATG
TGAACCTGCCGGACGTTGGTCTGAAAATCACCCCGCAGCAAATTCCGGAGCTGATGAACATC
GTGAACAGCGGTGTTACCGGCAACATTCCGGTTGACCTGAACTTTAACCACAACACCTACGA
TAGCGGTGGCGTGATCCCGGCGAGCTATCTGGGTAACGTTAGCCTGCGTACCATCGGCAGCC
TGAACATTCAGAACAGCGGCGAGTGGACCTACAACGGCGTGGTTCGTGCGTATAACGACTAC
TATGATTTCAACCTGGGTGATTACCGTGGCGCGATTGCGGAAAGCCTGACCTATCTGGGCGC
GCAATTTAGCGGTAAGCCGTATCACATTGCGATGCCGGGCGAAATCAACATTAGCGGTGCGG
GTCATCGTTAA
14. P795 (P764-Flexible Linker-P774 FL):
Amino acid sequence of P795 (SEQ ID NO: 27):
MPEETLTVVGGGNNSCNVSWGGGNGNNGGAGYSGKYGGTSYEGATSMLKLNDRVLIQLYLCN
PLNPDYIGAPWGSDKDAESIIRANRDKAGKFKANIQNWKTSGTGSLGSPVVGKSYSSGDVDT
YSVSFGKEKYNVLYNRKKDSFTTAYVDGGANKPEHSMKDQAIAVVKLYLLNESQASVIDTTS
GIITDSGKTLSGKLGDKYNTLAKEAADNIKNFQGKKLRSFNDAMASINELANNPKMKLSQAD
KTVVSNALKQMDLSALADRFKGLEKAFTWGDRLLKAEKIREGVVTGITTGDWQKLAFEVEAM
YLSGVAGAVALGITTAMISTVAVALSLPAVAVSALTVVSVIGISILTSYIDADKAKALNNAV
LGLFKGSGSASGSETMVVVAPPTGFEPAGYGGGLESPSTPNNSPSQGQIFLQVTLPYYQSAK
FCQDSMAWLAQYVKTHGATDPLTIQVVANNIRYFLNADTNLCHNPRQNVWEAFHSEMTGSGP
APAKYDYKSMSLKQMSGNVVTPAAAFGHYLWGNGEERYVNLPDVGLKITPQQIPELMNIVNS
GVTGNIPVDLNFNHNTYDSGGVIPASYLGNVSLRTIGSLNIQNSGEWTYNGVVRAYNDYYDF
NLGDYRGAIAESLTYLGAQFSGKPYHIAMPGEINISGAGHR
P795 Domain
      1-38: 764 TD
  39-160: 764 RD
161-377: 764 KD
378-384: Rigid linker
385-417: P774 TD
418-510: P774 RD
511-661: P774 KD
Number of amino acids: 661
Molecular weight: 70516
Theoretical pI: 7.08
DNA sequence of P795 (SEQ ID NO: 28):
ATGCCTGAAGAAACATTGACTGTCGTAGGTGGTGGTAATAACAGCTGTAATGTTAGCTGGGG
TGGTGGCAATGGTAACAACGGTGGTGCTGGCTATTCTGGTAAATACGGTGGTACTAGCTATG
AAGGTGCAACTAGTATGTTGAAGTTGAATGACCGTGTTTTAATTCAGCTTTATCTTTGCAAT
CCGCTTAACCCAGATTATATTGGAGCACCTTGGGGCAGTGATAAAGATGCAGAATCAATTAT
CAGAGCTAACAGAGATAAAGCAGGAAAGTTCAAGGCTAATATTCAGAACTGGAAAACAAGCG
GTACAGGTTCTTTAGGTAGCCCTGTAGTTGGGAAATCTTATAGCTCAGGTGATGTAGATACT
TATTCGGTTAGCTTCGGAAAAGAAAAATACAATGTCCTGTATAACCGTAAAAAAGACTCTTT
TACTACAGCTTATGTCGATGGCGGCGCAAATAAACCTGAGCATAGCATGAAGGATCAGGCTA
TTGCCGTTGTTAAACTTTACCTTCTCAACGAAAGTCAGGCATCGGTAATCGATACTACATCG
GGAATTATTACTGACTCTGGTAAAACTCTTAGCGGGAAATTAGGTGATAAATACAACACTCT
GGCGAAAGAAGCTGCTGACAATATAAAAAACTTCCAGGGTAAGAAACTCCGCAGTTTTAATG
ATGCTATGGCATCTATTAATGAACTAGCTAACAATCCAAAGATGAAGTTAAGTCAGGCGGAT
AAAACAGTCGTTTCTAATGCCCTCAAACAAATGGATTTGTCAGCACTAGCTGACCGATTCAA
AGGGTTAGAGAAAGCCTTTACTTGGGGTGATCGACTTCTTAAAGCCGAGAAAATCAGAGAAG
GTGTTGTTACTGGTATTACCACAGGGGACTGGCAAAAGCTGGCGTTTGAGGTTGAAGCTATG
TACCTCAGTGGTGTTGCTGGCGCCGTAGCGTTAGGGATTACTACTGCCATGATTAGCACAGT
CGCAGTCGCTTTGTCACTTCCAGCTGTAGCTGTCTCTGCGCTTACTGTTGTGTCCGTCATTG
GCATCTCTATTCTCACATCTTATATCGATGCTGATAAGGCCAAAGCACTGAATAATGCAGTG
CTTGGCTTATTTAAAGGCTCCGGATCGGCTTCTGGGAGCGAGACCATGGTTGTTGTTGCGCC
GCCGACCGGTTTTGAGCCGGCGGGTTACGGTGGTGGCCTGTTCAGCCCGAGCACCCCGAACA
ACAGCCCGAGCCAGGGTCAAATCTTCCTGCAAGTGACCCTGCCGTACTATCAGAGCGCGAAG
TTTTGCCAAGACAGCATGGCGTGGCTGGCGCAGTACGTTAAAACCCACGGCGCGACCGATCC
GCTGACCATCCAAGTGGTTGCGAACAACATTCGTTACTTCCTGAACGCGGACACCAACCTGT
GCCACAACCCGCGTCAGAACGTGTGGGAGGCGTTTCACAGCGAAATGACCGGTAGCGGTCCG
GCGCCGGCGAAGTACGATTATAAAAGCATGAGCCTGAAGCAAATGAGCGGTAACGTGGTTAC
CCCGGCGGCGGCGTTTGGTCACTACCTGTGGGGTAACGGCGAGGAACGTTATGTGAACCTGC
CGGACGTTGGTCTGAAAATCACCCCGCAGCAAATTCCGGAGCTGATGAACATCGTGAACAGC
GGTGTTACCGGCAACATTCCGGTTGACCTGAACTTTAACCACAACACCTACGATAGCGGTGG
CGTGATCCCGGCGAGCTATCTGGGTAACGTTAGCCTGCGTACCATCGGCAGCCTGAACATTC
AGAACAGCGGCGAGTGGACCTACAACGGCGTGGTTCGTGCGTATAACGACTACTATGATTTC
AACCTGGGTGATTACCGTGGCGCGATTGCGGAAAGCCTGACCTATCTGGGCGCGCAATTTAG
CGGTAAGCCGTATCACATTGCGATGCCGGGCGAAATCAACATTAGCGGTGCGGGTCATCGTT
AA
15. P805 (P628 Δ7):
Amino acid sequence of P805 (SEQ ID NO: 29):
MGPGNSGLGHNGGQASGNVNGTSGKGGPSSGGGTDPNSGPGWGTTHTPNGDIHNYNPGEFGN
GGSKPGGNGGNSGNHSGSSGGGQSSATAMAFGLPALATPGAEGLALSVSGDALSAAVADVLA
ALKGPFKFGLWGIAIYGVLPSEIAKDDPKMMSKIVTSLPADTVTETPVSSLPLDQATVSVTK
RVADIVKDERQHIAVVTGRPMSVPVVDAKPTKRPGVESVSIPGLPSLQVSVPKGVPAAKAPP
KGIIAEKGDSRPAGFTAGGNSREAVIRFPKESGQKPVYVSVTDVLTPAQVKQRLEEEKRRQQ
AWDAAHPEEGLKREYDKAKAELDAEDKNIATLNSRIASTEKAIPGARAAVQEADKKVKEAEA
NKDDFVTYNPPHEYGSGWQDQVRYLDKDIQNQNEKLKAAQTSLNEMNESLSRDKAALSGAME
SRKQKEKKAKDAENKLNEEKKKPRKGTKDYGHDYFPDPKTEDIKGLGELKEGKPKTPKQGGG
GKRARWYGDKKRKIYEWDSQHGELEGYRASDGEHLGAFDPKTGKQVKGPDPKRNIKKYL
EVSMGLKLNLTWFDKKTEEFKGGEYSKDFGDDGSVIESLGMPLKDNINNGWFDVEKSWVSIL
QPHEKNVIDISKFDYFVSFDYRDGNW
P805 Domain boundaries:
    1-314: 628 TD
315-454: 628 RD
455-555: 628 KD
556-643: 628 Immunity
Number of amino acids: 555
Molecular weight: 58707
Theoretical pI: 9.21
DNA sequence of P805 (SEQ ID NO: 30):
ATGGGTCCGGGTAATTCAGGTCTGGGACACAATGGTGGTCAGGCCAGTGGGAATGTGAACGG
TACATCAGGTAAAGGTGGCCCTTCATCAGGTGGTGGTACGGATCCAAACAGCGGGCCGGGCT
GGGGTACGACGCATACACCTAACGGGGATATTCATAACTATAACCCGGGGGAGTTTGGTAAC
GGCGGGAGTAAACCCGGTGGTAATGGCGGTAACAGCGGCAATCATAGCGGGAGCTCTGGTGG
CGGACAGTCTTCGGCCACCGCGATGGCCTTCGGTCTGCCTGCCTTGGCCACTCCGGGGGCTG
AAGGACTGGCTTTATCCGTTTCCGGCGATGCGTTGTCGGCCGCTGTTGCTGATGTGCTGGCT
GCCCTGAAAGGTCCGTTTAAGTTTGGTCTGTGGGGGATTGCGATTTACGGAGTGCTGCCTTC
TGAGATAGCAAAAGATGATCCGAAAATGATGTCAAAAATCGTGACGTCATTGCCGGCAGACA
CGGTAACGGAGACGCCGGTCAGCTCCCTGCCGCTGGACCAGGCGACGGTCAGCGTCACTAAA
CGTGTGGCGGATATTGTGAAGGACGAGCGGCAGCATATTGCGGTTGTCACCGGCCGGCCAAT
GAGTGTTCCTGTGGTGGATGCGAAACCGACAAAACGTCCGGGGGTATTCAGTGTGTCGATTC
CGGGTCTCCCGTCTCTGCAGGTGAGCGTACCTAAAGGTGTTCCGGCCGCGAAAGCCCCGCCA
AAAGGCATTATTGCTGAAAAAGGTGATTCACGTCCGGCTGGTTTTACGGCCGGTGGTAACTC
CCGTGAAGCCGTTATTCGTTTCCCGAAAGAGAGCGGACAGAAACCGGTCTATGTGTCGGTGA
CGGATGTTCTTACTCCGGCACAGGTAAAACAGCGTCTGGAGGAAGAAAAGCGTCGCCAGCAG
GCATGGGACGCTGCTCACCCGGAAGAGGGGCTGAAAAGAGAGTATGATAAAGCGAAAGCTGA
GCTGGATGCTGAAGATAAAAATATTGCGACCTTAAACAGTCGCATTGCATCGACAGAGAAGG
CGATCCCCGGTGCAAGGGCTGCTGTTCAGGAAGCCGATAAAAAGGTAAAAGAGGCAGAGGCA
AATAAAGATGATTTTGTGACTTATAACCCGCCTCATGAATATGGCTCCGGGTGGCAGGATCA
GGTTCGCTATCTTGATAAGGATATTCAGAATCAGAATGAGAAATTAAAAGCTGCCCAGACAT
CTTTAAACGAAATGAATGAATCCTTATCCAGGGATAAGGCTGCGCTTTCCGGGGCGATGGAG
AGCCGGAAACAAAAGGAGAAAAAAGCGAAGGATGCAGAAAATAAATTAAATGAGGAAAAGAA
AAAACCTCGTAAGGGAACTAAAGATTACGGCCATGATTATTTTCCTGATCCCAAGACTGAAG
ATATTAAGGGGTTGGGAGAGTTGAAAGAGGGTAAACCTAAAACCCCTAAACAAGGTGGTGGT
GGTAAGCGGGCCCGATGGTATGGTGATAAAAAGCGTAAAATTTATGAATGGGATTCCCAGCA
CGGTGAGCTTGAAGGGTACCGCGCCAGTGATGGCGAACACCTCGGGGCATTCGATCCAAAAA
CGGGTAAGCAGGTTAAAGGGCCGGATCCAAAACGAAACATTA AAAAATATCTTTAA
GAGGTAAGTATGGGACTTAAATTAAATTTAACCTGGTTTGATAAGAAAACCGAAGAGTTTAA
AGGTGGTGAATACTCAAAAGACTTCGGTGATGATGGTTCTGTCATTGAAAGTCTGGGGATGC
CTTTAAAGGATAATATTAATAATGGTTGGTTTGATGTTGAAAAATCATGGGTTTCGATATTA
CAGCCACACTTTAAAAATGTAATCGATATTAGTAAATTTGATTACTTTGTATCATTTGATTA
TCGGGATGGTAACTGGTAA
16. P806 (P628 Δ25):
Amino acid sequence of P806 (SEQ ID NO: 31):
MNGTSGKGGPSSGGGTDPNSGPGWGTTHTPNGDIHNYNPGEFGNGGSKPGGNGGNSGNHSGS
SGGGQSSATAMAFGLPALATPGAEGLALSVSGDALSAAVADVLAALKGPFKFGLWGIAIYGV
LPSEIAKDDPKMMSKIVTSLPADTVTETPVSSLPLDQATVSVTKRVADIVKDERQHIAVVTG
RPMSVPVVDAKPTKRPGVFSVSIPGLPSLQVSVPKGVPAAKAPPKGIIAEKGDSRPAGFTAG
GNSREAVIRFPKESGQKPVYVSVTDVLTPAQVKQRLEEEKRRQQAWDAAHPEEGLKREYDKA
KAELDAEDKNIATLNSRIASTEKAIPGARAAVQEADKKVKEAEANKDDFVTYNPPHEYGSGW
QDQVRYLDKDIQNQNEKLKAAQTSLNEMNESLSRDKAALSGAMESRKQKEKKAKDAENKLNE
EKKKPRKGTKDYGHDYFPDPKTEDIKGLGELKEGKPKTPKQGGGGKRARWYGDKKRKIYEWD
SQHGELEGYRASDGEHLGAFDPKTGKQVKGPDPKRNIKKYL
EVSMGLKLNLTWFDKKTEEFKGGEYSKDFGDDGSVIESLGMPLKDNINNGWEDVEKSWVSIL
QPHFKNVIDISKFDYFVSFDYRDGNW
P806 Domain boundaries:
    1-296: 628 TD
297-436: 628 RD
437-537: 628 KD
538-625: 628 Immunity
Number of amino acids: 537
Molecular weight: 57145
Theoretical pI: 9.21
DNA sequence of P806 (SEQ ID NO: 32):
ATGAACGGTACATCAGGTAAAGGTGGCCCTTCATCAGGTGGTGGTACGGATCCAAACAGCGG
GCCGGGCTGGGGTACGACGCATACACCTAACGGGGATATTCATAACTATAACCCGGGGGAGT
TTGGTAACGGCGGGAGTAAACCCGGTGGTAATGGCGGTAACAGCGGCAATCATAGCGGGAGC
TCTGGTGGCGGACAGTCTTCGGCCACCGCGATGGCCTTCGGTCTGCCTGCCTTGGCCACTCC
GGGGGCTGAAGGACTGGCTTTATCCGTTTCCGGCGATGCGTTGTCGGCCGCTGTTGCTGATG
TGCTGGCTGCCCTGAAAGGTCCGTTTAAGTTTGGTCTGTGGGGGATTGCGATTTACGGAGTG
CTGCCTTCTGAGATAGCAAAAGATGATCCGAAAATGATGTCAAAAATCGTGACGTCATTGCC
GGCAGACACGGTAACGGAGACGCCGGTCAGCTCCCTGCCGCTGGACCAGGCGACGGTCAGCG
TCACTAAACGTGTGGCGGATATTGTGAAGGACGAGCGGCAGCATATTGCGGTTGTCACCGGC
CGGCCAATGAGTGTTCCTGTGGTGGATGCGAAACCGACAAAACGTCCGGGGGTATTCAGTGT
GTCGATTCCGGGTCTCCCGTCTCTGCAGGTGAGCGTACCTAAAGGTGTTCCGGCCGCGAAAG
CCCCGCCAAAAGGCATTATTGCTGAAAAAGGTGATTCACGTCCGGCTGGTTTTACGGCCGGT
GGTAACTCCCGTGAAGCCGTTATTCGTTTCCCGAAAGAGAGCGGACAGAAACCGGTCTATGT
GTCGGTGACGGATGTTCTTACTCCGGCACAGGTAAAACAGCGTCTGGAGGAAGAAAAGCGTC
GCCAGCAGGCATGGGACGCTGCTCACCCGGAAGAGGGGCTGAAAAGAGAGTATGATAAAGCG
AAAGCTGAGCTGGATGCTGAAGATAAAAATATTGCGACCTTAAACAGTCGCATTGCATCGAC
AGAGAAGGCGATCCCCGGTGCAAGGGCTGCTGTTCAGGAAGCCGATAAAAAGGTAAAAGAGG
CAGAGGCAAATAAAGATGATTTTGTGACTTATAACCCGCCTCATGAATATGGCTCCGGGTGG
CAGGATCAGGTTCGCTATCTTGATAAGGATATTCAGAATCAGAATGAGAAATTAAAAGCTGC
CCAGACATCTTTAAACGAAATGAATGAATCCTTATCCAGGGATAAGGCTGCGCTTTCCGGGG
CGATGGAGAGCCGGAAACAAAAGGAGAAAAAAGCGAAGGATGCAGAAAATAAATTAAATGAG
GAAAAGAAAAAACCTCGTAAGGGAACTAAAGATTACGGCCATGATTATTTTCCTGATCCCAA
GACTGAAGATATTAAGGGGTTGGGAGAGTTGAAAGAGGGTAAACCTAAAACCCCTAAACAAG
GTGGTGGTGGTAAGCGGGCCCGATGGTATGGTGATAAAAAGCGTAAAATTTATGAATGGGAT
TCCCAGCACGGTGAGCTTGAAGGGTACCGCGCCAGTGATGGCGAACACCTCGGGGCATTCGA
TCCAAAAACGGGTAAGCAGGTTAAAGGGCCGGATCCAAAACGAAACATTA
AAAAATATCTTTAA
GAGGTAAGTATGGGACTTAAATTAAATTTAACCTGGTTTGATAAGAAAACCGAAGAGTTTAA
AGGTGGTGAATACTCAAAAGACTTCGGTGATGATGGTTCTGTCATTGAAAGTCTGGGGATGC
CTTTAAAGGATAATATTAATAATGGTTGGTTTGATGTTGAAAAATCATGGGTTTCGATATTA
CAGCCACACTTTAAAAATGTAATCGATATTAGTAAATTTGATTACTTTGTATCATTTGATTA
TCGGGATGGTAACTGGTAA
17. P810 (Dual receptor binding domains: 764 TD RD-P774 RD-P764 KD):
Amino acid sequence of P810 (SEQ ID NO: 33):
MPEETLTVVGGGNNSCNVSWGGGNGNNGGAGYSGKYGGTSYEGATSMLKLNDRVLIQLYLCN
PLNPDYIGAPWGSDKDAESIIRANRDKAGKFKANIQNWKTSGTGSLGSPVVGKSYSSGDVDT
YSVSFGKEKYNVLYNRKKDSFTTAYVDGGANKPEHSQGQIFLQVTLPYYQSAKFCQDSMAWL
AQYVKTHGATDPLTIQVVANNIRYFLNADTNLCHNPRQNVWEAFHSEMTGSGPAPAKYDYKS
MSLKQMKDQAIAVVKLYLLNESQASVIDTTSGIITDSGKTLSGKLGDKYNTLAKEAADNIKN
FQGKKLRSFNDAMASINELANNPKMKLSQADKTVVSNALKQMDLSALADRFKGLEKAFTWGD
RLLKAEKIREGVVTGITTGDWQKLAFEVEAMYLSGVAGAVALGITTAMISTVAVALSLPAVA
VSALTVVSVIGISILTSYIDADKAKALNNAVLGLFK
P810 Domain boundaries:
      1-38: 764 TD
  39-160: 764 RD
161-253: 774 RD
254-470: 764 KD
Number of amino acids: 470
Molecular weight: 50452
Theoretical pI: 8.89
DNA sequence of P810 (SEQ ID NO: 34):
ATGCCTGAAGAAACATTGACTGTCGTAGGTGGTGGTAATAACAGCTGTAATGTTAGCTGGGG
TGGTGGCAATGGTAACAACGGTGGTGCTGGCTATTCTGGTAAATACGGTGGTACTAGCTATG
AAGGTGCAACTAGTATGTTGAAGTTGAATGACCGTGTTTTAATTCAGCTTTATCTTTGCAAT
CCGCTTAACCCAGATTATATTGGAGCACCTTGGGGCAGTGATAAAGATGCAGAATCAATTAT
CAGAGCTAACAGAGATAAAGCAGGAAAGTTCAAGGCTAATATTCAGAACTGGAAAACAAGCG
GTACAGGTTCTTTAGGTAGCCCTGTAGTTGGGAAATCTTATAGCTCAGGTGATGTAGATACT
TATTCGGTTAGCTTCGGAAAAGAAAAATACAATGTCCTGTATAACCGTAAAAAAGACTCTTT
TACTACAGCTTATGTCGATGGCGGCGCAAATAAACCTGAGCATAGCCAGGGTCAAATCTTCC
TGCAAGTGACCCTGCCGTACTATCAGAGCGCGAAGTTTTGCCAAGACAGCATGGCGTGGCTG
GCGCAGTACGTTAAAACCCACGGCGCGACCGATCCGCTGACCATCCAAGTGGTTGCGAACAA
CATTCGTTACTTCCTGAACGCGGACACCAACCTGTGCCACAACCCGCGTCAGAACGTGTGGG
AGGCGTTTCACAGCGAAATGACCGGTAGCGGTCCGGCGCCGGCGAAGTACGATTATAAAAGC
ATGAGCCTGAAGCAAATGAAGGATCAGGCTATTGCCGTTGTTAAACTTTACCTTCTCAACGA
AAGTCAGGCATCGGTAATCGATACTACATCGGGAATTATTACTGACTCTGGTAAAACTCTTA
GCGGGAAATTAGGTGATAAATACAACACTCTGGCGAAAGAAGCTGCTGACAATATAAAAAAC
TTCCAGGGTAAGAAACTCCGCAGTTTTAATGATGCTATGGCATCTATTAATGAACTAGCTAA
CAATCCAAAGATGAAGTTAAGTCAGGCGGATAAAACAGTCGTTTCTAATGCCCTCAAACAAA
TGGATTTGTCAGCACTAGCTGACCGATTCAAAGGGTTAGAGAAAGCCTTTACTTGGGGTGAT
CGACTTCTTAAAGCCGAGAAAATCAGAGAAGGTGTTGTTACTGGTATTACCACAGGGGACTG
GCAAAAGCTGGCGTTTGAGGTTGAAGCTATGTACCTCAGTGGTGTTGCTGGCGCCGTAGCGT
TAGGGATTACTACTGCCATGATTAGCACAGTCGCAGTCGCTTTGTCACTTCCAGCTGTAGCT
GTCTCTGCGCTTACTGTTGTGTCCGTCATTGGCATCTCTATTCTCACATCTTATATCGATGC
TGATAAGGCCAAAGCACTGAATAATGCAGTGCTTGGCTTATTTAAATAA
18. P812 (P628 TD RD-Rigid Linker-P764 KD):
Amino acid sequence of P812 (SEQ ID NO: 35):
MSGGDGRGPGNSGLGHNGGQASGNVNGTSGKGGPSSGGGTDPNSGPGWGTTHTPNGDIHNYN
PGEFGNGGSKPGGNGGNSGNHSGSSGGGQSSATAMAFGLPALATPGAEGLALSVSGDALSAA
VADVLAALKGPFKFGLWGIAIYGVLPSEIAKDDPKMMSKIVTSLPADTVTETPVSSLPLDQA
TVSVTKRVADIVKDERQHIAVVTGRPMSVPVVDAKPTKRPGVFSVSIPGLPSLQVSVPKGVP
AAKAPPKGIIAEKGDSRPAGFTAGGNSREAVIRFPKESGQKPVYVSVTDVLTPAQVKQRLEE
EKRRQQAWDAAHPEEGLKREYDKAKAELDAEDKNIATLNSRIASTEKAIPGARAAVQEADKK
VKEAEANKDDFVTYNPPHEYGSGWQDQVRYLDKDIQNQNEKLKAAQTSLNEMNESLSRDKAA
LSGAMESRKQKEKKAKDAENKLNEEKEAAAKEAAAKMKDQAIAVVKLYLLNESQASVIDTTS
GIITDSGKTLSGKLGDKYNTLAKEAADNIKNFQGKKLRSFNDAMASINELANNPKMKLSQAD
KTVVSNALKQMDLSALADRFKGLEKAFTWGDRLLKAEKIREGVVTGITTGDWQKLAFEVEAM
YLSGVAGAVALGITTAMISTVAVALSLPAVAVSALTVVSVIGISILTSYIDADKAKALNNAV
LGLFK
P812 Domain boundaries:
    1-320: 628 TD
321-460: 628 RD
461-470: rigid linker
471-687: 764 KD
Number of amino acids: 687
Molecular weight: 71637
Theoretical pI: 8.77
DNA sequence of P812 (SEQ ID NO: 36):
ATGAGTGGTGGAGACGGACGAGGTCCGGGTAATTCAGGTCTGGGACACAATGGTGGTCAGGC
CAGTGGGAATGTGAACGGTACATCAGGTAAAGGTGGCCCTTCATCAGGTGGTGGTACGGATC
CAAACAGCGGGCCGGGCTGGGGTACGACGCATACACCTAACGGGGATATTCATAACTATAAC
CCGGGGGAGTTTGGTAACGGCGGGAGTAAACCCGGTGGTAATGGCGGTAACAGCGGCAATCA
TAGCGGGAGCTCTGGTGGCGGACAGTCTTCGGCCACCGCGATGGCCTTCGGTCTGCCTGCCT
TGGCCACTCCGGGGGCTGAAGGACTGGCTTTATCCGTTTCCGGCGATGCGTTGTCGGCCGCT
GTTGCTGATGTGCTGGCTGCCCTGAAAGGTCCGTTTAAGTTTGGTCTGTGGGGGATTGCGAT
TTACGGAGTGCTGCCTTCTGAGATAGCAAAAGATGATCCGAAAATGATGTCAAAAATCGTGA
CGTCATTGCCGGCAGACACGGTAACGGAGACGCCGGTCAGCTCCCTGCCGCTGGACCAGGCG
ACGGTCAGCGTCACTAAACGTGTGGCGGATATTGTGAAGGACGAGCGGCAGCATATTGCGGT
TGTCACCGGCCGGCCAATGAGTGTTCCTGTGGTGGATGCGAAACCGACAAAACGTCCGGGGG
TATTCAGTGTGTCGATTCCGGGTCTCCCGTCTCTGCAGGTGAGCGTACCTAAAGGTGTTCCG
GCCGCGAAAGCCCCGCCAAAAGGCATTATTGCTGAAAAAGGTGATTCACGTCCGGCTGGTTT
TACGGCCGGTGGTAACTCCCGTGAAGCCGTTATTCGTTTCCCGAAAGAGAGCGGACAGAAAC
CGGTCTATGTGTCGGTGACGGATGTTCTTACTCCGGCACAGGTAAAACAGCGTCTGGAGGAA
GAAAAGCGTCGCCAGCAGGCATGGGACGCTGCTCACCCGGAAGAGGGGCTGAAAAGAGAGTA
TGATAAAGCGAAAGCTGAGCTGGATGCTGAAGATAAAAATATTGCGACCTTAAACAGTCGCA
TTGCATCGACAGAGAAGGCGATCCCCGGTGCAAGGGCTGCTGTTCAGGAAGCCGATAAAAAG
GTAAAAGAGGCAGAGGCAAATAAAGATGATTTTGTGACTTATAACCCGCCTCATGAATATGG
CTCCGGGTGGCAGGATCAGGTTCGCTATCTTGATAAGGATATTCAGAATCAGAATGAGAAAT
TAAAAGCTGCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGCAGAAAATAA
ATTAAATGAGGAAAAGGAAGCCGCTGCCAAGGAGGCGGCCGCTAAAATGAAGGATCAGGCTATTGCC
GTTGTTAAACTTTACCTTCTCAACGAAAGTCAGGCATCGGTAATCGATACTACATCGGGAATTATTACTGACTCTGGTA
AAACTCTTAGCGGGAAATTAGGTGATAAATACAACACTCTGGCGAAAGAAGCTGCTGACAATATAAAAAACTTCCAGGG
TAAGAAACTCCGCAGTTTTAATGATGCTATGGCATCTATTAATGAACTAGCTAACAATCCAAAGATGAAGTTAAGTCAG
GCGGATAAAACAGTCGTTTCTAATGCCCTCAAACAAATGGATTTGTCAGCACTAGCTGACCGATTCAAAGGGTTAGAGA
AAGCCTTTACTTGGGGTGATCGACTTCTTAAAGCCGAGAAAATCAGAGAAGGTGTTGTTACTGGTATTACCACAGGGGA
CTGGCAAAAGCTGGCGTTTGAGGTTGAAGCTATGTACCTCAGTGGTGTTGCTGGCGCCGTAGCGTTAGGGATTACTACT
GCCATGATTAGCACAGTCGCAGTCGCTTTGTCACTTCCAGCTGTAGCTGTCTCTGCGCTTACTGTTGTGTCCGTCATTG
GCATCTCTATTCTCACATCTTATATCGATGCTGATAAGGCCAAAGCACTGAATAATGCAGTGCTTGGCTTATTTAAATA
A
19. P818 (P764 TD-P774 RD-P764 KD):
Amino acid sequence of P818 (SEQ ID NO: 37):
MPEETLTVVGGGNNSCNVSWGGGNGNNGGAGYSGKYGGTPSQGQIFLQVTLPYYQSAKFCQD
SMAWLAQYVKTHGATDPLTIQVVANNIRYFLNADTNLCHNPRQNVWEAFHSEMTGSGPAPAK
YDYKSMSLKLQAIAVVKLYLLNESQASVIDTTSGIITDSGKTLSGKLGDKYNTLAKEAADNI
KNFQGKKLRSENDAMASINELANNPKMKLSQADKTVVSNALKQMDLSALADRFKGLEKAFTW
GDRLLKAEKIREGVVTGITTGDWQKLAFEVEAMYLSGVAGAVALGITTAMISTVAVALSLPA
VAVSALTVVSVIGISILTSYIDADKAKALNNAVLGLFK
P818 Domain boundaries:
        1-37: 764 TD
    38, 39: Kpn I restriction site
    40-132: 774 RD
133, 134: Hind III restriction site
  135-348: 764 KD
Number of amino acids: 348
Molecular weight: 37028
Theoretical pI: 8.74
DNA sequence of P818 (SEQ ID NO: 38):
ATGCCTGAAGAAACATTGACTGTCGTAGGTGGTGGTAATAACAGCTGTAATGTTAGCTGGGG
TGGTGGCAATGGTAACAACGGTGGTGCTGGCTATTCTGGTAAATACGGTGGTACCCCGAGCC
AGGGTCAAATCTTCCTGCAAGTGACCCTGCCGTACTATCAGAGCGCGAAGTTTTGCCAAGAC
AGCATGGCGTGGCTGGCGCAGTACGTTAAAACCCACGGCGCGACCGATCCGCTGACCATCCA
AGTGGTTGCGAACAACATTCGTTACTTCCTGAACGCGGACACCAACCTGTGCCACAACCCGC
GTCAGAACGTGTGGGAGGCGTTTCACAGCGAAATGACCGGTAGCGGTCCGGCGCCGGCGAAG
TACGATTATAAAAGCATGAGCCTGAAGCTTCAGGCTATTGCCGTTGTTAAACTTTACCTTCT
CAACGAAAGTCAGGCATCGGTAATCGATACTACATCGGGAATTATTACTGACTCTGGTAAAA
CTCTTAGCGGGAAATTAGGTGATAAATACAACACTCTGGCGAAAGAAGCTGCTGACAATATA
AAAAACTTCCAGGGTAAGAAACTCCGCAGTTTTAATGATGCTATGGCATCTATTAATGAACT
AGCTAACAATCCAAAGATGAAGTTAAGTCAGGCGGATAAAACAGTCGTTTCTAATGCCCTCA
AACAAATGGATTTGTCAGCACTAGCTGACCGATTCAAAGGGTTAGAGAAAGCCTTTACTTGG
GGTGATCGACTTCTTAAAGCCGAGAAAATCAGAGAAGGTGTTGTTACTGGTATTACCACAGG
GGACTGGCAAAAGCTGGCGTTTGAGGTTGAAGCTATGTACCTCAGTGGTGTTGCTGGCGCCG
TAGCGTTAGGGATTACTACTGCCATGATTAGCACAGTCGCAGTCGCTTTGTCACTTCCAGCT
GTAGCTGTCTCTGCGCTTACTGTTGTGTCCGTCATTGGCATCTCTATTCTCACATCTTATAT
CGATGCTGATAAGGCCAAAGCACTGAATAATGCAGTGCTTGGCTTATTTAAATAA
20. P819 (P774 TD-P764 RD-P774 KD):
Amino acid sequence of P819 (SEQ ID NO: 39):
MSETMVVVAPPTGFEPAGYGGGLESPSTPNGTSYEGATSMLKLNDRVLIQLYLCNPLNPDYI
GAPWGSDKDAESIIRANRDKAGKFKANIQNWKTSGTGSLGSPVVGKSYSSGDVDTYSVSFGK
EKYNVLYNRKKDSFTTAYVDGGANKPEHSMKLMSGNVVTPAAAFGHYLWGNGEERYVNLPDV
GLKITPQQIPELMNIVNSGVTGNIPVDLNFNHNTYDSGGVIPASYLGNVSLRTIGSLNIQNS
GEWTYNGVVRAYNDYYDFNLGDYRGAIAESLTYLGAQFSGKPYHIAMPGEINISGAGHR
P819 Domain boundaries:
        1-30: 774 TD
    31, 32: Kpn I restriction site
    33-154: 764 RD
155, 156: Hind III restriction site
  157-307: 774 KD
Number of amino acids: 307
Molecular weight: 33073
Theoretical pI: 5.96
DNA sequence of P819 (SEQ ID NO: 40):
ATGAGCGAGACCATGGTTGTTGTTGCGCCGCCGACCGGTTTTGAGCCGGCGGGTTACGGTGG
TGGCCTGTTCAGCCCGAGCACCCCGAACGGTACCAGCTATGAAGGTGCAACTAGTATGTTGA
AGTTGAATGACCGTGTTTTAATTCAGCTTTATCTTTGCAATCCGCTTAACCCAGATTATATT
GGAGCACCTTGGGGCAGTGATAAAGATGCAGAATCAATTATCAGAGCTAACAGAGATAAAGC
AGGAAAGTTCAAGGCTAATATTCAGAACTGGAAAACAAGCGGTACAGGTTCTTTAGGTAGCC
CTGTAGTTGGGAAATCTTATAGCTCAGGTGATGTAGATACTTATTCGGTTAGCTTCGGAAAA
GAAAAATACAATGTCCTGTATAACCGTAAAAAAGACTCTTTTACTACAGCTTATGTCGATGG
CGGCGCAAATAAACCTGAGCATAGCATGAAGCTTATGAGCGGTAACGTGGTTACCCCGGCGG
CGGCGTTTGGTCACTACCTGTGGGGTAACGGCGAGGAACGTTATGTGAACCTGCCGGACGTT
GGTCTGAAAATCACCCCGCAGCAAATTCCGGAGCTGATGAACATCGTGAACAGCGGTGTTAC
CGGCAACATTCCGGTTGACCTGAACTTTAACCACAACACCTACGATAGCGGTGGCGTGATCC
CGGCGAGCTATCTGGGTAACGTTAGCCTGCGTACCATCGGCAGCCTGAACATTCAGAACAGC
GGCGAGTGGACCTACAACGGCGTGGTTCGTGCGTATAACGACTACTATGATTTCAACCTGGG
TGATTACCGTGGCGCGATTGCGGAAAGCCTGACCTATCTGGGCGCGCAATTTAGCGGTAAGC
CGTATCACATTGCGATGCCGGGCGAAATCAACATTAGCGGTGCGGGTCATCGTTAA
21. P820 (P764 TD RD-Rigid Linker-P628 Δ25):
Amino acid sequence of P820 (SEQ ID NO: 41):
MPEETLTVVGGGNNSCNVSWGGGNGNNGGAGYSGKYGGTSYEGATSMLKLNDRVLIQLYLCN
PLNPDYIGAPWGSDKDAESIIRANRDKAGKFKANIQNWKTSGTGSLGSPVVGKSYSSGDVDT
YSVSFGKEKYNVLYNRKKDSFTTAYVDGGANKPEHEAAAKEAAAKNGTSGKGGPSSGGGTDP
NSGPGWGTTHTPNGDIHNYNPGEFGNGGSKPGGNGGNSGNHSGSSGGGQSSATAMAFGLPAL
ATPGAEGLALSVSGDALSAAVADVLAALKGPFKFGLWGIAIYGVLPSEIAKDDPKMMSKIVT
SLPADTVTETPVSSLPLDQATVSVTKRVADIVKDERQHIAVVTGRPMSVPVVDAKPTKRPGV
FSVSIPGLPSLQVSVPKGVPAAKAPPKGIIAEKGDSRPAGFTAGGNSREAVIRFPKESGQKP
VYVSVTDVLTPAQVKQRLEEEKRRQQAWDA

[symbol]

[symbol]

[symbol]

[symbol]

[symbol]

[symbol]

[symbol]

[symbol]

[symbol]

KKPRKGTKDYGHDYFP
DPKTEDIKGLGELKEGKPKTPKQGGGGKRARWYGDKKRKIYEWDSQHGELEGYRASDGEHLG
AFDPKTGKQVKGPDPKRNIKKYL
EVSMGLKLNLTWFDKKTEEFKGGEYSKDFGDDGSVIESLGMPLKDNINNGWEDVEKSWVSIL
QPHFKNVIDISKFDYFVSFDYRDGNW
P820 domain boundaries:
1-38: 764 TD
  39-159: P764 RD
160-169: Rigid linker
170-464: P628 TD

[symbol]

605-705: P628 KD
706-793: P628 Immunity
Number of amino acids: 705
Molecular weight: 74790
Theoretical pI: 9.10
DNA sequence of P820 (SEQ ID NO: 42):
ATGCCTGAAGAAACATTGACTGTCGTAGGTGGTGGTAATAACAGCTGTAATGTTAGCTGGGG
TGGTGGCAATGGTAACAACGGTGGTGCTGGCTATTCTGGTAAATACGGTGGTACTAGCTATG
AAGGTGCAACTAGTATGTTGAAGTTGAATGACCGTGTTTTAATTCAGCTTTATCTTTGCAAT
CCGCTTAACCCAGATTATATTGGAGCACCTTGGGGCAGTGATAAAGATGCAGAATCAATTAT
CAGAGCTAACAGAGATAAAGCAGGAAAGTTCAAGGCTAATATTCAGAACTGGAAAACAAGCG
GTACAGGTTCTTTAGGTAGCCCTGTAGTTGGGAAATCTTATAGCTCAGGTGATGTAGATACT
TATTCGGTTAGCTTCGGAAAAGAAAAATACAATGTCCTGTATAACCGTAAAAAAGACTCTTT
TACTACAGCTTATGTCGATGGCGGCGCAAATAAACCTGAGCATGAAGCCGCTGCCAAGGAGG
CGGCCGCTAAAAACGGTACATCAGGTAAAGGTGGCCCTTCATCAGGTGGTGGTACGGATCCA
AACAGCGGGCCGGGCTGGGGTACGACGCATACACCTAACGGGGATATTCATAACTATAACCC
GGGGGAGTTTGGTAACGGCGGGAGTAAACCCGGTGGTAATGGCGGTAACAGCGGCAATCATA
GCGGGAGCTCTGGTGGCGGACAGTCTTCGGCCACCGCGATGGCCTTCGGTCTGCCTGCCTTG
GCCACTCCGGGGGCTGAAGGACTGGCTTTATCCGTTTCCGGCGATGCGTTGTCGGCCGCTGT
TGCTGATGTGCTGGCTGCCCTGAAAGGTCCGTTTAAGTTTGGTCTGTGGGGGATTGCGATTT
ACGGAGTGCTGCCTTCTGAGATAGCAAAAGATGATCCGAAAATGATGTCAAAAATCGTGACG
TCATTGCCGGCAGACACGGTAACGGAGACGCCGGTCAGCTCCCTGCCGCTGGACCAGGCGAC
GGTCAGCGTCACTAAACGTGTGGCGGATATTGTGAAGGACGAGCGGCAGCATATTGCGGTTG
TCACCGGCCGGCCAATGAGTGTTCCTGTGGTGGATGCGAAACCGACAAAACGTCCGGGGGTA
TTCAGTGTGTCGATTCCGGGTCTCCCGTCTCTGCAGGTGAGCGTACCTAAAGGTGTTCCGGC
CGCGAAAGCCCCGCCAAAAGGCATTATTGCTGAAAAAGGTGATTCACGTCCGGCTGGTTTTA
CGGCCGGTGGTAACTCCCGTGAAGCCGTTATTCGTTTCCCGAAAGAGAGCGGACAGAAACCG
GTCTATGTGTCGGTGACGGATGTTCTTACTCCGGCACAGGTAAAACAGCGTCTGGAGGAAGA
AAAGCGTCGCCAGCAGGCATGGGACGCTGCTCACCCGGAAGAGGGGCTGAAAAGAGAGTATG
ATAAAGCGAAAGCTGAGCTGGATGCTGAAGATAAAAATATTGCGACCTTAAACAGTCGCATT
GCATCGACAGAGAAGGCGATCCCCGGTGCAAGGGCTGCTGTTCAGGAAGCCGATAAAAAGGT
AAAAGAGGCAGAGGCAAATAAAGATGATTTTGTGACTTATAACCCGCCTCATGAATATGGCT
CCGGGTGGCAGGATCAGGTTCGCTATCTTGATAAGGATATTCAGAATCAGAATGAGAAATTA
AAAGCTGCCCAGACATCTTTAAACGAAATGAATGAATCCTTATCCAGGGATAAGGCTGCGCT
TTCCGGGGCGATGGAGAGCCGGAAACAAAAGGAGAAAAAAGCGAAGGATGCAGAAAATAAAT
TAAATGAGGAAAAGAAAAAACCTCGTAAGGGAACTAAAGATTACGGCCATGATTATTTTCCT
GATCCCAAGACTGAAGATATTAAGGGGTTGGGAGAGTTGAAAGAGGGTAAACCTAAAACCCC
TAAACAAGGTGGTGGTGGTAAGCGGGCCCGATGGTATGGTGATAAAAAGCGTAAAATTTATG
AATGGGATTCCCAGCACGGTGAGCTTGAAGGGTACCGCGCCAGTGATGGCGAACACCTCGGG
GCATTCGATCCAAAAACGGGTAAGCAGGTTAAAGGGCCGGATCCAAAACGAAACATTAAAAA
ATATCTTTAA
GAGGTAAGTATGGGACTTAAATTAAATTTAACCTGGTTTGATAAGAAAACCGAAGAGTTTAA
AGGTGGTGAATACTCAAAAGACTTCGGTGATGATGGTTCTGTCATTGAAAGTCTGGGGATGC
CTTTAAAGGATAATATTAATAATGGTTGGTTTGATGTTGAAAAATCATGGGTTTCGATATTA
CAGCCACACTTTAAAAATGTAATCGATATTAGTAAATTTGATTACTTTGTATCATTTGATTA
TCGGGATGGTAACTGGTAA
22. P822 (P801 TD RD-Rigid linker-P764 KD):
Amino acid sequence of P822 (SEQ ID NO: 43):
MSDTMIVVATPTPGFSYASGLTYGGGAFAGAPANGPSEGQIFFQTVLPAYQSPNLCIGQLAW
MTDYINKNGVGNPKTWEVISQNVLIFCSADTALVLNPRIAVYDGFHKTKWAPAKENFKTQSQ
EKFSGNVTTPIAAFGHYLWGEGKPRTVDLSSEAAAKEAAAKMKDQAIAVVKLYLLNESQASV
IDTTSGIITDSGKTLSGKLGDKYNTLAKEAADNIKNFQGKKLRSFNDAMASINELANNPKMK
LSQADKTVVSNALKQMDLSALADRFKGLEKAFTWGDRLLKAEKIREGVVTGITTGDWQKLAF
EVEAMYLSGVAGAVALGITTAMISTVAVALSLPAVAVSALTVVSVIGISILTSYIDADKAKA
LNNAVLGLFK
P822 Domain boundaries:
      1-39: 801 TD
  40-155: 801 RD
156-165: rigid linker
166-382: 764 TD
Number of amino acids: 382
Molecular weight: 40558
Theoretical pI: 8.91
DNA sequence of P822 (SEQ ID NO: 44):
ATGTCAGATACAATGATAGTAGTTGCTACTCCGACCCCGGGTTTTAGCTACGCGAGCGGCTT
GACGTACGGCGGTGGTGCGTTTGCGGGTGCGCCTGCGAACGGTCCGTCGGAAGGCCAAATCT
TTTTCCAGACCGTTCTGCCGGCGTATCAATCCCCGAATCTGTGTATTGGTCAACTGGCTTGG
ATGACCGATTACATTAATAAGAACGGGGTGGGTAACCCAAAAACCTGGGAAGTTATTTCCCA
AAACGTTCTGATTTTCTGCAGCGCGGATACTGCCCTGGTGCTTAATCCGCGCATCGCGGTGT
ACGACGGCTTCCACAAAACTAAATGGGCGCCAGCCAAGTTCAATTTTAAAACCCAGAGCCAG
GAGAAATTCTCTGGCAACGTGACCACCCCGATTGCTGCCTTCGGCCATTATCTGTGGGGTGA
GGGCAAGCCGCGTACCGTCGACTTGTCTAGCGAAGCCGCTGCCAAGGAGGCGGCCGCTAAAA
TGAAGGATCAGGCTATTGCCGTTGTTAAACTTTACCTTCTCAACGAAAGTCAGGCATCGGTA
ATCGATACTACATCGGGAATTATTACTGACTCTGGTAAAACTCTTAGCGGGAAATTAGGTGA
TAAATACAACACTCTGGCGAAAGAAGCTGCTGACAATATAAAAAACTTCCAGGGTAAGAAAC
TCCGCAGTTTTAATGATGCTATGGCATCTATTAATGAACTAGCTAACAATCCAAAGATGAAG
TTAAGTCAGGCGGATAAAACAGTCGTTTCTAATGCCCTCAAACAAATGGATTTGTCAGCACT
AGCTGACCGATTCAAAGGGTTAGAGAAAGCCTTTACTTGGGGTGATCGACTTCTTAAAGCCG
AGAAAATCAGAGAAGGTGTTGTTACTGGTATTACCACAGGGGACTGGCAAAAGCTGGCGTTT
GAGGTTGAAGCTATGTACCTCAGTGGTGTTGCTGGCGCCGTAGCGTTAGGGATTACTACTGC
CATGATTAGCACAGTCGCAGTCGCTTTGTCACTTCCAGCTGTAGCTGTCTCTGCGCTTACTG
TTGTGTCCGTCATTGGCATCTCTATTCTCACATCTTATATCGATGCTGATAAGGCCAAAGCA
CTGAATAATGCAGTGCTTGGCTTATTTAAATAA
23. P821 (P628 TD and RD fusion to P801 full-length with rigid linker):
Amino acid sequence of P821 (SEQ ID NO: 45):
MSGGDGRGPGNSGLGHNGGQASGNVNGTSGKGGPSSGGGTDPNSGPGWGTTHTPNGDIHNYN
PGEFGNGGSKPGGNGGNSGNHSGSSGGGQSSATAMAFGLPALATPGAEGLALSVSGDALSAA
VADVLAALKGPFKFGLWGIAIYGVLPSEIAKDDPKMMSKIVTSLPADTVTETPVSSLPLDQA
TVSVTKRVADIVKDERQHIAVVTGRPMSVPVVDAKPTKRPGVFSVSIPGLPSLQVSVPKGVP
AAKAPPKGIIAEKGDSRPAGFTAGGNSREAVIRFPKESGQKPVYVSVTDVLTPAQVKQRLEE
EKRRQQAWDAAHPEEGLKREYDKAKAELDAEDKNIATLNSRIASTEKAIPGARAAVQEADKK
VKEAEANKDDFVTYNPPHEYGSGWQDQVRYLDKDIQNQNEKLKAAQTSLNEMNESLSRDKAA
LSGAMESRKQKEKKAKDAENKLNEEKEAAAKEAAAKSDTMIVVATPTPGFSYASGLTYGGGA
FAGAPANGPSEGQIFFQTVLPAYQSPNLCIGQLAWMTDYINKNGVGNPKTWEVISQNVLIFC
SADTALVLNPRIAVYDGFHKTKWAPAKFNFKTQSQEKFSGNVTTPIAAFGHYLWGEGKPRTV
DLSSVGLKIQANQIDPVMIAVKNNAAGTYQISGNFNRNTFIDGDIPGLYLGNITMKTEGTLK
IDAKGNWNYNGVVRAFNDTYDANPSTHRSKSAEDLTTLLRLTQGTPYEIRIPGELKVSGSGK
K
P821 Domain boundaries:
    1-320: 628 TD
320-460: 628 RD
461-470: rigid linker

[symbol]

509-612: 801 RD
613-745: 801 KD
Number of amino acids: 745
Molecular weight: 78379
Theoretical pI: 8.67
DNA sequence of P821 (SEQ ID NO: 46):
ATGAGTGGTGGAGACGGACGAGGTCCGGGTAATTCAGGTCTGGGACACAATGGTGGTCAGGC
CAGTGGGAATGTGAACGGTACATCAGGTAAAGGTGGCCCTTCATCAGGTGGTGGTACGGATC
CAAACAGCGGGCCGGGCTGGGGTACGACGCATACACCTAACGGGGATATTCATAACTATAAC
CCGGGGGAGTTTGGTAACGGCGGGAGTAAACCCGGTGGTAATGGCGGTAACAGCGGCAATCA
TAGCGGGAGCTCTGGTGGCGGACAGTCTTCGGCCACCGCGATGGCCTTCGGTCTGCCTGCCT
TGGCCACTCCGGGGGCTGAAGGACTGGCTTTATCCGTTTCCGGCGATGCGTTGTCGGCCGCT
GTTGCTGATGTGCTGGCTGCCCTGAAAGGTCCGTTTAAGTTTGGTCTGTGGGGGATTGCGAT
TTACGGAGTGCTGCCTTCTGAGATAGCAAAAGATGATCCGAAAATGATGTCAAAAATCGTGA
CGTCATTGCCGGCAGACACGGTAACGGAGACGCCGGTCAGCTCCCTGCCGCTGGACCAGGCG
ACGGTCAGCGTCACTAAACGTGTGGCGGATATTGTGAAGGACGAGCGGCAGCATATTGCGGT
TGTCACCGGCCGGCCAATGAGTGTTCCTGTGGTGGATGCGAAACCGACAAAACGTCCGGGGG
TATTCAGTGTGTCGATTCCGGGTCTCCCGTCTCTGCAGGTGAGCGTACCTAAAGGTGTTCCG
GCCGCGAAAGCCCCGCCAAAAGGCATTATTGCTGAAAAAGGTGATTCACGTCCGGCTGGTTT
TACGGCCGGTGGTAACTCCCGTGAAGCCGTTATTCGTTTCCCGAAAGAGAGCGGACAGAAAC
CGGTCTATGTGTCGGTGACGGATGTTCTTACTCCGGCACAGGTAAAACAGCGTCTGGAGGAA
GAAAAGCGTCGCCAGCAGGCATGGGACGCTGCTCACCCGGAAGAGGGGCTGAAAAGAGAGTA
TGATAAAGCGAAAGCTGAGCTGGATGCTGAAGATAAAAATATTGCGACCTTAAACAGTCGCA
TTGCATCGACAGAGAAGGCGATCCCCGGTGCAAGGGCTGCTGTTCAGGAAGCCGATAAAAAG
GTAAAAGAGGCAGAGGCAAATAAAGATGATTTTGTGACTTATAACCCGCCTCATGAATATGG
CTCCGGGTGGCAGGATCAGGTTCGCTATCTTGATAAGGATATTCAGAATCAGAATGAGAAAT
TAAAAGCTGCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGCAGAAAATAA
ATTAAATGAGGAAAAGGAAGCCGCTGCCAAGGAGGCGGCCGCTAAATCAGATACAATGATAG
TAGTTGCTACTCCGACCCCGGGTTTTAGCTACGCGAGCGGCTTGACGTACGGCGGTGGTGCG
TTTGCGGGTGCGCCTGCGAACGGTCCGTCGGAAGGCCAAATCTTTTTCCAGACCGTTCTGCC
GGCGTATCAATCCCCGAATCTGTGTATTGGTCAACTGGCTTGGATGACCGATTACATTAATA
AGAACGGGGTGGGTAACCCAAAAACCTGGGAAGTTATTTCCCAAAACGTTCTGATTTTCTGC
AGCGCGGATACTGCCCTGGTGCTTAATCCGCGCATCGCGGTGTACGACGGCTTCCACAAAAC
TAAATGGGCGCCAGCCAAGTTCAATTTTAAAACCCAGAGCCAGGAGAAATTCTCTGGCAACG
TGACCACCCCGATTGCTGCCTTCGGCCATTATCTGTGGGGTGAGGGCAAGCCGCGTACCGTC
GACTTGTCTAGCGTTGGTCTGAAGATCCAGGCAAACCAGATCGACCCGGTTATGATTGCGGT
TAAAAACAACGCAGCAGGCACCTACCAGATCTCCGGCAACTTCAACCGTAATACCTTTATCG
ACGGCGATATTCCGGGTTTATATCTGGGTAACATTACCATGAAAACGGAAGGCACACTCAAG
ATCGACGCCAAGGGTAATTGGAATTATAACGGTGTCGTGCGCGCGTTTAACGATACCTACGA
CGCTAATCCGTCGACCCACCGTAGCAAAAGCGCAGAAGATCTGACGACGTTGCTGCGTTTGA
CGCAAGGTACGCCGTACGAGATCAGAATCCCGGGTGAGCTGAAGGTGAGCGGTAGCGGCAAG
AAGTAA
24. P823 (P628 TD and RD fusion to P801 full-length with flexible linker):
Amino acid sequence of P823 (SEQ ID NO: 47):
MSGGDGRGPGNSGLGHNGGQASGNVNGTSGKGGPSSGGGTDPNSGPGWGTTHTPNGDIHNYN
PGEFGNGGSKPGGNGGNSGNHSGSSGGGQSSATAMAFGLPALATPGAEGLALSVSGDALSAA
VADVLAALKGPFKFGLWGIAIYGVLPSEIAKDDPKMMSKIVTSLPADTVTETPVSSLPLDQA
TVSVTKRVADIVKDERQHIAVVTGRPMSVPVVDAKPTKRPGVESVSIPGLPSLQVSVPKGVP
AAKAPPKGIIAEKGDSRPAGFTAGGNSREAVIRFPKESGQKPVYVSVTDVLTPAQVKQRLEE
EKRRQQAWDAAHPEEGLKREYDKAKAELDAEDKNIATLNSRIASTEKAIPGARAAVQEADKK
VKEAEANKDDFVTYNPPHEYGSGWQDQVRYLDKDIQNQNEKLKAAQTSLNEMNESLSRDKAA
LSGAMESRKQKEKKAKDAENKLNEEKGSGSASGSDTMIVVATPTPGFSYASGLTYGGGAFAG
APANGPSEGQIFFQTVLPAYQSPNLCIGQLAWMTDYINKNGVGNPKTWEVISQNVLIFCSAD
TALVLNPRIAVYDGFHKTKWAPAKFNFKTQSQEKFSGNVTTPIAAFGHYLWGEGKPRTVDLS
SVGLKIQANQIDPVMIAVKNNAAGTYQISGNFNRNTFIDGDIPGLYLGNITMKTEGTLKIDA
KGNWNYNGVVRAFNDTYDANPSTHRSKSAEDLTTLLRLTQGTPYEIRIPGELKVSGSGKK
P823 Domain boundaries:
    1-320: 628 TD
321-460: 628 RD
461-467: flexible linker
468-505: 801 TD
506-609: 801 RD
610-742: 801 KD
Number of amino acids: 742
Molecular weight: 77942
Theoretical pI: 8.68
DNA sequence of P823 (SEQ ID NO: 48):
ATGAGTGGTGGAGACGGACGAGGTCCGGGTAATTCAGGTCTGGGACACAATGGT
GGTCAGGCCAGTGGGAATGTGAACGGTACATCAGGTAAAGGTGGCCCTTCATCA
GGTGGTGGTACGGATCCAAACAGCGGGCCGGGCTGGGGTACGACGCATACACCT
AACGGGGATATTCATAACTATAACCCGGGGGAGTTTGGTAACGGCGGGAGTAAA
CCCGGTGGTAATGGCGGTAACAGCGGCAATCATAGCGGGAGCTCTGGTGGCGGA
CAGTCTTCGGCCACCGCGATGGCCTTCGGTCTGCCTGCCTTGGCCACTCCGGGGG
CTGAAGGACTGGCTTTATCCGTTTCCGGCGATGCGTTGTCGGCCGCTGTTGCTGA
TGTGCTGGCTGCCCTGAAAGGTCCGTTTAAGTTTGGTCTGTGGGGGATTGCGATT
TACGGAGTGCTGCCTTCTGAGATAGCAAAAGATGATCCGAAAATGATGTCAAAA
ATCGTGACGTCATTGCCGGCAGACACGGTAACGGAGACGCCGGTCAGCTCCCTG
CCGCTGGACCAGGCGACGGTCAGCGTCACTAAACGTGTGGCGGATATTGTGAAG
GACGAGCGGCAGCATATTGCGGTTGTCACCGGCCGGCCAATGAGTGTTCCTGTGG
TGGATGCGAAACCGACAAAACGTCCGGGGGTATTCAGTGTGTCGATTCCGGGTCT
CCCGTCTCTGCAGGTGAGCGTACCTAAAGGTGTTCCGGCCGCGAAAGCCCCGCCA
AAAGGCATTATTGCTGAAAAAGGTGATTCACGTCCGGCTGGTTTTACGGCCGGTG
GTAACTCCCGTGAAGCCGTTATTCGTTTCCCGAAAGAGAGCGGACAGAAACCGG
TCTATGTGTCGGTGACGGATGTTCTTACTCCGGCACAGGTAAAACAGCGTCTGGA
GGAAGAAAAGCGTCGCCAGCAGGCATGGGACGCTGCTCACCCGGAAGAGGGGC
TGAAAAGAGAGTATGATAAAGCGAAAGCTGAGCTGGATGCTGAAGATAAAAAT
ATTGCGACCTTAAACAGTCGCATTGCATCGACAGAGAAGGCGATCCCCGGTGCA
AGGGCTGCTGTTCAGGAAGCCGATAAAAAGGTAAAAGAGGCAGAGGCAAATAA
AGATGATTTTGTGACTTATAACCCGCCTCATGAATATGGCTCCGGGTGGCAGGAT
CAGGTTCGCTATCTTGATAAGGATATTCAGAATCAGAATGAGAAATTAAAAGCT
GCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGC
AGAAAATAAATTAAATGAGGAAAAGGGCTCCGGATCGGCTTCTGGGTCAGATAC
AATGATAGTAGTTGCTACTCCGACCCCGGGTTTTAGCTACGCGAGCGGCTTGACG
TACGGCGGTGGTGCGTTTGCGGGTGCGCCTGCGAACGGTCCGTCGGAAGGCCAA
ATCTTTTTCCAGACCGTTCTGCCGGCGTATCAATCCCCGAATCTGTGTATTGGTCA
ACTGGCTTGGATGACCGATTACATTAATAAGAACGGGGTGGGTAACCCAAAAAC
CTGGGAAGTTATTTCCCAAAACGTTCTGATTTTCTGCAGCGCGGATACTGCCCTG
GTGCTTAATCCGCGCATCGCGGTGTACGACGGCTTCCACAAAACTAAATGGGCGC
CAGCCAAGTTCAATTTTAAAACCCAGAGCCAGGAGAAATTCTCTGGCAACGTGA
CCACCCCGATTGCTGCCTTCGGCCATTATCTGTGGGGTGAGGGCAAGCCGCGTAC
CGTCGACTTGTCTAGCGTTGGTCTGAAGATCCAGGCAAACCAGATCGACCCGGTT
ATGATTGCGGTTAAAAACAACGCAGCAGGCACCTACCAGATCTCCGGCAACTTC
AACCGTAATACCTTTATCGACGGCGATATTCCGGGTTTATATCTGGGTAACATTA
CCATGAAAACGGAAGGCACACTCAAGATCGACGCCAAGGGTAATTGGAATTATA
ACGGTGTCGTGCGCGCGTTTAACGATACCTACGACGCTAATCCGTCGACCCACCG
TAGCAAAAGCGCAGAAGATCTGACGACGTTGCTGCGTTTGACGCAAGGTACGCC
GTACGAGATCAGAATCCCGGGTGAGCTGAAGGTGAGCGGTAGCGGCAAGAAGTA
A
25. P835: P628TDRD-P801 FL
Amino acid sequence of P835 (SEQ ID NO: 49):
MSGGDGRGPGNSGLGHNGGQASGNVNGTSGKGGPSSGGGTDPNSGPGWGTTHTPN
GDIHNYNPGEFGNGGSKPGGNGGNSGNHSGSSGGGQSSATAMAFGLPALATPGAEG
LALSVSGDALSAAVADVLAALKGPFKFGLWGIAIYGVLPSEIAKDDPKMMSKIVTSL
PADTVTETPVSSLPLDQATVSVTKRVADIVKDERQHIAVVTGRPMSVPVVDAKPTKR
PGVFSVSIPGLPSLVSVPKGVPAAKAPPKGIIAEKGDSRPAGFTAGGNSREAVIRFPK
ESGQKPVYVSVTDVLTPAQVKQRLEEEKRRQQAWDAAHPEEGLKREYDKAKAELD
AEDKNIATLNSRIASTEKAIPGARAAVQEADKKVKEAEANKDDFVTYNPPHEYGSG
WQDQVRYLDKDIQNQNEKLKAAQTSLNEMNESLSRDKAALSGAMESRKQKEKKA
KDAENKLNEEKSDTMIVVATPTPGFSYASGLTYGGGAFAGAPANGPSEGQIFFQTVL
PAYQSPNLCIGQLAWMTDYINKNGVGNPKTWEVISQNVLIFCSADTALVLNPRIAVY
DGFHKTKWAPAKFNFKTQSQEKFSGNVTTPIAAFGHYLWGEGKPRTVDLSSVGLKI
QANQIDPVMIAVKNNAAGTYQISGNFNRNTFIDGDIPGLYLGNITMKTEGTLKIDAKG
NWNYNGVVRAFNDTYDANPSTHRSKSAEDLTTLLRLTQGTPYEIRIPGELKVSGSGK
K
P835 domain boundaries:
    1-460: P628 TDRD
461-735: P801 full length
Number of amino acids: 735
Molecular weight: 77438
Theoretical pI: 8.68
DNA Sequence of P835: (SEQ ID NO: 50)
ATGAGTGGTGGAGACGGACGAGGTCCGGGTAATTCAGGTCTGGGACACAATGGT
GGTCAGGCCAGTGGGAATGTGAACGGTACATCAGGTAAAGGTGGCCCTTCATCA
GGTGGTGGTACGGATCCAAACAGCGGGCCGGGCTGGGGTACGACGCATACACCT
AACGGGGATATTCATAACTATAACCCGGGGGAGTTTGGTAACGGCGGGAGTAAA
CCCGGTGGTAATGGCGGTAACAGCGGCAATCATAGCGGGAGCTCTGGTGGCGGA
CAGTCTTCGGCCACCGCGATGGCCTTCGGTCTGCCTGCCTTGGCCACTCCGGGGG
CTGAAGGACTGGCTTTATCCGTTTCCGGCGATGCGTTGTCGGCCGCTGTTGCTGA
TGTGCTGGCTGCCCTGAAAGGTCCGTTTAAGTTTGGTCTGTGGGGGATTGCGATT
TACGGAGTGCTGCCTTCTGAGATAGCAAAAGATGATCCGAAAATGATGTCAAAA
ATCGTGACGTCATTGCCGGCAGACACGGTAACGGAGACGCCGGTCAGCTCCCTG
CCGCTGGACCAGGCGACGGTCAGCGTCACTAAACGTGTGGCGGATATTGTGAAG
GACGAGCGGCAGCATATTGCGGTTGTCACCGGCCGGCCAATGAGTGTTCCTGTGG
TGGATGCGAAACCGACAAAACGTCCGGGGGTATTCAGTGTGTCGATTCCGGGTCT
CCCGTCTCTGCAGGTGAGCGTACCTAAAGGTGTTCCGGCCGCGAAAGCCCCGCCA
AAAGGCATTATTGCTGAAAAAGGTGATTCACGTCCGGCTGGTTTTACGGCCGGTG
GTAACTCCCGTGAAGCCGTTATTCGTTTCCCGAAAGAGAGCGGACAGAAACCGG
TCTATGTGTCGGTGACGGATGTTCTTACTCCGGCACAGGTAAAACAGCGTCTGGA
GGAAGAAAAGCGTCGCCAGCAGGCATGGGACGCTGCTCACCCGGAAGAGGGGC
TGAAAAGAGAGTATGATAAAGCGAAAGCTGAGCTGGATGCTGAAGATAAAAAT
ATTGCGACCTTAAACAGTCGCATTGCATCGACAGAGAAGGCGATCCCCGGTGCA
AGGGCTGCTGTTCAGGAAGCCGATAAAAAGGTAAAAGAGGCAGAGGCAAATAA
AGATGATTTTGTGACTTATAACCCGCCTCATGAATATGGCTCCGGGTGGCAGGAT
CAGGTTCGCTATCTTGATAAGGATATTCAGAATCAGAATGAGAAATTAAAAGCT
GCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGC
AGAAAATAAATTAAATGAGGAAAAGTCAGATACAATGATAGTAGTTGCTACTCC
GACCCCGGGTTTTAGCTACGCGAGCGGCTTGACGTACGGCGGTGGTGCGTTTGCG
GGTGCGCCTGCGAACGGTCCGTCGGAAGGCCAAATCTTTTTCCAGACCGTTCTGC
CGGCGTATCAATCCCCGAATCTGTGTATTGGTCAACTGGCTTGGATGACCGATTA
CATTAATAAGAACGGGGTGGGTAACCCAAAAACCTGGGAAGTTATTTCCCAAAA
CGTTCTGATTTTCTGCAGCGCGGATACTGCCCTGGTGCTTAATCCGCGCATCGCG
GTGTACGACGGCTTCCACAAAACTAAATGGGCGCCAGCCAAGTTCAATTTTAAA
ACCCAGAGCCAGGAGAAATTCTCTGGCAACGTGACCACCCCGATTGCTGCCTTCG
GCCATTATCTGTGGGGTGAGGGCAAGCCGCGTACCGTCGACTTGTCTAGCGTTGG
TCTGAAGATCCAGGCAAACCAGATCGACCCGGTTATGATTGCGGTTAAAAACAA
CGCAGCAGGCACCTACCAGATCTCCGGCAACTTCAACCGTAATACCTTTATCGAC
GGCGATATTCCGGGTTTATATCTGGGTAACATTACCATGAAAACGGAAGGCACA
CTCAAGATCGACGCCAAGGGTAATTGGAATTATAACGGTGTCGTGCGCGCGTTTA
ACGATACCTACGACGCTAATCCGTCGACCCACCGTAGCAAAAGCGCAGAAGATC
TGACGACGTTGCTGCGTTTGACGCAAGGTACGCCGTACGAGATCAGAATCCCGG
GTGAGCTGAAGGTGAGCGGTAGCGGCAAGAAGTAA
26. P836 (P628TDRD-4X Rigid linker-P801)
Amino acid sequence of P836 (SEQ ID NO: 51)
MSGGDGRGPGNSGLGHNGGQASGNVNGTSGKGGPSSGGGTDPNSGPGWGTTHTPNGDIHNYNPGEFGNGGSKPGG
NGGNSGNHSGSSGGGQSSATAMAFGLPALATPGAEGLALSVSGDALSAAVADVLAALKGPFKFGLWGIAIYGVLP
SEIAKDDPKMMSKIVTSLPADTVTETPVSSLPLDQATVSVTKRVADIVKDERQHIAVVTGRPMSVPVVDAKPTKR
PGVFSVSIPGLPSLQVSVPKGVPAAKAPPKGIIAEKGDSRPAGFTAGGNSREAVIRFPKESGQKPVYVSVTDVLT
PAQVKQRLEEEKRRQQAWDAAHPEEGLKREYDKAKAELDAEDKNIATLNSRIASTEKAIPGARAAVQEADKKVKE
AEANKDDFVTYNPPHEYGSGWQDQVRYLDKDIQNQNEKLKAAQTSLNEMNESLSRDKAALSGAMESRKQKEKKAK
DAENKLNEEKEAAAKEAAAKEAAAKEAAAKSDTMIVVATPTPGFSYASGLTYGGGAFAGAPANGPSEGQIFFQTV
LPAYQSPNLCIGQLAWMTDYINKNGVGNPKTWEVISQNVLIFCSADTALVLNPRIAVYDGFHKTKWAPAKENFKT
QSQEKFSGNVTTPIAAFGHYLWGEGKPRTVDLSSVGLKIQANQIDPVMIAVKNNAAGTYQISGNFNRNTFIDGDI
PGLYLGNITMKTEGTLKIDAKGNWNYNGVVRAFNDTYDANPSTHRSKSAEDLTTLLRLTQGTPYEIRIPGELKVS
GSGKK
P836 domain boundaries:
    1-460: P628TDRD
461-480: 4X Rigid linker
481-755: P801 full length
Number of amino acids: 755
Molecular weight:79320
Theoretical pI: 8.66
DNA Sequence of P836 (SEQ ID NO: 52)
ATGAGTGGTGGAGACGGACGAGGTCCGGGTAATTCAGGTCTGGGACACAATGGT
GGTCAGGCCAGTGGGAATGTGAACGGTACATCAGGTAAAGGTGGCCCTTCATCA
GGTGGTGGTACGGATCCAAACAGCGGGCCGGGCTGGGGTACGACGCATACACCT
AACGGGGATATTCATAACTATAACCCGGGGGAGTTTGGTAACGGCGGGAGTAAA
CCCGGTGGTAATGGCGGTAACAGCGGCAATCATAGCGGGAGCTCTGGTGGCGGA
CAGTCTTCGGCCACCGCGATGGCCTTCGGTCTGCCTGCCTTGGCCACTCCGGGGG
CTGAAGGACTGGCTTTATCCGTTTCCGGCGATGCGTTGTCGGCCGCTGTTGCTGA
TGTGCTGGCTGCCCTGAAAGGTCCGTTTAAGTTTGGTCTGTGGGGGATTGCGATT
TACGGAGTGCTGCCTTCTGAGATAGCAAAAGATGATCCGAAAATGATGTCAAAA
ATCGTGACGTCATTGCCGGCAGACACGGTAACGGAGACGCCGGTCAGCTCCCTG
CCGCTGGACCAGGCGACGGTCAGCGTCACTAAACGTGTGGCGGATATTGTGAAG
GACGAGCGGCAGCATATTGCGGTTGTCACCGGCCGGCCAATGAGTGTTCCTGTGG
TGGATGCGAAACCGACAAAACGTCCGGGGGTATTCAGTGTGTCGATTCCGGGTCT
CCCGTCTCTGCAGGTGAGCGTACCTAAAGGTGTTCCGGCCGCGAAAGCCCCGCCA
AAAGGCATTATTGCTGAAAAAGGTGATTCACGTCCGGCTGGTTTTACGGCCGGTG
GTAACTCCCGTGAAGCCGTTATTCGTTTCCCGAAAGAGAGCGGACAGAAACCGG
TCTATGTGTCGGTGACGGATGTTCTTACTCCGGCACAGGTAAAACAGCGTCTGGA
GGAAGAAAAGCGTCGCCAGCAGGCATGGGACGCTGCTCACCCGGAAGAGGGGC
TGAAAAGAGAGTATGATAAAGCGAAAGCTGAGCTGGATGCTGAAGATAAAAAT
ATTGCGACCTTAAACAGTCGCATTGCATCGACAGAGAAGGCGATCCCCGGTGCA
AGGGCTGCTGTTCAGGAAGCCGATAAAAAGGTAAAAGAGGCAGAGGCAAATAA
AGATGATTTTGTGACTTATAACCCGCCTCATGAATATGGCTCCGGGTGGCAGGAT
CAGGTTCGCTATCTTGATAAGGATATTCAGAATCAGAATGAGAAATTAAAAGCT
GCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGC
AGAAAATAAATTAAATGAGGAAAAGGAAGCCGCTGCCAAGGAGGCGGCCGCTA
AAGAAGCCGCTGCCAAGGAGGCGGCCGCTAAATCAGATACAATGATAGTAGTTG
CTACTCCGACCCCGGGTTTTAGCTACGCGAGCGGCTTGACGTACGGCGGTGGTGC
GTTTGCGGGTGCGCCTGCGAACGGTCCGTCGGAAGGCCAAATCTTTTTCCAGACC
GTTCTGCCGGCGTATCAATCCCCGAATCTGTGTATTGGTCAACTGGCTTGGATGA
CCGATTACATTAATAAGAACGGGGTGGGTAACCCAAAAACCTGGGAAGTTATTT
CCCAAAACGTTCTGATTTTCTGCAGCGCGGATACTGCCCTGGTGCTTAATCCGCG
CATCGCGGTGTACGACGGCTTCCACAAAACTAAATGGGCGCCAGCCAAGTTCAA
TTTTAAAACCCAGAGCCAGGAGAAATTCTCTGGCAACGTGACCACCCCGATTGCT
GCCTTCGGCCATTATCTGTGGGGTGAGGGCAAGCCGCGTACCGTCGACTTGTCTA
GCGTTGGTCTGAAGATCCAGGCAAACCAGATCGACCCGGTTATGATTGCGGTTAA
AAACAACGCAGCAGGCACCTACCAGATCTCCGGCAACTTCAACCGTAATACCTTT
ATCGACGGCGATATTCCGGGTTTATATCTGGGTAACATTACCATGAAAACGGAAG
GCACACTCAAGATCGACGCCAAGGGTAATTGGAATTATAACGGTGTCGTGCGCG
CGTTTAACGATACCTACGACGCTAATCCGTCGACCCACCGTAGCAAAAGCGCAG
AAGATCTGACGACGTTGCTGCGTTTGACGCAAGGTACGCCGTACGAGATCAGAA
TCCCGGGTGAGCTGAAGGTGAGCGGTAGCGGCAAGAAGTAA
27. P837: P628 TDRD-FL-P801FL
Amino acid sequence of P837 (SEQ ID NO: 53)
MSGGDGRGPGNSGLGHNGGQASGNVNGTSGKGGPSSGGGTDPNSGPGWGTTHTPN
GDIHNYNPGEFGNGGSKPGGNGGNSGNHSGSSGGGQSSATAMAFGLPALATPGAEG
LALSVSGDALSAAVADVLAALKGPFKFGLWGIAIYGVLPSEIAKDDPKMMSKIVTSL
PADTVTETPVSSLPLDQATVSVTKRVADIVKDERQHIAVVTGRPMSVPVVDAKPTKR
PGVFSVSIPGLPSLQVSVPKGVPAAKAPPKGIIAEKGDSRPAGFTAGGNSREAVIRFPK
ESGQKPVYVSVTDVLTPAQVKQRLEEEKRRQQAWDAAHPEEGLKREYDKAKAELD
AEDKNIATLNSRIASTEKAIPGARAAVQEADKKVKEAEANKDDFVTYNPPHEYGSG
WQDQVRYLDKDIQNQNEKLKAAQTSLNEMNESLSRDKAALSGAMESRKQKEKKA
KDAENKLNEEKGSGSASGSDTMIVVATPTPGFSYASGLTYGGGAFAGAPANGPSEG
QIFFQTVLPAYQSPNLCIGQLAWMTDYINKNGVGNPKTWEVISQNVLIFCSADTALV
LNPRIAVYDGFHKTKWAPAKFNFKTQSQEKFSGNVTTPIAAFGHYLWGEGKPRTVD
LSSVGLKIQANQIDPVMIAVKNNAAGTYQISGNFNRNTFIDGDIPGLYLGNITMKTEG
TLKIDAKGNWNYNGVVRAFNDTYDANPSTHRSKSAEDLTTLLRLTQGTPYEIRIPGE
LKVSGSGKK
P837 domain boundaries:
    1-460: P628 TDRD
461-467: Flexible linker
468-742: P801 full length
Number of amino acids: 742
Molecular weight: 77942
Theoretical pI: 8.68
DNA Sequence of P837 (SEQ ID NO: 54)
ATGAGTGGTGGAGACGGACGAGGTCCGGGTAATTCAGGTCTGGGACACAATGGT
GGTCAGGCCAGTGGGAATGTGAACGGTACATCAGGTAAAGGTGGCCCTTCATCA
GGTGGTGGTACGGATCCAAACAGCGGGCCGGGCTGGGGTACGACGCATACACCT
AACGGGGATATTCATAACTATAACCCGGGGGAGTTTGGTAACGGCGGGAGTAAA
CCCGGTGGTAATGGCGGTAACAGCGGCAATCATAGCGGGAGCTCTGGTGGCGGA
CAGTCTTCGGCCACCGCGATGGCCTTCGGTCTGCCTGCCTTGGCCACTCCGGGGG
CTGAAGGACTGGCTTTATCCGTTTCCGGCGATGCGTTGTCGGCCGCTGTTGCTGA
TGTGCTGGCTGCCCTGAAAGGTCCGTTTAAGTTTGGTCTGTGGGGGATTGCGATT
TACGGAGTGCTGCCTTCTGAGATAGCAAAAGATGATCCGAAAATGATGTCAAAA
ATCGTGACGTCATTGCCGGCAGACACGGTAACGGAGACGCCGGTCAGCTCCCTG
CCGCTGGACCAGGCGACGGTCAGCGTCACTAAACGTGTGGCGGATATTGTGAAG
GACGAGCGGCAGCATATTGCGGTTGTCACCGGCCGGCCAATGAGTGTTCCTGTGG
TGGATGCGAAACCGACAAAACGTCCGGGGGTATTCAGTGTGTCGATTCCGGGTCT
CCCGTCTCTGCAGGTGAGCGTACCTAAAGGTGTTCCGGCCGCGAAAGCCCCGCCA
AAAGGCATTATTGCTGAAAAAGGTGATTCACGTCCGGCTGGTTTTACGGCCGGTG
GTAACTCCCGTGAAGCCGTTATTCGTTTCCCGAAAGAGAGCGGACAGAAACCGG
TCTATGTGTCGGTGACGGATGTTCTTACTCCGGCACAGGTAAAACAGCGTCTGGA
GGAAGAAAAGCGTCGCCAGCAGGCATGGGACGCTGCTCACCCGGAAGAGGGGC
TGAAAAGAGAGTATGATAAAGCGAAAGCTGAGCTGGATGCTGAAGATAAAAAT
ATTGCGACCTTAAACAGTCGCATTGCATCGACAGAGAAGGCGATCCCCGGTGCA
AGGGCTGCTGTTCAGGAAGCCGATAAAAAGGTAAAAGAGGCAGAGGCAAATAA
AGATGATTTTGTGACTTATAACCCGCCTCATGAATATGGCTCCGGGTGGCAGGAT
CAGGTTCGCTATCTTGATAAGGATATTCAGAATCAGAATGAGAAATTAAAAGCT
GCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGC
AGAAAATAAATTAAATGAGGAAAAGGGCTCCGGATCGGCTTCTGGGTCAGATAC
AATGATAGTAGTTGCTACTCCGACCCCGGGTTTTAGCTACGCGAGCGGCTTGACG
TACGGCGGTGGTGCGTTTGCGGGTGCGCCTGCGAACGGTCCGTCGGAAGGCCAA
ATCTTTTTCCAGACCGTTCTGCCGGCGTATCAATCCCCGAATCTGTGTATTGGTCA
ACTGGCTTGGATGACCGATTACATTAATAAGAACGGGGTGGGTAACCCAAAAAC
CTGGGAAGTTATTTCCCAAAACGTTCTGATTTTCTGCAGCGCGGATACTGCCCTG
GTGCTTAATCCGCGCATCGCGGTGTACGACGGCTTCCACAAAACTAAATGGGCGC
CAGCCAAGTTCAATTTTAAAACCCAGAGCCAGGAGAAATTCTCTGGCAACGTGA
CCACCCCGATTGCTGCCTTCGGCCATTATCTGTGGGGTGAGGGCAAGCCGCGTAC
CGTCGACTTGTCTAGCGTTGGTCTGAAGATCCAGGCAAACCAGATCGACCCGGTT
ATGATTGCGGTTAAAAACAACGCAGCAGGCACCTACCAGATCTCCGGCAACTTC
AACCGTAATACCTTTATCGACGGCGATATTCCGGGTTTATATCTGGGTAACATTA
CCATGAAAACGGAAGGCACACTCAAGATCGACGCCAAGGGTAATTGGAATTATA
ACGGTGTCGTGCGCGCGTTTAACGATACCTACGACGCTAATCCGTCGACCCACCG
TAGCAAAAGCGCAGAAGATCTGACGACGTTGCTGCGTTTGACGCAAGGTACGCC
GTACGAGATCAGAATCCCGGGTGAGCTGAAGGTGAGCGGTAGCGGCAAGAAGTA
A
28. P842: P628 FL-P801FL
Amino acid sequence of P842 (SEQ ID NO: 55)
MSGGDGRGPGNSGLGHNGGQASGNVNGTSGKGGPSSGGGTDPNSGPGWGTTHTPN
GDIHNYNPGEFGNGGSKPGGNGGNSGNHSGSSGGGQSSATAMAFGLPALATPGAEG
LALSVSGDALSAAVADVLAALKGPFKFGLWGIAIYGVLPSEIAKDDPKMMSKIVTSL
PADTVTETPVSSLPLDQATVSVTKRVADIVKDERQHIAVVTGRPMSVPVVDAKPTKR
PGVFSVSIPGLPSLQVSVPKGVPAAKAPPKGIIAEKGDSRPAGFTAGGNSREAVIRFPK
ESGQKPVYVSVTDVLTPAQVKQRLEEEKRRQQAWDAAHPEEGLKREYDKAKAELD
AEDKNIATLNSRIASTEKAIPGARAAVQEADKKVKEAEANKDDFVTYNPPHEYGSG
WQDQVRYLDKDIQNQNEKLKAAQTSLNEMNESLSRDKAALSGAMESRKQKEKKA
KDAENKLNEEKKKPRKGTKDYGHDYFPDPKTEDIKGLGELKEGKPKTPKQGGGGKR
ARWYGDKKRKIYEWDSQHGELEGYRASDGEHLGAFDPKTGKQVKGPDPKRNIKKY
LSDTMIVVATPTPGFSYASGLTYGGGAFAGAPANGPSEGQIFFQTVLPAYQSPNLCIG
QLAWMTDYINKNGVGNPKTWEVISQNVLIFCSADTALVLNPRIAVYDGFHKTKWAP
AKFNFKTQSQEKFSGNVTTPIAAFGHYLWGEGKPRTVDLSSVGLKIQANQIDPVMIA
VKNNAAGTYQISGNFNRNTFIDGDIPGLYLGNITMKTEGTLKIDAKGNWNYNGVVR
AFNDTYDANPSTHRSKSAEDLTTLLRLTQGTPYEIRIPGELKVSGSGKK
P842 domain boundaries:
    1-561: P628 full length
562-836: P801 full length
Number of amino acids: 836
Molecular weight: 88920
Theoretical pI: 9.17
DNA Sequence of P842: (SEQ ID NO: 56)
ATGAGTGGTGGAGACGGACGAGGTCCGGGTAATTCAGGTCTGGGACACAATGGT
GGTCAGGCCAGTGGGAATGTGAACGGTACATCAGGTAAAGGTGGCCCTTCATCA
GGTGGTGGTACGGATCCAAACAGCGGGCCGGGCTGGGGTACGACGCATACACCT
AACGGGGATATTCATAACTATAACCCGGGGGAGTTTGGTAACGGCGGGAGTAAA
CCCGGTGGTAATGGCGGTAACAGCGGCAATCATAGCGGGAGCTCTGGTGGCGGA
CAGTCTTCGGCCACCGCGATGGCCTTCGGTCTGCCTGCCTTGGCCACTCCGGGGG
CTGAAGGACTGGCTTTATCCGTTTCCGGCGATGCGTTGTCGGCCGCTGTTGCTGA
TGTGCTGGCTGCCCTGAAAGGTCCGTTTAAGTTTGGTCTGTGGGGGATTGCGATT
TACGGAGTGCTGCCTTCTGAGATAGCAAAAGATGATCCGAAAATGATGTCAAAA
ATCGTGACGTCATTGCCGGCAGACACGGTAACGGAGACGCCGGTCAGCTCCCTG
CCGCTGGACCAGGCGACGGTCAGCGTCACTAAACGTGTGGCGGATATTGTGAAG
GACGAGCGGCAGCATATTGCGGTTGTCACCGGCCGGCCAATGAGTGTTCCTGTGG
TGGATGCGAAACCGACAAAACGTCCGGGGGTATTCAGTGTGTCGATTCCGGGTCT
CCCGTCTCTGCAGGTGAGCGTACCTAAAGGTGTTCCGGCCGCGAAAGCCCCGCCA
AAAGGCATTATTGCTGAAAAAGGTGATTCACGTCCGGCTGGTTTTACGGCCGGTG
GTAACTCCCGTGAAGCCGTTATTCGTTTCCCGAAAGAGAGCGGACAGAAACCGG
TCTATGTGTCGGTGACGGATGTTCTTACTCCGGCACAGGTAAAACAGCGTCTGGA
GGAAGAAAAGCGTCGCCAGCAGGCATGGGACGCTGCTCACCCGGAAGAGGGGC
TGAAAAGAGAGTATGATAAAGCGAAAGCTGAGCTGGATGCTGAAGATAAAAAT
ATTGCGACCTTAAACAGTCGCATTGCATCGACAGAGAAGGCGATCCCCGGTGCA
AGGGCTGCTGTTCAGGAAGCCGATAAAAAGGTAAAAGAGGCAGAGGCAAATAA
AGATGATTTTGTGACTTATAACCCGCCTCATGAATATGGCTCCGGGTGGCAGGAT
CAGGTTCGCTATCTTGATAAGGATATTCAGAATCAGAATGAGAAATTAAAAGCT
GCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGC
AGAAAATAAATTAAATGAGGAAAAGAAAAAACCTCGTAAGGGAACTAAAGATT
ACGGCCATGATTATTTTCCTGATCCCAAGACTGAAGATATTAAGGGGTTGGGAGA
GTTGAAAGAGGGTAAACCTAAAACCCCTAAACAAGGTGGTGGTGGTAAGCGGGC
CCGATGGTATGGTGATAAAAAGCGTAAAATTTATGAATGGGATTCCCAGCACGG
TGAGCTTGAAGGGTACCGCGCCAGTGATGGCGAACACCTCGGGGCATTCGATCC
AAAAACGGGTAAGCAGGTTAAAGGGCCGGATCCAAAACGAAACATTAAAAAAT
ATCTTGGCTCCGGATCGGCTTCTGGGTCAGATACAATGATAGTAGTTGCTACTCC
GACCCCGGGTTTTAGCTACGCGAGCGGCTTGACGTACGGCGGTGGTGCGTTTGCG
GGTGCGCCTGCGAACGGTCCGTCGGAAGGCCAAATCTTTTTCCAGACCGTTCTGC
CGGCGTATCAATCCCCGAATCTGTGTATTGGTCAACTGGCTTGGATGACCGATTA
CATTAATAAGAACGGGGTGGGTAACCCAAAAACCTGGGAAGTTATTTCCCAAAA
CGTTCTGATTTTCTGCAGCGCGGATACTGCCCTGGTGCTTAATCCGCGCATCGCG
GTGTACGACGGCTTCCACAAAACTAAATGGGCGCCAGCCAAGTTCAATTTTAAA
ACCCAGAGCCAGGAGAAATTCTCTGGCAACGTGACCACCCCGATTGCTGCCTTCG
GCCATTATCTGTGGGGTGAGGGCAAGCCGCGTACCGTCGACTTGTCTAGCGTTGG
TCTGAAGATCCAGGCAAACCAGATCGACCCGGTTATGATTGCGGTTAAAAACAA
CGCAGCAGGCACCTACCAGATCTCCGGCAACTTCAACCGTAATACCTTTATCGAC
GGCGATATTCCGGGTTTATATCTGGGTAACATTACCATGAAAACGGAAGGCACA
CTCAAGATCGACGCCAAGGGTAATTGGAATTATAACGGTGTCGTGCGCGCGTTTA
ACGATACCTACGACGCTAATCCGTCGACCCACCGTAGCAAAAGCGCAGAAGATC
TGACGACGTTGCTGCGTTTGACGCAAGGTACGCCGTACGAGATCAGAATCCCGG
GTGAGCTGAAGGTGAGCGGTAGCGGCAAGAAGTAA
29. P845: P628FL-FL-P801FL
Amino acid sequence of P845 (SEQ ID NO: 57)
MSGGDGRGPGNSGLGHNGGQASGNVNGTSGKGGPSSGGGTDPNSGPGWGTTHTPN
GDIHNYNPGEFGNGGSKPGGNGGNSGNHSGSSGGGQSSATAMAFGLPALATPGAEG
LALSVSGDALSAAVADVLAALKGPFKFGLWGIAIYGVLPSEIAKDDPKMMSKIVTSL
PADTVTETPVSSLPLDQATVSVTKRVADIVKDERQHIAVVTGRPMSVPVVDAKPTKR
PGVFSVSIPGLPSLQVSVPKGVPAAKAPPKGIIAEKGDSRPAGFTAGGNSREAVIRFPK
ESGQKPVYVSVTDVLTPAQVKQRLEEEKRRQQAWDAAHPEEGLKREYDKAKAELD
AEDKNIATLNSRIASTEKAIPGARAAVQEADKKVKEAEANKDDFVTYNPPHEYGSG
WQDQVRYLDKDIQNQNEKLKAAQTSLNEMNESLSRDKAALSGAMESRKQKEKKA
KDAENKLNEEKKKPRKGTKDYGHDYFPDPKTEDIKGLGELKEGKPKTPKQGGGGKR
ARWYGDKKRKIYEWDSQHGELEGYRASDGEHLGAFDPKTGKQVKGPDPKRNIKKY
LGSGSASGSDTMIVVATPTPGFSYASGLTYGGGAFAGAPANGPSEGQIFFQTVLPAYQ
SPNLCIGQLAWMTDYINKNGVGNPKTWEVISQNVLIFCSADTALVLNPRIAVYDGFH
KTKWAPAKFNFKTQSQEKFSGNVTTPIAAFGHYLWGEGKPRTVDLSSVGLKIQANQI
DPVMIAVKNNAAGTYQISGNFNRNTFIDGDIPGLYLGNITMKTEGTLKIDAKGNWNY
NGVVRAFNDTYDANPSTHRSKSAEDLTTLLRLTQGTPYEIRIPGELKVSGSGKK
P845 domain boundaries:
    1-561: P628 Full length
562-568: Flexible linker
569-843: P801 full length
Number of amino acids: 843
Molecular weight: 89424
Theoretical pI: 9.17
DNA Sequence of P845 (SEQ ID NO: 58)
ATGAGTGGTGGAGACGGACGAGGTCCGGGTAATTCAGGTCTGGGACACAATGGT
GGTCAGGCCAGTGGGAATGTGAACGGTACATCAGGTAAAGGTGGCCCTTCATCA
GGTGGTGGTACGGATCCAAACAGCGGGCCGGGCTGGGGTACGACGCATACACCT
AACGGGGATATTCATAACTATAACCCGGGGGAGTTTGGTAACGGCGGGAGTAAA
CCCGGTGGTAATGGCGGTAACAGCGGCAATCATAGCGGGAGCTCTGGTGGCGGA
CAGTCTTCGGCCACCGCGATGGCCTTCGGTCTGCCTGCCTTGGCCACTCCGGGGG
CTGAAGGACTGGCTTTATCCGTTTCCGGCGATGCGTTGTCGGCCGCTGTTGCTGA
TGTGCTGGCTGCCCTGAAAGGTCCGTTTAAGTTTGGTCTGTGGGGGATTGCGATT
TACGGAGTGCTGCCTTCTGAGATAGCAAAAGATGATCCGAAAATGATGTCAAAA
ATCGTGACGTCATTGCCGGCAGACACGGTAACGGAGACGCCGGTCAGCTCCCTG
CCGCTGGACCAGGCGACGGTCAGCGTCACTAAACGTGTGGCGGATATTGTGAAG
GACGAGCGGCAGCATATTGCGGTTGTCACCGGCCGGCCAATGAGTGTTCCTGTGG
TGGATGCGAAACCGACAAAACGTCCGGGGGTATTCAGTGTGTCGATTCCGGGTCT
CCCGTCTCTGCAGGTGAGCGTACCTAAAGGTGTTCCGGCCGCGAAAGCCCCGCCA
AAAGGCATTATTGCTGAAAAAGGTGATTCACGTCCGGCTGGTTTTACGGCCGGTG
GTAACTCCCGTGAAGCCGTTATTCGTTTCCCGAAAGAGAGCGGACAGAAACCGG
TCTATGTGTCGGTGACGGATGTTCTTACTCCGGCACAGGTAAAACAGCGTCTGGA
GGAAGAAAAGCGTCGCCAGCAGGCATGGGACGCTGCTCACCCGGAAGAGGGGC
TGAAAAGAGAGTATGATAAAGCGAAAGCTGAGCTGGATGCTGAAGATAAAAAT
ATTGCGACCTTAAACAGTCGCATTGCATCGACAGAGAAGGCGATCCCCGGTGCA
AGGGCTGCTGTTCAGGAAGCCGATAAAAAGGTAAAAGAGGCAGAGGCAAATAA
AGATGATTTTGTGACTTATAACCCGCCTCATGAATATGGCTCCGGGTGGCAGGAT
CAGGTTCGCTATCTTGATAAGGATATTCAGAATCAGAATGAGAAATTAAAAGCT
GCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGC
AGAAAATAAATTAAATGAGGAAAAGAAAAAACCTCGTAAGGGAACTAAAGATT
ACGGCCATGATTATTTTCCTGATCCCAAGACTGAAGATATTAAGGGGTTGGGAGA
GTTGAAAGAGGGTAAACCTAAAACCCCTAAACAAGGTGGTGGTGGTAAGCGGGC
CCGATGGTATGGTGATAAAAAGCGTAAAATTTATGAATGGGATTCCCAGCACGG
TGAGCTTGAAGGGTACCGCGCCAGTGATGGCGAACACCTCGGGGCATTCGATCC
AAAAACGGGTAAGCAGGTTAAAGGGCCGGATCCAAAACGAAACATTAAAAAAT
ATCTTGGCTCCGGATCGGCTTCTGGGTCAGATACAATGATAGTAGTTGCTACTCC
GACCCCGGGTTTTAGCTACGCGAGCGGCTTGACGTACGGCGGTGGTGCGTTTGCG
GGTGCGCCTGCGAACGGTCCGTCGGAAGGCCAAATCTTTTTCCAGACCGTTCTGC
CGGCGTATCAATCCCCGAATCTGTGTATTGGTCAACTGGCTTGGATGACCGATTA
CATTAATAAGAACGGGGTGGGTAACCCAAAAACCTGGGAAGTTATTTCCCAAAA
CGTTCTGATTTTCTGCAGCGCGGATACTGCCCTGGTGCTTAATCCGCGCATCGCG
GTGTACGACGGCTTCCACAAAACTAAATGGGCGCCAGCCAAGTTCAATTTTAAA
ACCCAGAGCCAGGAGAAATTCTCTGGCAACGTGACCACCCCGATTGCTGCCTTCG
GCCATTATCTGTGGGGTGAGGGCAAGCCGCGTACCGTCGACTTGTCTAGCGTTGG
TCTGAAGATCCAGGCAAACCAGATCGACCCGGTTATGATTGCGGTTAAAAACAA
CGCAGCAGGCACCTACCAGATCTCCGGCAACTTCAACCGTAATACCTTTATCGAC
GGCGATATTCCGGGTTTATATCTGGGTAACATTACCATGAAAACGGAAGGCACA
CTCAAGATCGACGCCAAGGGTAATTGGAATTATAACGGTGTCGTGCGCGCGTTTA
ACGATACCTACGACGCTAATCCGTCGACCCACCGTAGCAAAAGCGCAGAAGATC
TGACGACGTTGCTGCGTTTGACGCAAGGTACGCCGTACGAGATCAGAATCCCGG
GTGAGCTGAAGGTGAGCGGTAGCGGCAAGAAGTAA
30. P862 (P628FL-Rigid Linker-P801FL)
Amino acid sequence of P862 (SEQ ID NO: 59)
MSGGDGRGPGNSGLGHNGGQASGNVNGTSGKGGPSSGGGTDPNSGPGWGTTHTPN
GDIHNYNPGEFGNGGSKPGGNGGNSGNHSGSSGGGQSSATAMAFGLPALATPGAEG
LALSVSGDALSAAVADVLAALKGPFKFGLWGIAIYGVLPSEIAKDDPKMMSKIVTSL
PADTVTETPVSSLPLDQATVSVTKRVADIVKDERQHIAVVTGRPMSVPVVDAKPTKR
PGVFSVSIPGLPSLQVSVPKGVPAAKAPPKGIIAEKGDSRPAGFTAGGNSREAVIRFPK
ESGQKPVYVSVTDVLTPAQVKQRLEEEKRRQQAWDAAHPEEGLKREYDKAKAELD
AEDKNIATLNSRIASTEKAIPGARAAVQEADKKVKEAEANKDDFVTYNPPHEYGSG
WQDQVRYLDKDIQNQNEKLKAAQTSLNEMNESLSRDKAALSGAMESRKQKEKKA
KDAENKLNEEKKKPRKGTKDYGHDYFPDPKTEDIKGLGELKEGKPKTPKQGGGGKR
ARWYGDKKRKIYEWDSQHGELEGYRASDGEHLGAFDPKTGKQVKGPDPKRNIKKY
LEAAAKEAAAKSDTMIVVATPTPGFSYASGLTYGGGAFAGAPANGPSEGQIFFQTVL
PAYQSPNLCIGQLAWMTDYINKNGVGNPKTWEVISQNVLIFCSADTALVLNPRIAVY
DGFHKTKWAPAKFNFKTQSQEKFSGNVTTPIAAFGHYLWGEGKPRTVDLSSVGLKI
QANQIDPVMIAVKNNAAGTYQISGNFNRNTFIDGDIPGLYLGNITMKTEGTLKIDAKG
NWNYNGVVRAFNDTYDANPSTHRSKSAEDLTTLLRLTQGTPYEIRIPGELKVSGSGK
K
P862 domain boundaries:
    1-561: P628 Full length
562-571: Rigid linker
572-846: P801 full length
Number of amino acids: 846
Molecular weight: 89861
Theoretical pI: 9.16
DNA Sequence of P862 (SEQ ID NO: 60)
ATGAGTGGTGGAGACGGACGAGGTCCGGGTAATTCAGGTCTGGGACACAATGGT
GGTCAGGCCAGTGGGAATGTGAACGGTACATCAGGTAAAGGTGGCCCTTCATCA
GGTGGTGGTACGGATCCAAACAGCGGGCCGGGCTGGGGTACGACGCATACACCT
AACGGGGATATTCATAACTATAACCCGGGGGAGTTTGGTAACGGCGGGAGTAAA
CCCGGTGGTAATGGCGGTAACAGCGGCAATCATAGCGGGAGCTCTGGTGGCGGA
CAGTCTTCGGCCACCGCGATGGCCTTCGGTCTGCCTGCCTTGGCCACTCCGGGGG
CTGAAGGACTGGCTTTATCCGTTTCCGGCGATGCGTTGTCGGCCGCTGTTGCTGA
TGTGCTGGCTGCCCTGAAAGGTCCGTTTAAGTTTGGTCTGTGGGGGATTGCGATT
TACGGAGTGCTGCCTTCTGAGATAGCAAAAGATGATCCGAAAATGATGTCAAAA
ATCGTGACGTCATTGCCGGCAGACACGGTAACGGAGACGCCGGTCAGCTCCCTG
CCGCTGGACCAGGCGACGGTCAGCGTCACTAAACGTGTGGCGGATATTGTGAAG
GACGAGCGGCAGCATATTGCGGTTGTCACCGGCCGGCCAATGAGTGTTCCTGTGG
TGGATGCGAAACCGACAAAACGTCCGGGGGTATTCAGTGTGTCGATTCCGGGTCT
CCCGTCTCTGCAGGTGAGCGTACCTAAAGGTGTTCCGGCCGCGAAAGCCCCGCCA
AAAGGCATTATTGCTGAAAAAGGTGATTCACGTCCGGCTGGTTTTACGGCCGGTG
GTAACTCCCGTGAAGCCGTTATTCGTTTCCCGAAAGAGAGCGGACAGAAACCGG
TCTATGTGTCGGTGACGGATGTTCTTACTCCGGCACAGGTAAAACAGCGTCTGGA
GGAAGAAAAGCGTCGCCAGCAGGCATGGGACGCTGCTCACCCGGAAGAGGGGC
TGAAAAGAGAGTATGATAAAGCGAAAGCTGAGCTGGATGCTGAAGATAAAAAT
ATTGCGACCTTAAACAGTCGCATTGCATCGACAGAGAAGGCGATCCCCGGTGCA
AGGGCTGCTGTTCAGGAAGCCGATAAAAAGGTAAAAGAGGCAGAGGCAAATAA
AGATGATTTTGTGACTTATAACCCGCCTCATGAATATGGCTCCGGGTGGCAGGAT
CAGGTTCGCTATCTTGATAAGGATATTCAGAATCAGAATGAGAAATTAAAAGCT
GCCCAGACATCTTTAAACGAAATGAATGAATCCTTATCCAGGGATAAGGCTGCG
CTTTCCGGGGCGATGGAGAGCCGGAAACAAAAGGAGAAAAAAGCGAAGGATGC
AGAAAATAAATTAAATGAGGAAAAGAAAAAACCTCGTAAGGGAACTAAAGATT
ACGGCCATGATTATTTTCCTGATCCCAAGACTGAAGATATTAAGGGGTTGGGAGA
GTTGAAAGAGGGTAAACCTAAAACCCCTAAACAAGGTGGTGGTGGTAAGCGGGC
CCGATGGTATGGTGATAAAAAGCGTAAAATTTATGAATGGGATTCCCAGCACGG
TGAGCTTGAAGGGTACCGCGCCAGTGATGGCGAACACCTCGGGGCATTCGATCC
AAAAACGGGTAAGCAGGTTAAAGGGCCGGATCCAAAACGAAACATTAAAAAAT
ATCTTGAAGCCGCTGCCAAGGAGGCGGCCGCTAAATCAGATACAATGATAGTAG
TTGCTACTCCGACCCCGGGTTTTAGCTACGCGAGCGGCTTGACGTACGGCGGTGG
TGCGTTTGCGGGTGCGCCTGCGAACGGTCCGTCGGAAGGCCAAATCTTTTTCCAG
ACCGTTCTGCCGGCGTATCAATCCCCGAATCTGTGTATTGGTCAACTGGCTTGGA
TGACCGATTACATTAATAAGAACGGGGTGGGTAACCCAAAAACCTGGGAAGTTA
TTTCCCAAAACGTTCTGATTTTCTGCAGCGCGGATACTGCCCTGGTGCTTAATCCG
CGCATCGCGGTGTACGACGGCTTCCACAAAACTAAATGGGCGCCAGCCAAGTTC
AATTTTAAAACCCAGAGCCAGGAGAAATTCTCTGGCAACGTGACCACCCCGATT
GCTGCCTTCGGCCATTATCTGTGGGGTGAGGGCAAGCCGCGTACCGTCGACTTGT
CTAGCGTTGGTCTGAAGATCCAGGCAAACCAGATCGACCCGGTTATGATTGCGGT
TAAAAACAACGCAGCAGGCACCTACCAGATCTCCGGCAACTTCAACCGTAATAC
CTTTATCGACGGCGATATTCCGGGTTTATATCTGGGTAACATTACCATGAAAACG
GAAGGCACACTCAAGATCGACGCCAAGGGTAATTGGAATTATAACGGTGTCGTG
CGCGCGTTTAACGATACCTACGACGCTAATCCGTCGACCCACCGTAGCAAAAGC
GCAGAAGATCTGACGACGTTGCTGCGTTTGACGCAAGGTACGCCGTACGAGATC
AGAATCCCGGGTGAGCTGAAGGTGAGCGGTAGCGGCAAGAAGTAA
31. P863 (P764FL-Rigid Linker-P801FL)
Amino acid sequence of P862 (SEQ ID NO: 61)
MPEETLTVVGGGNNSCNVSWGGGNGNNGGAGYSGKYGGTSYEGATSMLKLNDRV
LIQLYLCNPLNPDYIGAPWGSDKDAESIIRANRDKAGKFKANIQNWKTSGTGSLGSP
VVGKSYSSGDVDTYSVSFGKEKYNVLYNRKKDSFTTAYVDGGANKPEHSMKDQAI
AVVKLYLLNESQASVIDTTSGIITDSGKTLSGKLGDKYNTLAKEAADNIKNFQGKKL
RSFNDAMASINELANNPKMKLSQADKTVVSNALKQMDLSALADRFKGLEKAFTWG
DRLLKAEKIREGVVTGITTGDWQKLAFEVEAMYLSGVAGAVALGITTAMISTVAVA
LSLPAVAVSALTVVSVIGISILTSYIDADKAKALNNAVLGLFKEAAAKEAAAKSDTMI
VVATPTPGFSYASGLTYGGGAFAGAPANGPSEGQIFFQTVLPAYQSPNLCIGQLAWM
TDYINKNGVGNPKTWEVISQNVLIFCSADTALVLNPRIAVYDGFHKTKWAPAKFNFK
TQSQEKFSGNVTTPIAAFGHYLWGEGKPRTVDLSSVGLKIQANQIDPVMIAVKNNAA
GTYQISGNFNRNTFIDGDIPGLYLGNITMKTEGTLKIDAKGNWNYNGVVRAFNDTYD
ANPSTHRSKSAEDLTTLLRLTQGTPYEIRIPGELKVSGSGKK
P863 domain boundaries:
    1-377: P764 Full length
378-387: Rigid linker
388-662: P801 full length
Number of amino acids: 662
Molecular weight: 70505
Theoretical pI: 8.99
DNA Sequence of P863 (SEQ ID NO: 62)
ATGCCTGAAGAAACATTGACTGTCGTAGGTGGTGGTAATAACAGCTGTAATGTTA
GCTGGGGTGGTGGCAATGGTAACAACGGTGGTGCTGGCTATTCTGGTAAATACG
GTGGTACTAGCTATGAAGGTGCAACTAGTATGTTGAAGTTGAATGACCGTGTTTT
AATTCAGCTTTATCTTTGCAATCCGCTTAACCCAGATTATATTGGAGCACCTTGGG
GCAGTGATAAAGATGCAGAATCAATTATCAGAGCTAACAGAGATAAAGCAGGAA
AGTTCAAGGCTAATATTCAGAACTGGAAAACAAGCGGTACAGGTTCTTTAGGTA
GCCCTGTAGTTGGGAAATCTTATAGCTCAGGTGATGTAGATACTTATTCGGTTAG
CTTCGGAAAAGAAAAATACAATGTCCTGTATAACCGTAAAAAAGACTCTTTTACT
ACAGCTTATGTCGATGGCGGCGCAAATAAACCTGAGCATAGCATGAAGGATCAG
GCTATTGCCGTTGTTAAACTTTACCTTCTCAACGAAAGTCAGGCATCGGTAATCG
ATACTACATCGGGAATTATTACTGACTCTGGTAAAACTCTTAGCGGGAAATTAGG
TGATAAATACAACACTCTGGCGAAAGAAGCTGCTGACAATATAAAAAACTTCCA
GGGTAAGAAACTCCGCAGTTTTAATGATGCTATGGCATCTATTAATGAACTAGCT
AACAATCCAAAGATGAAGTTAAGTCAGGCGGATAAAACAGTCGTTTCTAATGCC
CTCAAACAAATGGATTTGTCAGCACTAGCTGACCGATTCAAAGGGTTAGAGAAA
GCCTTTACTTGGGGTGATCGACTTCTTAAAGCCGAGAAAATCAGAGAAGGTGTTG
TTACTGGTATTACCACAGGGGACTGGCAAAAGCTGGCGTTTGAGGTTGAAGCTAT
GTACCTCAGTGGTGTTGCTGGCGCCGTAGCGTTAGGGATTACTACTGCCATGATT
AGCACAGTCGCAGTCGCTTTGTCACTTCCAGCTGTAGCTGTCTCTGCGCTTACTGT
TGTGTCCGTCATTGGCATCTCTATTCTCACATCTTATATCGATGCTGATAAGGCCA
AAGCACTGAATAATGCAGTGCTTGGCTTATTTAAAGAAGCCGCTGCCAAGGAGG
CGGCCGCTAAATCAGATACAATGATAGTAGTTGCTACTCCGACCCCGGGTTTTAG
CTACGCGAGCGGCTTGACGTACGGCGGTGGTGCGTTTGCGGGTGCGCCTGCGAA
CGGTCCGTCGGAAGGCCAAATCTTTTTCCAGACCGTTCTGCCGGCGTATCAATCC
CCGAATCTGTGTATTGGTCAACTGGCTTGGATGACCGATTACATTAATAAGAACG
GGGTGGGTAACCCAAAAACCTGGGAAGTTATTTCCCAAAACGTTCTGATTTTCTG
CAGCGCGGATACTGCCCTGGTGCTTAATCCGCGCATCGCGGTGTACGACGGCTTC
CACAAAACTAAATGGGCGCCAGCCAAGTTCAATTTTAAAACCCAGAGCCAGGAG
AAATTCTCTGGCAACGTGACCACCCCGATTGCTGCCTTCGGCCATTATCTGTGGG
GTGAGGGCAAGCCGCGTACCGTCGACTTGTCTAGCGTTGGTCTGAAGATCCAGGC
AAACCAGATCGACCCGGTTATGATTGCGGTTAAAAACAACGCAGCAGGCACCTA
CCAGATCTCCGGCAACTTCAACCGTAATACCTTTATCGACGGCGATATTCCGGGT
TTATATCTGGGTAACATTACCATGAAAACGGAAGGCACACTCAAGATCGACGCC
AAGGGTAATTGGAATTATAACGGTGTCGTGCGCGCGTTTAACGATACCTACGACG
CTAATCCGTCGACCCACCGTAGCAAAAGCGCAGAAGATCTGACGACGTTGCTGC
GTTTGACGCAAGGTACGCCGTACGAGATCAGAATCCCGGGTGAGCTGAAGGTGA
GCGGTAGCGGCAAGAAGTAA
32. P864 (P764 FL-FL-P801FL)
Amino acid sequence of P864 (SEQ ID NO: 63)
MPEETLTVVGGGNNSCNVSWGGGNGNNGGAGYSGKYGGTSYEGATSMLKLNDRV
LIQLYLCNPLNPDYIGAPWGSDKDAESIIRANRDKAGKFKANIQNWKTSGTGSLGSP
VVGKSYSSGDVDTYSVSFGKEKYNVLYNRKKDSFTTAYVDGGANKPEHSMKDQAI
AVVKLYLLNESQASVIDTTSGIITDSGKTLSGKLGDKYNTLAKEAADNIKNFQGKKL
RSFNDAMASINELANNPKMKLSQADKTVVSNALKQMDLSALADRFKGLEKAFTWG
DRLLKAEKIREGVVTGITTGDWQKLAFEVEAMYLSGVAGAVALGITTAMISTVAVA
LSLPAVAVSALTVVSVIGISILTSYIDADKAKALNNAVLGLFKGSGSASGSDTMIVVA
TPTPGFSYASGLTYGGGAFAGAPANGPSEGQIFFQTVLPAYQSPNLCIGQLAWMTDYI
NKNGVGNPKTWEVISQNVLIFCSADTALVLNPRIAVYDGFHKTKWAPAKFNFKTQS
QEKFSGNVTTPIAAFGHYLWGEGKPRTVDLSSVGLKIQANQIDPVMIAVKNNAAGTY
QISGNFNRNTFIDGDIPGLYLGNITMKTEGTLKIDAKGNWNYNGVVRAFNDTYDANP
STHRSKSAEDLTTLLRLTQGTPYEIRIPGELKVSGSGKK
P864 domain boundaries:
    1-377: P764 full length
378-384: Flexible linker
385-659: P801 full length
Number of amino acids: 659
Molecular weight: 70068
Theoretical pI: 9.01
DNA Sequence of P864 (SEQ ID NO: 64)
ATGCCTGAAGAAACATTGACTGTCGTAGGTGGTGGTAATAACAGCTGTAATGTTA
GCTGGGGTGGTGGCAATGGTAACAACGGTGGTGCTGGCTATTCTGGTAAATACG
GTGGTACTAGCTATGAAGGTGCAACTAGTATGTTGAAGTTGAATGACCGTGTTTT
AATTCAGCTTTATCTTTGCAATCCGCTTAACCCAGATTATATTGGAGCACCTTGGG
GCAGTGATAAAGATGCAGAATCAATTATCAGAGCTAACAGAGATAAAGCAGGAA
AGTTCAAGGCTAATATTCAGAACTGGAAAACAAGCGGTACAGGTTCTTTAGGTA
GCCCTGTAGTTGGGAAATCTTATAGCTCAGGTGATGTAGATACTTATTCGGTTAG
CTTCGGAAAAGAAAAATACAATGTCCTGTATAACCGTAAAAAAGACTCTTTTACT
ACAGCTTATGTCGATGGCGGCGCAAATAAACCTGAGCATAGCATGAAGGATCAG
GCTATTGCCGTTGTTAAACTTTACCTTCTCAACGAAAGTCAGGCATCGGTAATCG
ATACTACATCGGGAATTATTACTGACTCTGGTAAAACTCTTAGCGGGAAATTAGG
TGATAAATACAACACTCTGGCGAAAGAAGCTGCTGACAATATAAAAAACTTCCA
GGGTAAGAAACTCCGCAGTTTTAATGATGCTATGGCATCTATTAATGAACTAGCT
AACAATCCAAAGATGAAGTTAAGTCAGGCGGATAAAACAGTCGTTTCTAATGCC
CTCAAACAAATGGATTTGTCAGCACTAGCTGACCGATTCAAAGGGTTAGAGAAA
GCCTTTACTTGGGGTGATCGACTTCTTAAAGCCGAGAAAATCAGAGAAGGTGTTG
TTACTGGTATTACCACAGGGGACTGGCAAAAGCTGGCGTTTGAGGTTGAAGCTAT
GTACCTCAGTGGTGTTGCTGGCGCCGTAGCGTTAGGGATTACTACTGCCATGATT
AGCACAGTCGCAGTCGCTTTGTCACTTCCAGCTGTAGCTGTCTCTGCGCTTACTGT
TGTGTCCGTCATTGGCATCTCTATTCTCACATCTTATATCGATGCTGATAAGGCCA
AAGCACTGAATAATGCAGTGCTTGGCTTATTTAAAGGCTCCGGATCGGCTTCTGG
GTCAGATACAATGATAGTAGTTGCTACTCCGACCCCGGGTTTTAGCTACGCGAGC
GGCTTGACGTACGGCGGTGGTGCGTTTGCGGGTGCGCCTGCGAACGGTCCGTCGG
AAGGCCAAATCTTTTTCCAGACCGTTCTGCCGGCGTATCAATCCCCGAATCTGTG
TATTGGTCAACTGGCTTGGATGACCGATTACATTAATAAGAACGGGGTGGGTAAC
CCAAAAACCTGGGAAGTTATTTCCCAAAACGTTCTGATTTTCTGCAGCGCGGATA
CTGCCCTGGTGCTTAATCCGCGCATCGCGGTGTACGACGGCTTCCACAAAACTAA
ATGGGCGCCAGCCAAGTTCAATTTTAAAACCCAGAGCCAGGAGAAATTCTCTGG
CAACGTGACCACCCCGATTGCTGCCTTCGGCCATTATCTGTGGGGTGAGGGCAAG
CCGCGTACCGTCGACTTGTCTAGCGTTGGTCTGAAGATCCAGGCAAACCAGATCG
ACCCGGTTATGATTGCGGTTAAAAACAACGCAGCAGGCACCTACCAGATCTCCG
GCAACTTCAACCGTAATACCTTTATCGACGGCGATATTCCGGGTTTATATCTGGG
TAACATTACCATGAAAACGGAAGGCACACTCAAGATCGACGCCAAGGGTAATTG
GAATTATAACGGTGTCGTGCGCGCGTTTAACGATACCTACGACGCTAATCCGTCG
ACCCACCGTAGCAAAAGCGCAGAAGATCTGACGACGTTGCTGCGTTTGACGCAA
GGTACGCCGTACGAGATCAGAATCCCGGGTGAGCTGAAGGTGAGCGGTAGCGGC
AAGAAGTAA
33. P870 (P764FL-4XRigid Linker-P801FL)
Amino acid sequence of P870 (SEQ ID NO: 65)
MPEETLTVVGGGNNSCNVSWGGGNGNNGGAGYSGKYGGTSYEGATSMLKLNDRV
LIQLYLCNPLNPDYIGAPWGSDKDAESIIRANRDKAGKFKANIQNWKTSGTGSLGSP
VVGKSYSSGDVDTYSVSFGKEKYNVLYNRKKDSFTTAYVDGGANKPEHSMKDQAI
AVVKLYLLNESQASVIDTTSGIITDSGKTLSGKLGDKYNTLAKEAADNIKNFQGKKL
RSFNDAMASINELANNPKMKLSQADKTVVSNALKQMDLSALADRFKGLEKAFTWG
DRLLKAEKIREGVVTGITTGDWQKLAFEVEAMYLSGVAGAVALGITTAMISTVAVA
LSLPAVAVSALTVVSVIGISILTSYIDADKAKALNNAVLGLFKEAAAKEAAAKEAAA
KEAAAKSDTMIVVATPTPGFSYASGLTYGGGAFAGAPANGPSEGQIFFQTVLPAYQS
PNLCIGQLAWMTDYINKNGVGNPKTWEVISQNVLIFCSADTALVLNPRIAVYDGFHK
TKWAPAKFNFKTQSQEKFSGNVTTPIAAFGHYLWGEGKPRTVDLSSVGLKIQANQID
PVMIAVKNNAAGTYQISGNFNRNTFIDGDIPGLYLGNITMKTEGTLKIDAKGNWNYN
GVVRAFNDTYDANPSTHRSKSAEDLTTLLRLTQGTPYEIRIPGELKVSGSGKK
P870 domain boundaries:
    1-377: P764 Full length
378-397: 4X Rigid linker
398-672: P801 full length
Number of amino acids: 672
Molecular weight: 71446
Theoretical pI: 8.98
DNA Sequence of P870 (SEQ ID NO: 66)
ATGCCTGAAGAAACATTGACTGTCGTAGGTGGTGGTAATAACAGCTGTAATGTTA
GCTGGGGTGGTGGCAATGGTAACAACGGTGGTGCTGGCTATTCTGGTAAATACG
GTGGTACTAGCTATGAAGGTGCAACTAGTATGTTGAAGTTGAATGACCGTGTTTT
AATTCAGCTTTATCTTTGCAATCCGCTTAACCCAGATTATATTGGAGCACCTTGGG
GCAGTGATAAAGATGCAGAATCAATTATCAGAGCTAACAGAGATAAAGCAGGAA
AGTTCAAGGCTAATATTCAGAACTGGAAAACAAGCGGTACAGGTTCTTTAGGTA
GCCCTGTAGTTGGGAAATCTTATAGCTCAGGTGATGTAGATACTTATTCGGTTAG
CTTCGGAAAAGAAAAATACAATGTCCTGTATAACCGTAAAAAAGACTCTTTTACT
ACAGCTTATGTCGATGGCGGCGCAAATAAACCTGAGCATAGCATGAAGGATCAG
GCTATTGCCGTTGTTAAACTTTACCTTCTCAACGAAAGTCAGGCATCGGTAATCG
ATACTACATCGGGAATTATTACTGACTCTGGTAAAACTCTTAGCGGGAAATTAGG
TGATAAATACAACACTCTGGCGAAAGAAGCTGCTGACAATATAAAAAACTTCCA
GGGTAAGAAACTCCGCAGTTTTAATGATGCTATGGCATCTATTAATGAACTAGCT
AACAATCCAAAGATGAAGTTAAGTCAGGCGGATAAAACAGTCGTTTCTAATGCC
CTCAAACAAATGGATTTGTCAGCACTAGCTGACCGATTCAAAGGGTTAGAGAAA
GCCTTTACTTGGGGTGATCGACTTCTTAAAGCCGAGAAAATCAGAGAAGGTGTTG
TTACTGGTATTACCACAGGGGACTGGCAAAAGCTGGCGTTTGAGGTTGAAGCTAT
GTACCTCAGTGGTGTTGCTGGCGCCGTAGCGTTAGGGATTACTACTGCCATGATT
AGCACAGTCGCAGTCGCTTTGTCACTTCCAGCTGTAGCTGTCTCTGCGCTTACTGT
TGTGTCCGTCATTGGCATCTCTATTCTCACATCTTATATCGATGCTGATAAGGCCA
AAGCACTGAATAATGCAGTGCTTGGCTTATTTAAAGAAGCCGCTGCCAAGGAGG
CGGCCGCTAAAGAAGCCGCTGCCAAGGAGGCGGCCGCTAAATCAGATACAATGA
TAGTAGTTGCTACTCCGACCCCGGGTTTTAGCTACGCGAGCGGCTTGACGTACGG
CGGTGGTGCGTTTGCGGGTGCGCCTGCGAACGGTCCGTCGGAAGGCCAAATCTTT
TTCCAGACCGTTCTGCCGGCGTATCAATCCCCGAATCTGTGTATTGGTCAACTGG
CTTGGATGACCGATTACATTAATAAGAACGGGGTGGGTAACCCAAAAACCTGGG
AAGTTATTTCCCAAAACGTTCTGATTTTCTGCAGCGCGGATACTGCCCTGGTGCTT
AATCCGCGCATCGCGGTGTACGACGGCTTCCACAAAACTAAATGGGCGCCAGCC
AAGTTCAATTTTAAAACCCAGAGCCAGGAGAAATTCTCTGGCAACGTGACCACC
CCGATTGCTGCCTTCGGCCATTATCTGTGGGGTGAGGGCAAGCCGCGTACCGTCG
ACTTGTCTAGCGTTGGTCTGAAGATCCAGGCAAACCAGATCGACCCGGTTATGAT
TGCGGTTAAAAACAACGCAGCAGGCACCTACCAGATCTCCGGCAACTTCAACCG
TAATACCTTTATCGACGGCGATATTCCGGGTTTATATCTGGGTAACATTACCATG
AAAACGGAAGGCACACTCAAGATCGACGCCAAGGGTAATTGGAATTATAACGGT
GTCGTGCGCGCGTTTAACGATACCTACGACGCTAATCCGTCGACCCACCGTAGCA
AAAGCGCAGAAGATCTGACGACGTTGCTGCGTTTGACGCAAGGTACGCCGTACG
AGATCAGAATCCCGGGTGAGCTGAAGGTGAGCGGTAGCGGCAAGAAGTAA
34. P867: 1-53 amino acids of P849 (P801 homolog from K. quasipneumoniae)
replaced with 1-22 amino acids of P801
Amino acid sequence of P867 (SEQ ID NO: 67)
MSDTMIVVATPTPGFSYASGLTYGGGMFSPTISGPSEGQIFLQVTLPYYQSEKFCADQ
LNWLVQYIKKHGASDPMTIQVAANNIRYLCNADTNLVKNPKTLVYDAFHSRSPAPA
NYDYRSMNLKQMSGSVVTPGAAFAHYLWGNGETRFVNLPDVGLKVTPQQIPELMK
LVNSGVTGAIPVSVKFSRDTSTDSITAGAYLGHITLQTEGTLNVTGNGAWTYNGVIR
AYDDTYDFNLGNFRGPIAESLTFLGSTFSGKPYQISLPGQINISGSGKK
P867 domain boundaries:
    1-22: 22 amino acids from P801 translocation domain
23-275: P849
Number of amino acids: 275
Molecular weight: 29701
Theoretical pI: 7.72
DNA Sequence of P867 (SEQ ID NO: 68)
ATGTCAGATACAATGATAGTAGTTGCTACTCCGACCCCGGGTTTTAGCTACGCGA
GCGGCTTGACGTACGGTGGCGGTATGTTTAGCCCGACCATCAGCGGTCCGAGCG
AGGGTCAGATTTTTCTGCAAGTGACCCTGCCGTACTATCAGAGCGAAAAGTTCTG
CGCGGATCAGCTGAACTGGCTGGTGCAATATATTAAGAAACACGGCGCGAGCGA
CCCGATGACCATCCAAGTTGCGGCGAACAACATTCGTTACCTGTGCAACGCGGAC
ACCAACCTGGTGAAGAACCCGAAAACCCTGGTTTATGATGCGTTCCACAGCCGTA
GCCCGGCGCCGGCGAACTACGACTATCGTAGCATGAACCTGAAGCAGATGAGCG
GCAGCGTGGTTACGCCGGGTGCGGCGTTTGCGCACTACCTGTGGGGCAACGGCG
AGACCCGTTTTGTTAACCTGCCGGATGTGGGTCTGAAGGTTACCCCGCAGCAAAT
CCCGGAACTGATGAAACTGGTGAACAGCGGCGTTACCGGTGCGATTCCGGTGAG
CGTTAAATTCAGCCGTGACACCAGCACCGATAGCATCACCGCGGGCGCGTACCT
GGGTCACATTACCCTGCAAACCGAGGGCACCCTGAACGTGACCGGCAACGGTGC
GTGGACCTATAACGGTGTTATCCGTGCGTACGACGATACCTATGACTTCAACCTG
GGCAACTTTCGTGGTCCGATTGCGGAAAGCCTGACCTTCCTGGGCAGCACCTTTA
GCGGCAAGCCGTACCAGATCAGCCTGCCGGGTCAAATCAACATTAGCGGCAGCG
GTAAAAAGTAA
35. P875 (P801-Rigid Linker-GP36)
Amino acid sequence of P875 (SEQ ID NO: 69)
MSDTMIVVATPTPGFSYASGLTYGGGAFAGAPANGPSEGQIFFQTVLPAYQSPNLCIG
QLAWMTDYINKNGVGNPKTWEVISQNVLIFCSADTALVLNPRIAVYDGFHKTKWAP
AKFNFKTQSQEKFSGNVTTPIAAFGHYLWGEGKPRTVDLSSVGLKIQANQIDPVMIA
VKNNAAGTYQISGNFNRNTFIDGDIPGLYLGNITMKTEGTLKIDAKGNWNYNGVVR
AFNDTYDANPSTHRSKSAEDLTTLLRLTQGTPYEIRIPGELKVSGSGKKEAAAKEAA
AKGVALDRTRVDPQAVGNEVLKRNADKLNAMRGAEYGANVKVSGTDIRMNGGNS
AGMLKQDVFNWRKELAQFEAYRGEAYKDADGYSVGLGHYLGSGNAGAGTTVTPE
QAAQWFAEDTDRALDQGVRLADELGVTNNASILGLAGMAFQMGEGRARQFRNTFQ
AIKDRNKEAFEAGVRNSKWYTQTPNRAEAFIKRMAPHFDTPSQIGVDWYSAATAE
P875 domain boundaries:
    1-276: P801 Full length
277-286: 2X Rigid linker
287-502: GP36 catalytic domain
Number of amino acids: 502
Molecular weight: 54304
Theoretical pI: 8.53
DNA Sequence of P875 (SEQ ID NO: 70)
ATGTCAGATACAATGATAGTAGTTGCTACTCCGACCCCGGGTTTTAGCTACGCGA
GCGGCTTGACGTACGGCGGTGGTGCGTTTGCGGGTGCGCCTGCGAACGGTCCGTC
GGAAGGCCAAATCTTTTTCCAGACCGTTCTGCCGGCGTATCAATCCCCGAATCTG
TGTATTGGTCAACTGGCTTGGATGACCGATTACATTAATAAGAACGGGGTGGGTA
ACCCAAAAACCTGGGAAGTTATTTCCCAAAACGTTCTGATTTTCTGCAGCGCGGA
TACTGCCCTGGTGCTTAATCCGCGCATCGCGGTGTACGACGGCTTCCACAAAACT
AAATGGGCGCCAGCCAAGTTCAATTTTAAAACCCAGAGCCAGGAGAAATTCTCT
GGCAACGTGACCACCCCGATTGCTGCCTTCGGCCATTATCTGTGGGGTGAGGGCA
AGCCGCGTACCGTCGACTTGTCTAGCGTTGGTCTGAAGATCCAGGCAAACCAGAT
CGACCCGGTTATGATTGCGGTTAAAAACAACGCAGCAGGCACCTACCAGATCTC
CGGCAACTTCAACCGTAATACCTTTATCGACGGCGATATTCCGGGTTTATATCTG
GGTAACATTACCATGAAAACGGAAGGCACACTCAAGATCGACGCCAAGGGTAAT
TGGAATTATAACGGTGTCGTGCGCGCGTTTAACGATACCTACGACGCTAATCCGT
CGACCCACCGTAGCAAAAGCGCAGAAGATCTGACGACGTTGCTGCGTTTGACGC
AAGGTACGCCGTACGAGATCAGAATCCCGGGTGAGCTGAAGGTGAGCGGTAGCG
GCAAGAAGGAAGCCGCTGCCAAGGAGGCGGCCGCTAAAGGTGTGGCCCTGGACC
GCACGCGGGTTGATCCCCAGGCAGTCGGCAACGAGGTGCTCAAGCGCAACGCGG
ATAAGCTGAATGCGATGCGGGGCGCCGAGTACGGTGCCAACGTCAAGGTCAGCG
GCACGGACATTCGCATGAACGGGGGTAACAGTGCCGGCATGCTGAAGCAGGACG
TGTTCAACTGGCGGAAGGAACTGGCTCAGTTCGAGGCTTACCGAGGGGAGGCGT
ATAAGGATGCCGATGGTTATAGTGTGGGCCTGGGGCATTACCTGGGCAGTGGCA
ATGCTGGGGCAGGTACTACAGTCACGCCTGAGCAAGCCGCGCAGTGGTTCGCCG
AGGACACCGACCGCGCACTCGACCAGGGTGTGAGGTTGGCCGACGAGCTGGGCG
TTACGAACAATGCCTCTATCCTGGGATTGGCCGGTATGGCCTTCCAGATGGGCGA
AGGACGTGCCCGGCAGTTCCGTAACACCTTCCAGGCGATCAAGGATCGCAACAA
GGAAGCCTTCGAGGCTGGTGTGCGAAACAGCAAGTGGTACACGCAGACGCCCAA
CCGGGCCGAGGCATTCATCAAGCGCATGGCGCCCCACTTCGATACACCGAGTCA
AATCGGTGTCGATTGGTACAGCGCCGCAACAGCGGAGTGA
36. P889 (P764-GP36CD)
Amino acid sequence of P889 (SEQ ID NO: 71)
MPEETLTVVGGGNNSCNVSWGGGNGNNGGAGYSGKYGGTSYEGATSMLKLNDRV
LIQLYLCNPLNPDYIGAPWGSDKDAESIIRANRDKAGKFKANIONWKTSGTGSLGSP
VVGKSYSSGDVDTYSVSFGKEKYNVLYNRKKDSFTTAYVDGGANKPEHSMKDQAI
AVVKLYLLNESQASVIDTTSGIITDSGKTLSGKLGDKYNTLAKEAADNIKNFQGKKL
RSFNDAMASINELANNPKMKLSQADKTVVSNALKQMDLSALADRFKGLEKAFTWG
DRLLKAEKIREGVVTGITTGDWQKLAFEVEAMYLSGVAGAVALGITTAMISTVAVA
LSLPAVAVSALTVVSVIGISILTSYIDADKAKALNNAVLGLFKGVALDRTRVDPQAV
GNEVLKRNADKLNAMRGAEYGANVKVSGTDIRMNGGNSAGMLKQDVENWRKEL
AQFEAYRGEAYKDADGYSVGLGHYLGSGNAGAGTTVTPEQAAQWFAEDTDRALD
QGVRLADELGVTNNASILGLAGMAFQMGEGRARQFRNTFQAIKDRNKEAFEAGVR
NSKWYTQTPNRAEAFIKRMAPHFDTPSQIGVDWYSAATAE
P889 domain boundaries:
    1-377: P764 Full length
378-593: GP36 catalytic domain
Number of amino acids: 593
Molecular weight: 63412
Theoretical pI: 8.73
P889 DNA sequence (SEQ ID NO: 72)
ATGCCTGAAGAAACATTGACTGTCGTAGGTGGTGGTAATAACAGCTGTAATGTTA
GCTGGGGTGGTGGCAATGGTAACAACGGTGGTGCTGGCTATTCTGGTAAATACG
GTGGTACTAGCTATGAAGGTGCAACTAGTATGTTGAAGTTGAATGACCGTGTTTT
AATTCAGCTTTATCTTTGCAATCCGCTTAACCCAGATTATATTGGAGCACCTTGGG
GCAGTGATAAAGATGCAGAATCAATTATCAGAGCTAACAGAGATAAAGCAGGAA
AGTTCAAGGCTAATATTCAGAACTGGAAAACAAGCGGTACAGGTTCTTTAGGTA
GCCCTGTAGTTGGGAAATCTTATAGCTCAGGTGATGTAGATACTTATTCGGTTAG
CTTCGGAAAAGAAAAATACAATGTCCTGTATAACCGTAAAAAAGACTCTTTTACT
ACAGCTTATGTCGATGGCGGCGCAAATAAACCTGAGCATAGCATGAAGGATCAG
GCTATTGCCGTTGTTAAACTTTACCTTCTCAACGAAAGTCAGGCATCGGTAATCG
ATACTACATCGGGAATTATTACTGACTCTGGTAAAACTCTTAGCGGGAAATTAGG
TGATAAATACAACACTCTGGCGAAAGAAGCTGCTGACAATATAAAAAACTTCCA
GGGTAAGAAACTCCGCAGTTTTAATGATGCTATGGCATCTATTAATGAACTAGCT
AACAATCCAAAGATGAAGTTAAGTCAGGCGGATAAAACAGTCGTTTCTAATGCC
CTCAAACAAATGGATTTGTCAGCACTAGCTGACCGATTCAAAGGGTTAGAGAAA
GCCTTTACTTGGGGTGATCGACTTCTTAAAGCCGAGAAAATCAGAGAAGGTGTTG
TTACTGGTATTACCACAGGGGACTGGCAAAAGCTGGCGTTTGAGGTTGAAGCTAT
GTACCTCAGTGGTGTTGCTGGCGCCGTAGCGTTAGGGATTACTACTGCCATGATT
AGCACAGTCGCAGTCGCTTTGTCACTTCCAGCTGTAGCTGTCTCTGCGCTTACTGT
TGTGTCCGTCATTGGCATCTCTATTCTCACATCTTATATCGATGCTGATAAGGCCA
AAGCACTGAATAATGCAGTGCTTGGCTTATTTAAAGGTGTGGCCCTGGACCGCAC
GCGGGTTGATCCCCAGGCAGTCGGCAACGAGGTGCTCAAGCGCAACGCGGATAA
GCTGAATGCGATGCGGGGCGCCGAGTACGGTGCCAACGTCAAGGTCAGCGGCAC
GGACATTCGCATGAACGGGGGTAACAGTGCCGGCATGCTGAAGCAGGACGTGTT
CAACTGGCGGAAGGAACTGGCTCAGTTCGAGGCTTACCGAGGGGAGGCGTATAA
GGATGCCGATGGTTATAGTGTGGGCCTGGGGCATTACCTGGGCAGTGGCAATGCT
GGGGCAGGTACTACAGTCACGCCTGAGCAAGCCGCGCAGTGGTTCGCCGAGGAC
ACCGACCGCGCACTCGACCAGGGTGTGAGGTTGGCCGACGAGCTGGGCGTTACG
AACAATGCCTCTATCCTGGGATTGGCCGGTATGGCCTTCCAGATGGGCGAAGGAC
GTGCCCGGCAGTTCCGTAACACCTTCCAGGCGATCAAGGATCGCAACAAGGAAG
CCTTCGAGGCTGGTGTGCGAAACAGCAAGTGGTACACGCAGACGCCCAACCGGG
CCGAGGCATTCATCAAGCGCATGGCGCCCCACTTCGATACACCGAGTCAAATCG
GTGTCGATTGGTACAGCGCCGCAACAGCGGAGTGA
37. P891 (P764-FL-GP36CD)
Amino acid sequence of P891 (SEQ ID NO: 73)
MPEETLTVVGGGNNSCNVSWGGGNGNNGGAGYSGKYGGTSYEGATSMLKLNDRV
LIQLYLCNPLNPDYIGAPWGSDKDAESIIRANRDKAGKFKANIQNWKTSGTGSLGSP
VVGKSYSSGDVDTYSVSFGKEKYNVLYNRKKDSFTTAYVDGGANKPEHSMKDQAI
AVVKLYLLNESQASVIDTTSGIITDSGKTLSGKLGDKYNTLAKEAADNIKNFQGKKL
RSFNDAMASINELANNPKMKLSQADKTVVSNALKQMDLSALADRFKGLEKAFTWG
DRLLKAEKIREGVVTGITTGDWQKLAFEVEAMYLSGVAGAVALGITTAMISTVAVA
LSLPAVAVSALTVVSVIGISILTSYIDADKAKALNNAVLGLFKGSGSASGGVALDRTR
VDPQAVGNEVLKRNADKLNAMRGAEYGANVKVSGTDIRMNGGNSAGMLKQDVEN
WRKELAQFEAYRGEAYKDADGYSVGLGHYLGSGNAGAGTTVTPEQAAQWFAEDT
DRALDQGVRLADELGVINNASILGLAGMAFQMGEGRARQFRNTFQAIKDRNKEAFE
AGVRNSKWYTQTPNRAEAFIKRMAPHFDTPSQIGVDWYSAATAE
P891 domain boundaries:
    1-377: P764 Full length
378-384: Flexible linker
385-600: GP36 catalytic domain
Number of amino acids: 600
Molecular weight: 63915
Theoretical pI: 8.73
DNA Sequence of P891 (SEQ ID NO: 74)
ATGCCTGAAGAAACATTGACTGTCGTAGGTGGTGGTAATAACAGCTGTAATGTTA
GCTGGGGTGGTGGCAATGGTAACAACGGTGGTGCTGGCTATTCTGGTAAATACG
GTGGTACTAGCTATGAAGGTGCAACTAGTATGTTGAAGTTGAATGACCGTGTTTT
AATTCAGCTTTATCTTTGCAATCCGCTTAACCCAGATTATATTGGAGCACCTTGGG
GCAGTGATAAAGATGCAGAATCAATTATCAGAGCTAACAGAGATAAAGCAGGAA
AGTTCAAGGCTAATATTCAGAACTGGAAAACAAGCGGTACAGGTTCTTTAGGTA
GCCCTGTAGTTGGGAAATCTTATAGCTCAGGTGATGTAGATACTTATTCGGTTAG
CTTCGGAAAAGAAAAATACAATGTCCTGTATAACCGTAAAAAAGACTCTTTTACT
ACAGCTTATGTCGATGGCGGCGCAAATAAACCTGAGCATAGCATGAAGGATCAG
GCTATTGCCGTTGTTAAACTTTACCTTCTCAACGAAAGTCAGGCATCGGTAATCG
ATACTACATCGGGAATTATTACTGACTCTGGTAAAACTCTTAGCGGGAAATTAGG
TGATAAATACAACACTCTGGCGAAAGAAGCTGCTGACAATATAAAAAACTTCCA
GGGTAAGAAACTCCGCAGTTTTAATGATGCTATGGCATCTATTAATGAACTAGCT
AACAATCCAAAGATGAAGTTAAGTCAGGCGGATAAAACAGTCGTTTCTAATGCC
CTCAAACAAATGGATTTGTCAGCACTAGCTGACCGATTCAAAGGGTTAGAGAAA
GCCTTTACTTGGGGTGATCGACTTCTTAAAGCCGAGAAAATCAGAGAAGGTGTTG
TTACTGGTATTACCACAGGGGACTGGCAAAAGCTGGCGTTTGAGGTTGAAGCTAT
GTACCTCAGTGGTGTTGCTGGCGCCGTAGCGTTAGGGATTACTACTGCCATGATT
AGCACAGTCGCAGTCGCTTTGTCACTTCCAGCTGTAGCTGTCTCTGCGCTTACTGT
TGTGTCCGTCATTGGCATCTCTATTCTCACATCTTATATCGATGCTGATAAGGCCA
AAGCACTGAATAATGCAGTGCTTGGCTTATTTAAAGGCTCCGGATCGGCTTCTGG
GGGTGTGGCCCTGGACCGCACGCGGGTTGATCCCCAGGCAGTCGGCAACGAGGT
GCTCAAGCGCAACGCGGATAAGCTGAATGCGATGCGGGGCGCCGAGTACGGTGC
CAACGTCAAGGTCAGCGGCACGGACATTCGCATGAACGGGGGTAACAGTGCCGG
CATGCTGAAGCAGGACGTGTTCAACTGGCGGAAGGAACTGGCTCAGTTCGAGGC
TTACCGAGGGGAGGCGTATAAGGATGCCGATGGTTATAGTGTGGGCCTGGGGCA
TTACCTGGGCAGTGGCAATGCTGGGGCAGGTACTACAGTCACGCCTGAGCAAGC
CGCGCAGTGGTTCGCCGAGGACACCGACCGCGCACTCGACCAGGGTGTGAGGTT
GGCCGACGAGCTGGGCGTTACGAACAATGCCTCTATCCTGGGATTGGCCGGTATG
GCCTTCCAGATGGGCGAAGGACGTGCCCGGCAGTTCCGTAACACCTTCCAGGCG
ATCAAGGATCGCAACAAGGAAGCCTTCGAGGCTGGTGTGCGAAACAGCAAGTGG
TACACGCAGACGCCCAACCGGGCCGAGGCATTCATCAAGCGCATGGCGCCCCAC
TTCGATACACCGAGTCAAATCGGTGTCGATTGGTACAGCGCCGCAACAGCGGAG
TGA
38. P892 (P764-RL-GP36CD)
Amino acid sequence of P891 (SEQ ID NO: 75)
MPEETLTVVGGGNNSCNVSWGGGNGNNGGAGYSGKYGGTSYEGATSMLKLNDRV
LIQLYLCNPLNPDYIGAPWGSDKDAESIIRANRDKAGKFKANIQNWKTSGTGSLGSP
VVGKSYSSGDVDTYSVSFGKEKYNVLYNRKKDSFTTAYVDGGANKPEHSMKDQAI
AVVKLYLLNESQASVIDTTSGIITDSGKTLSGKLGDKYNTLAKEAADNIKNFQGKKL
RSFNDAMASINELANNPKMKLSQADKTVVSNALKQMDLSALADRFKGLEKAFTWG
DRLLKAEKIREGVVTGITTGDWQKLAFEVEAMYLSGVAGAVALGITTAMISTVAVA
LSLPAVAVSALTVVSVIGISILTSYIDADKAKALNNAVLGLFKEAAAKEAAAKGVAL
DRTRVDPQAVGNEVLKRNADKLNAMRGAEYGANVKVSGTDIRMNGGNSAGMLKQ
DVENWRKELAQFEAYRGEAYKDADGYSVGLGHYLGSGNAGAGTTVTPEQAAQWF
AEDTDRALDQGVRLADELGVINNASILGLAGMAFQMGEGRARQFRNTFQAIKDRN
KEAFEAGVRNSKWYTQTPNRAEAFIKRMAPHFDTPSQIGVDWYSAATAE
P892 domain boundaries:
    1-377: P764 TDRD
378-387: Rigid linker
388-603: GP36 CD
Number of amino acids: 603
Molecular weight: 64353
Theoretical pI: 8.72
DNA Sequence of P892 (SEQ ID NO: 76)
ATGCCTGAAGAAACATTGACTGTCGTAGGTGGTGGTAATAACAGCTGTAATGTTA
GCTGGGGTGGTGGCAATGGTAACAACGGTGGTGCTGGCTATTCTGGTAAATACG
GTGGTACTAGCTATGAAGGTGCAACTAGTATGTTGAAGTTGAATGACCGTGTTTT
AATTCAGCTTTATCTTTGCAATCCGCTTAACCCAGATTATATTGGAGCACCTTGGG
GCAGTGATAAAGATGCAGAATCAATTATCAGAGCTAACAGAGATAAAGCAGGAA
AGTTCAAGGCTAATATTCAGAACTGGAAAACAAGCGGTACAGGTTCTTTAGGTA
GCCCTGTAGTTGGGAAATCTTATAGCTCAGGTGATGTAGATACTTATTCGGTTAG
CTTCGGAAAAGAAAAATACAATGTCCTGTATAACCGTAAAAAAGACTCTTTTACT
ACAGCTTATGTCGATGGCGGCGCAAATAAACCTGAGCATAGCATGAAGGATCAG
GCTATTGCCGTTGTTAAACTTTACCTTCTCAACGAAAGTCAGGCATCGGTAATCG
ATACTACATCGGGAATTATTACTGACTCTGGTAAAACTCTTAGCGGGAAATTAGG
TGATAAATACAACACTCTGGCGAAAGAAGCTGCTGACAATATAAAAAACTTCCA
GGGTAAGAAACTCCGCAGTTTTAATGATGCTATGGCATCTATTAATGAACTAGCT
AACAATCCAAAGATGAAGTTAAGTCAGGCGGATAAAACAGTCGTTTCTAATGCC
CTCAAACAAATGGATTTGTCAGCACTAGCTGACCGATTCAAAGGGTTAGAGAAA
GCCTTTACTTGGGGTGATCGACTTCTTAAAGCCGAGAAAATCAGAGAAGGTGTTG
TTACTGGTATTACCACAGGGGACTGGCAAAAGCTGGCGTTTGAGGTTGAAGCTAT
GTACCTCAGTGGTGTTGCTGGCGCCGTAGCGTTAGGGATTACTACTGCCATGATT
AGCACAGTCGCAGTCGCTTTGTCACTTCCAGCTGTAGCTGTCTCTGCGCTTACTGT
TGTGTCCGTCATTGGCATCTCTATTCTCACATCTTATATCGATGCTGATAAGGCCA
AAGCACTGAATAATGCAGTGCTTGGCTTATTTAAAGAAGCCGCTGCCAAGGAGG
CGGCCGCTAAAGGTGTGGCCCTGGACCGCACGCGGGTTGATCCCCAGGCAGTCG
GCAACGAGGTGCTCAAGCGCAACGCGGATAAGCTGAATGCGATGCGGGGCGCCG
AGTACGGTGCCAACGTCAAGGTCAGCGGCACGGACATTCGCATGAACGGGGGTA
ACAGTGCCGGCATGCTGAAGCAGGACGTGTTCAACTGGCGGAAGGAACTGGCTC
AGTTCGAGGCTTACCGAGGGGAGGCGTATAAGGATGCCGATGGTTATAGTGTGG
GCCTGGGGCATTACCTGGGCAGTGGCAATGCTGGGGCAGGTACTACAGTCACGC
CTGAGCAAGCCGCGCAGTGGTTCGCCGAGGACACCGACCGCGCACTCGACCAGG
GTGTGAGGTTGGCCGACGAGCTGGGCGTTACGAACAATGCCTCTATCCTGGGATT
GGCCGGTATGGCCTTCCAGATGGGCGAAGGACGTGCCCGGCAGTTCCGTAACAC
CTTCCAGGCGATCAAGGATCGCAACAAGGAAGCCTTCGAGGCTGGTGTGCGAAA
CAGCAAGTGGTACACGCAGACGCCCAACCGGGCCGAGGCATTCATCAAGCGCAT
GGCGCCCCACTTCGATACACCGAGTCAAATCGGTGTCGATTGGTACAGCGCCGCA
ACAGCGGAGTGA
```

1. A polypeptide comprising one or more translocation domains, one or more receptor-binding domains, and a killing domain, wherein each of the domains is derived from a naturally-occurring klebicin, and wherein the killing domain and at least one of the receptor-binding domains are derived from two different naturally-occurring klebicins.

2. The polypeptide of claim 1, comprising, from the N-terminus to the C-terminus, the translocation domain(s), the receptor-binding domain(s), and the killing domain.

3. The polypeptide of claim 1, comprising two receptor-binding domains derived from two different naturally-occurring klebicins.

4. The polypeptide of claim 2, further comprising a peptide linker between the receptor-binding domain(s) and the killing domain.

5. The polypeptide of claim 1, wherein the killing domain comprises a full-length naturally-occurring klebicin.

6. The polypeptide of claim 1, wherein one of the translocation domain and one of the receptor-binding domains are derived from P628 or P764.

7. The polypeptide of claim 1, wherein the killing domain comprises full length P764 or P801.

8. The polypeptide of claim 3, wherein the two receptor-binding domains are derived from P764 and P774.

9. The polypeptide of claim 1, comprising the amino acid sequence of SEQ ID NO:7, 33, 45, 47, 51, 57, 59, 61, 65, 67, 69, 71, 73, or 75.

10. The polypeptide of claim 1, consisting of the amino acid sequence of SEQ ID NO:7, 33, 45, or 47.

11. A nucleic acid comprising a polynucleotide sequence encoding the polypeptide of claim 1.

12. An expression cassette comprising the nucleic acid of claim 11.

13. A vector comprising the expression cassette of claim 12.

14. A host cell comprising the expression cassette of claim 12.

15. A method of recombinantly producing the polypeptide of any claim 1, comprising culturing a host cell comprising an expression cassette, wherein the expression cassette comprises a polynucleotide sequence encoding the polypeptide under conditions permitting expression of the polypeptide.

16. A composition comprising the polypeptide of claim 1, and a physiologically acceptable excipient.

17. The composition of claim 16, formulated for administration by injection or inhalation.

18. A method for suppressing growth of Klebsiella pneumoniae, comprising applying an effective amount of the composition of claim 16 to a location where Klebsiella pneumoniae is present.

19. The method of claim 18, wherein the composition is applied to a patient suffering from a Klebsiella pneumoniae infection by injection.

20. The method of claim 18, wherein the composition is applied to a patient suffering from a Klebsiella pneumoniae infection by inhalation.

21-24. (canceled)

25. A kit for suppression of growth of Klebsiella pneumoniae, comprising a first container containing a composition comprising an effective amount of the polypeptide of claim 1.

26. The kit of claim 25, wherein the kit is formulated for administration of the polypeptide by injection.

27. The kit of claim 25, wherein the kit is formulated for administration of the polypeptide by inhalation.

28. (canceled)


SEQUENCE LISTING (77 sequences)

SEQ ID NO: 1 - protein, 808 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLEVSMGLKLNLTWFDKKTEEFKGGEYSKDFGDDGSVIESLGMPLKDNINNGWFDVEKSWVSILQPHFKNVIDISKFDYFVSFDYRDGNW

SEQ ID NO: 2 - DNA, 2427 bp, synthetic construct
  source 1..2427: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatctttaagaggtaagtatgggacttaaattaaatttaacctggtttgataagaaaaccgaagagtttaaaggtggtgaatactcaaaagacttcggtgatgatggttctgtcattgaaagtctggggatgcctttaaaggataatattaataatggttggtttgatgttgaaaaatcatgggtttcgatattacagccacactttaaaaatgtaatcgatattagtaaatttgattactttgtatcatttgattatcgggatggtaactggtaa

SEQ ID NO: 3 - protein, 815 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSGSGSASGSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLEVSMGLKLNLTWFDKKTEEFKGGEYSKDFGDDGSVIESLGMPLKDNINNGWFDVEKSWVSILQPHFKNVIDISKFDYFVSFDYRDGNW

SEQ ID NO: 4 - DNA, 2448 bp, synthetic construct
  source 1..2448: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatggctccggatcggcttctgggagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatctttaagaggtaagtatgggacttaaattaaatttaacctggtttgataagaaaaccgaagagtttaaaggtggtgaatactcaaaagacttcggtgatgatggttctgtcattgaaagtctggggatgcctttaaaggataatattaataatggttggtttgatgttgaaaaatcatgggtttcgatattacagccacactttaaaaatgtaatcgatattagtaaatttgattactttgtatcatttgattatcgggatggtaactggtaa

SEQ ID NO: 5 - protein, 818 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSEAAAKEAAAKSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLEVSMGLKLNLTWFDKKTEEFKGGEYSKDFGDDGSVIESLGMPLKDNINNGWFDVEKSWVSILQPHFKNVIDISKFDYFVSFDYRDGNW

SEQ ID NO: 6 - DNA, 2457 bp, synthetic construct
  source 1..2457: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatgaagccgctgccaaggaggcggccgctaaaagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatctttaagaggtaagtatgggacttaaattaaatttaacctggtttgataagaaaaccgaagagtttaaaggtggtgaatactcaaaagacttcggtgatgatggttctgtcattgaaagtctggggatgcctttaaaggataatattaataatggttggtttgatgttgaaaaatcatgggtttcgatattacagccacactttaaaaatgtaatcgatattagtaaatttgattactttgtatcatttgattatcgggatggtaactggtaa

SEQ ID NO: 7 - protein, 846 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKEAAAKEAAAKPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFK

SEQ ID NO: 8 - DNA, 2541 bp, synthetic construct
  source 1..2541: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaaggaagccgctgccaaggaggcggccgctaaacctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaataa

SEQ ID NO: 9 - protein, 843 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKGSGSASGPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFK

SEQ ID NO: 10 - DNA, 2532 bp, synthetic construct
  source 1..2532: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagggctccggatcggcttctgggcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaataa

SEQ ID NO: 11 - protein, 785 aa, synthetic construct
MSETMVVVAPPTGFEPAGYGGGLFSPSTPNNSPSQGQIFLQVTLPYYQSAKFCQDSMAWLAQYVKTHGATDPLTIQVVANNIRYFLNADTNLCHNPRQNVWEAFHSEMTGSGPAPAKYDYKSMSLKQEAAAKEAAAKSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLEVSMGLKLNLTWFDKKTEEFKGGEYSKDFGDDGSVIESLGMPLKDNINNGWFDVEKSWVSILQPHFKNVIDISKFDYFVSFDYRDGNW

SEQ ID NO: 12 - DNA, 2361 bp, synthetic construct
  source 1..2361: mol_type = other DNA
atgagcgagaccatggttgttgttgcgccgccgaccggttttgagccggcgggttacggtggtggcctgttcagcccgagcaccccgaacaacagcccgagccagggtcaaatcttcctgcaagtgaccctgccgtactatcagagcgcgaagttttgccaagacagcatggcgtggctggcgcagtacgttaaaacccacggcgcgaccgatccgctgaccatccaagtggttgcgaacaacattcgttacttcctgaacgcggacaccaacctgtgccacaacccgcgtcagaacgtgtgggaggcgtttcacagcgaaatgaccggtagcggtccggcgccggcgaagtacgattataaaagcatgagcctgaagcaagaagccgctgccaaggaggcggccgctaaaagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatctttaagaggtaagtatgggacttaaattaaatttaacctggtttgataagaaaaccgaagagtttaaaggtggtgaatactcaaaagacttcggtgatgatggttctgtcattgaaagtctggggatgcctttaaaggataatattaataatggttggtttgatgttgaaaaatcatgggtttcgatattacagccacactttaaaaatgtaatcgatattagtaaatttgattactttgtatcatttgattatcgggatggtaactggtaa

SEQ ID NO: 13 - protein, 744 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKGSGSASGSETMVVVAPPTGFEPAGYGGGLFSPSTPNNSPSQGQIFLQVTLPYYQSAKFCQDSMAWLAQYVKTHGATDPLTIQVVANNIRYFLNADTNLCHNPRQNVWEAFHSEMTGSGPAPAKYDYKSMSLKQMSGNVVTPAAAFGHYLWGNGEERYVNLPDVGLKITPQQIPELMNIVNSGVTGNIPVDLNFNHNTYDSGGVIPASYLGNVSLRTIGSLNIQNSGEWTYNGVVRAYNDYYDFNLGDYRGAIAESLTYLGAQFSGKPYHIAMPGEINISGAGHR

SEQ ID NO: 14 - DNA, 2235 bp, synthetic construct
  source 1..2235: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagggctccggatcggcttctgggagcgagaccatggttgttgttgcgccgccgaccggttttgagccggcgggttacggtggtggcctgttcagcccgagcaccccgaacaacagcccgagccagggtcaaatcttcctgcaagtgaccctgccgtactatcagagcgcgaagttttgccaagacagcatggcgtggctggcgcagtacgttaaaacccacggcgcgaccgatccgctgaccatccaagtggttgcgaacaacattcgttacttcctgaacgcggacaccaacctgtgccacaacccgcgtcagaacgtgtgggaggcgtttcacagcgaaatgaccggtagcggtccggcgccggcgaagtacgattataaaagcatgagcctgaagcaaatgagcggtaacgtggttaccccggcggcggcgtttggtcactacctgtggggtaacggcgaggaacgttatgtgaacctgccggacgttggtctgaaaatcaccccgcagcaaattccggagctgatgaacatcgtgaacagcggtgttaccggcaacattccggttgacctgaactttaaccacaacacctacgatagcggtggcgtgatcccggcgagctatctgggtaacgttagcctgcgtaccatcggcagcctgaacattcagaacagcggcgagtggacctacaacggcgtggttcgtgcgtataacgactactatgatttcaacctgggtgattaccgtggcgcgattgcggaaagcctgacctatctgggcgcgcaatttagcggtaagccgtatcacattgcgatgccgggcgaaatcaacattagcggtgcgggtcatcgttaa

SEQ ID NO: 15 - protein, 782 aa, synthetic construct
MSETMVVVAPPTGFEPAGYGGGLFSPSTPNNSPSQGQIFLQVTLPYYQSAKFCQDSMAWLAQYVKTHGATDPLTIQVVANNIRYFLNADTNLCHNPRQNVWEAFHSEMTGSGPAPAKYDYKSMSLKQGSGSASGSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLEVSMGLKLNLTWFDKKTEEFKGGEYSKDFGDDGSVIESLGMPLKDNINNGWFDVEKSWVSILQPHFKNVIDISKFDYFVSFDYRDGNW

SEQ ID NO: 16 - DNA, 2352 bp, synthetic construct
  source 1..2352: mol_type = other DNA
atgagcgagaccatggttgttgttgcgccgccgaccggttttgagccggcgggttacggtggtggcctgttcagcccgagcaccccgaacaacagcccgagccagggtcaaatcttcctgcaagtgaccctgccgtactatcagagcgcgaagttttgccaagacagcatggcgtggctggcgcagtacgttaaaacccacggcgcgaccgatccgctgaccatccaagtggttgcgaacaacattcgttacttcctgaacgcggacaccaacctgtgccacaacccgcgtcagaacgtgtgggaggcgtttcacagcgaaatgaccggtagcggtccggcgccggcgaagtacgattataaaagcatgagcctgaagcaaggctccggatcggcttctgggagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatctttaagaggtaagtatgggacttaaattaaatttaacctggtttgataagaaaaccgaagagtttaaaggtggtgaatactcaaaagacttcggtgatgatggttctgtcattgaaagtctggggatgcctttaaaggataatattaataatggttggtttgatgttgaaaaatcatgggtttcgatattacagccacactttaaaaatgtaatcgatattagtaaatttgattactttgtatcatttgattatcgggatggtaactggtaa

SEQ ID NO: 17 - protein, 747 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKEAAAKEAAAKSETMVVVAPPTGFEPAGYGGGLFSPSTPNNSPSQGQIFLQVTLPYYQSAKFCQDSMAWLAQYVKTHGATDPLTIQVVANNIRYFLNADTNLCHNPRQNVWEAFHSEMTGSGPAPAKYDYKSMSLKQMSGNVVTPAAAFGHYLWGNGEERYVNLPDVGLKITPQQIPELMNIVNSGVTGNIPVDLNFNHNTYDSGGVIPASYLGNVSLRTIGSLNIQNSGEWTYNGVVRAYNDYYDFNLGDYRGAIAESLTYLGAQFSGKPYHIAMPGEINISGAGHR

SEQ ID NO: 18 - DNA, 2244 bp, synthetic construct
  source 1..2244: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaaggaagccgctgccaaggaggcggccgctaaaagcgagaccatggttgttgttgcgccgccgaccggttttgagccggcgggttacggtggtggcctgttcagcccgagcaccccgaacaacagcccgagccagggtcaaatcttcctgcaagtgaccctgccgtactatcagagcgcgaagttttgccaagacagcatggcgtggctggcgcagtacgttaaaacccacggcgcgaccgatccgctgaccatccaagtggttgcgaacaacattcgttacttcctgaacgcggacaccaacctgtgccacaacccgcgtcagaacgtgtgggaggcgtttcacagcgaaatgaccggtagcggtccggcgccggcgaagtacgattataaaagcatgagcctgaagcaaatgagcggtaacgtggttaccccggcggcggcgtttggtcactacctgtggggtaacggcgaggaacgttatgtgaacctgccggacgttggtctgaaaatcaccccgcagcaaattccggagctgatgaacatcgtgaacagcggtgttaccggcaacattccggttgacctgaactttaaccacaacacctacgatagcggtggcgtgatcccggcgagctatctgggtaacgttagcctgcgtaccatcggcagcctgaacattcagaacagcggcgagtggacctacaacggcgtggttcgtgcgtataacgactactatgatttcaacctgggtgattaccgtggcgcgattgcggaaagcctgacctatctgggcgcgcaatttagcggtaagccgtatcacattgcgatgccgggcgaaatcaacattagcggtgcgggtcatcgttaa

SEQ ID NO: 19 - protein, 444 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSGSGSASGSETMVVVAPPTGFEPAGYGGGLFSPSTPNNSPSQGQIFLQVTLPYYQSAKFCQDSMAWLAQYVKTHGATDPLTIQVVANNIRYFLNADTNLCHNPRQNVWEAFHSEMTGSGPAPAKYDYKSMSLKQMSGNVVTPAAAFGHYLWGNGEERYVNLPDVGLKITPQQIPELMNIVNSGVTGNIPVDLNFNHNTYDSGGVIPASYLGNVSLRTIGSLNIQNSGEWTYNGVVRAYNDYYDFNLGDYRGAIAESLTYLGAQFSGKPYHIAMPGEINISGAGHR

SEQ ID NO: 20 - DNA, 1332 bp, synthetic construct
  source 1..1332: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatggctccggatcggcttctgggagcgagaccatggttgttgttgcgccgccgaccggttttgagccggcgggttacggtggtggcctgttcagcccgagcaccccgaacaacagcccgagccagggtcaaatcttcctgcaagtgaccctgccgtactatcagagcgcgaagttttgccaagacagcatggcgtggctggcgcagtacgttaaaacccacggcgcgaccgatccgctgaccatccaagtggttgcgaacaacattcgttacttcctgaacgcggacaccaacctgtgccacaacccgcgtcagaacgtgtgggaggcgtttcacagcgaaatgaccggtagcggtccggcgccggcgaagtacgattataaaagcatgagcctgaagcaaatgagcggtaacgtggttaccccggcggcggcgtttggtcactacctgtggggtaacggcgaggaacgttatgtgaacctgccggacgttggtctgaaaatcaccccgcagcaaattccggagctgatgaacatcgtgaacagcggtgttaccggcaacattccggttgacctgaactttaaccacaacacctacgatagcggtggcgtgatcccggcgagctatctgggtaacgttagcctgcgtaccatcggcagcctgaacattcagaacagcggcgagtggacctacaacggcgtggttcgtgcgtataacgactactatgatttcaacctgggtgattaccgtggcgcgattgcggaaagcctgacctatctgggcgcgcaatttagcggtaagccgtatcacattgcgatgccgggcgaaatcaacattagcggtgcgggtcatcgttaa

SEQ ID NO: 21 - protein, 447 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSEAAAKEAAAKSETMVVVAPPTGFEPAGYGGGLFSPSTPNNSPSQGQIFLQVTLPYYQSAKFCQDSMAWLAQYVKTHGATDPLTIQVVANNIRYFLNADTNLCHNPRQNVWEAFHSEMTGSGPAPAKYDYKSMSLKQMSGNVVTPAAAFGHYLWGNGEERYVNLPDVGLKITPQQIPELMNIVNSGVTGNIPVDLNFNHNTYDSGGVIPASYLGNVSLRTIGSLNIQNSGEWTYNGVVRAYNDYYDFNLGDYRGAIAESLTYLGAQFSGKPYHIAMPGEINISGAGHR

SEQ ID NO: 22 - DNA, 1341 bp, synthetic construct
  source 1..1341: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatgaagccgctgccaaggaggcggccgctaaaagcgagaccatggttgttgttgcgccgccgaccggttttgagccggcgggttacggtggtggcctgttcagcccgagcaccccgaacaacagcccgagccagggtcaaatcttcctgcaagtgaccctgccgtactatcagagcgcgaagttttgccaagacagcatggcgtggctggcgcagtacgttaaaacccacggcgcgaccgatccgctgaccatccaagtggttgcgaacaacattcgttacttcctgaacgcggacaccaacctgtgccacaacccgcgtcagaacgtgtgggaggcgtttcacagcgaaatgaccggtagcggtccggcgccggcgaagtacgattataaaagcatgagcctgaagcaaatgagcggtaacgtggttaccccggcggcggcgtttggtcactacctgtggggtaacggcgaggaacgttatgtgaacctgccggacgttggtctgaaaatcaccccgcagcaaattccggagctgatgaacatcgtgaacagcggtgttaccggcaacattccggttgacctgaactttaaccacaacacctacgatagcggtggcgtgatcccggcgagctatctgggtaacgttagcctgcgtaccatcggcagcctgaacattcagaacagcggcgagtggacctacaacggcgtggttcgtgcgtataacgactactatgatttcaacctgggtgattaccgtggcgcgattgcggaaagcctgacctatctgggcgcgcaatttagcggtaagccgtatcacattgcgatgccgggcgaaatcaacattagcggtgcgggtcatcgttaa

SEQ ID NO: 23 - protein, 696 aa, synthetic construct
MPEETLTVVGGGNNSCNVSWGGGNGNNGGAGYSGKYGGEAAAKEAAAKSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLEVSMGLKLNLTWFDKKTEEFKGGEYSKDFGDDGSVIESLGMPLKDNINNGWFDVEKSWVSILQPHFKNVIDISKFDYFVSFDYRDGNW

SEQ ID NO: 24 - DNA, 2094 bp, synthetic construct
  source 1..2094: mol_type = other DNA
atgcctgaagaaacattgactgtcgtaggtggtggtaataacagctgtaatgttagctggggtggtggcaatggtaacaacggtggtgctggctattctggtaaatacggtggtgaagccgctgccaaggaggcggccgctaaaagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatctttaagaggtaagtatgggacttaaattaaatttaacctggtttgataagaaaaccgaagagtttaaaggtggtgaatactcaaaagacttcggtgatgatggttctgtcattgaaagtctggggatgcctttaaaggataatattaataatggttggtttgatgttgaaaaatcatgggtttcgatattacagccacactttaaaaatgtaatcgatattagtaaatttgattactttgtatcatttgattatcgggatggtaactggtaa

SEQ ID NO: 25 - protein, 664 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFKEAAAKEAAAKSETMVVVAPPTGFEPAGYGGGLFSPSTPNNSPSQGQIFLQVTLPYYQSAKFCQDSMAWLAQYVKTHGATDPLTIQVVANNIRYFLNADTNLCHNPRQNVWEAFHSEMTGSGPAPAKYDYKSMSLKQMSGNVVTPAAAFGHYLWGNGEERYVNLPDVGLKITPQQIPELMNIVNSGVTGNIPVDLNFNHNTYDSGGVIPASYLGNVSLRTIGSLNIQNSGEWTYNGVVRAYNDYYDFNLGDYRGAIAESLTYLGAQFSGKPYHIAMPGEINISGAGHR

SEQ ID NO: 26 - DNA, 1995 bp, synthetic construct
  source 1..1995: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaagaagccgctgccaaggaggcggccgctaaaagcgagaccatggttgttgttgcgccgccgaccggttttgagccggcgggttacggtggtggcctgttcagcccgagcaccccgaacaacagcccgagccagggtcaaatcttcctgcaagtgaccctgccgtactatcagagcgcgaagttttgccaagacagcatggcgtggctggcgcagtacgttaaaacccacggcgcgaccgatccgctgaccatccaagtggttgcgaacaacattcgttacttcctgaacgcggacaccaacctgtgccacaacccgcgtcagaacgtgtgggaggcgtttcacagcgaaatgaccggtagcggtccggcgccggcgaagtacgattataaaagcatgagcctgaagcaaatgagcggtaacgtggttaccccggcggcggcgtttggtcactacctgtggggtaacggcgaggaacgttatgtgaacctgccggacgttggtctgaaaatcaccccgcagcaaattccggagctgatgaacatcgtgaacagcggtgttaccggcaacattccggttgacctgaactttaaccacaacacctacgatagcggtggcgtgatcccggcgagctatctgggtaacgttagcctgcgtaccatcggcagcctgaacattcagaacagcggcgagtggacctacaacggcgtggttcgtgcgtataacgactactatgatttcaacctgggtgattaccgtggcgcgattgcggaaagcctgacctatctgggcgcgcaatttagcggtaagccgtatcacattgcgatgccgggcgaaatcaacattagcggtgcgggtcatcgttaa

SEQ ID NO: 27 - protein, 661 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFKGSGSASGSETMVVVAPPTGFEPAGYGGGLFSPSTPNNSPSQGQIFLQVTLPYYQSAKFCQDSMAWLAQYVKTHGATDPLTIQVVANNIRYFLNADTNLCHNPRQNVWEAFHSEMTGSGPAPAKYDYKSMSLKQMSGNVVTPAAAFGHYLWGNGEERYVNLPDVGLKITPQQIPELMNIVNSGVTGNIPVDLNFNHNTYDSGGVIPASYLGNVSLRTIGSLNIQNSGEWTYNGVVRAYNDYYDFNLGDYRGAIAESLTYLGAQFSGKPYHIAMPGEINISGAGHR

SEQ ID NO: 28 - DNA, 1986 bp, synthetic construct
  source 1..1986: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaaggctccggatcggcttctgggagcgagaccatggttgttgttgcgccgccgaccggttttgagccggcgggttacggtggtggcctgttcagcccgagcaccccgaacaacagcccgagccagggtcaaatcttcctgcaagtgaccctgccgtactatcagagcgcgaagttttgccaagacagcatggcgtggctggcgcagtacgttaaaacccacggcgcgaccgatccgctgaccatccaagtggttgcgaacaacattcgttacttcctgaacgcggacaccaacctgtgccacaacccgcgtcagaacgtgtgggaggcgtttcacagcgaaatgaccggtagcggtccggcgccggcgaagtacgattataaaagcatgagcctgaagcaaatgagcggtaacgtggttaccccggcggcggcgtttggtcactacctgtggggtaacggcgaggaacgttatgtgaacctgccggacgttggtctgaaaatcaccccgcagcaaattccggagctgatgaacatcgtgaacagcggtgttaccggcaacattccggttgacctgaactttaaccacaacacctacgatagcggtggcgtgatcccggcgagctatctgggtaacgttagcctgcgtaccatcggcagcctgaacattcagaacagcggcgagtggacctacaacggcgtggttcgtgcgtataacgactactatgatttcaacctgggtgattaccgtggcgcgattgcggaaagcctgacctatctgggcgcgcaatttagcggtaagccgtatcacattgcgatgccgggcgaaatcaacattagcggtgcgggtcatcgttaa

SEQ ID NO: 29 - protein, 643 aa, synthetic construct
MGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLEVSMGLKLNLTWFDKKTEEFKGGEYSKDFGDDGSVIESLGMPLKDNINNGWFDVEKSWVSILQPHFKNVIDISKFDYFVSFDYRDGNW

SEQ ID NO: 30 - DNA, 1935 bp, synthetic construct
  source 1..1935: mol_type = other DNA
atgggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatctttaagaggtaagtatgggacttaaattaaatttaacctggtttgataagaaaaccgaagagtttaaaggtggtgaatactcaaaagacttcggtgatgatggttctgtcattgaaagtctggggatgcctttaaaggataatattaataatggttggtttgatgttgaaaaatcatgggtttcgatattacagccacactttaaaaatgtaatcgatattagtaaatttgattactttgtatcatttgattatcgggatggtaactggtaa

SEQ ID NO: 31 - protein, 625 aa, synthetic construct
MNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLEVSMGLKLNLTWFDKKTEEFKGGEYSKDFGDDGSVIESLGMPLKDNINNGWFDVEKSWVSILQPHFKNVIDISKFDYFVSFDYRDGNW

SEQ ID NO: 32 - DNA, 1881 bp, synthetic construct
  source 1..1881: mol_type = other DNA
atgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatctttaagaggtaagtatgggacttaaattaaatttaacctggtttgataagaaaaccgaagagtttaaaggtggtgaatactcaaaagacttcggtgatgatggttctgtcattgaaagtctggggatgcctttaaaggataatattaataatggttggtttgatgttgaaaaatcatgggtttcgatattacagccacactttaaaaatgtaatcgatattagtaaatttgattactttgtatcatttgattatcgggatggtaactggtaa

SEQ ID NO: 33 - protein, 470 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSQGQIFLQVTLPYYQSAKFCQDSMAWLAQYVKTHGATDPLTIQVVANNIRYFLNADTNLCHNPRQNVWEAFHSEMTGSGPAPAKYDYKSMSLKQMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFK

SEQ ID NO: 34 - DNA, 1413 bp, synthetic construct
  source 1..1413: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagccagggtcaaatcttcctgcaagtgaccctgccgtactatcagagcgcgaagttttgccaagacagcatggcgtggctggcgcagtacgttaaaacccacggcgcgaccgatccgctgaccatccaagtggttgcgaacaacattcgttacttcctgaacgcggacaccaacctgtgccacaacccgcgtcagaacgtgtgggaggcgtttcacagcgaaatgaccggtagcggtccggcgccggcgaagtacgattataaaagcatgagcctgaagcaaatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaataa

SEQ ID NO: 35 - protein, 687 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKEAAAKEAAAKMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFK

SEQ ID NO: 36 - DNA, 2064 bp, synthetic construct
  source 1..2064: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaaggaagccgctgccaaggaggcggccgctaaaatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaataa

SEQ ID NO: 37 - protein, 348 aa, synthetic construct
MPEETLTVVGGGNNSCNVSWGGGNGNNGGAGYSGKYGGTPSQGQIFLQVTLPYYQSAKFCQDSMAWLAQYVKTHGATDPLTIQVVANNIRYFLNADTNLCHNPRQNVWEAFHSEMTGSGPAPAKYDYKSMSLKLQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFK

SEQ ID NO: 38 - DNA, 1047 bp, synthetic construct
  source 1..1047: mol_type = other DNA
atgcctgaagaaacattgactgtcgtaggtggtggtaataacagctgtaatgttagctggggtggtggcaatggtaacaacggtggtgctggctattctggtaaatacggtggtaccccgagccagggtcaaatcttcctgcaagtgaccctgccgtactatcagagcgcgaagttttgccaagacagcatggcgtggctggcgcagtacgttaaaacccacggcgcgaccgatccgctgaccatccaagtggttgcgaacaacattcgttacttcctgaacgcggacaccaacctgtgccacaacccgcgtcagaacgtgtgggaggcgtttcacagcgaaatgaccggtagcggtccggcgccggcgaagtacgattataaaagcatgagcctgaagcttcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaataa

SEQ ID NO: 39 - protein, 307 aa, synthetic construct
MSETMVVVAPPTGFEPAGYGGGLFSPSTPNGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKLMSGNVVTPAAAFGHYLWGNGEERYVNLPDVGLKITPQQIPELMNIVNSGVTGNIPVDLNFNHNTYDSGGVIPASYLGNVSLRTIGSLNIQNSGEWTYNGVVRAYNDYYDFNLGDYRGAIAESLTYLGAQFSGKPYHIAMPGEINISGAGHR

SEQ ID NO: 40 - DNA, 924 bp, synthetic construct
  source 1..924: mol_type = other DNA
atgagcgagaccatggttgttgttgcgccgccgaccggttttgagccggcgggttacggtggtggcctgttcagcccgagcaccccgaacggtaccagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaagcttatgagcggtaacgtggttaccccggcggcggcgtttggtcactacctgtggggtaacggcgaggaacgttatgtgaacctgccggacgttggtctgaaaatcaccccgcagcaaattccggagctgatgaacatcgtgaacagcggtgttaccggcaacattccggttgacctgaactttaaccacaacacctacgatagcggtggcgtgatcccggcgagctatctgggtaacgttagcctgcgtaccatcggcagcctgaacattcagaacagcggcgagtggacctacaacggcgtggttcgtgcgtataacgactactatgatttcaacctgggtgattaccgtggcgcgattgcggaaagcctgacctatctgggcgcgcaatttagcggtaagccgtatcacattgcgatgccgggcgaaatcaacattagcggtgcgggtcatcgttaa

SEQ ID NO: 41 - protein, 793 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHEAAAKEAAAKNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLEVSMGLKLNLTWFDKKTEEFKGGEYSKDFGDDGSVIESLGMPLKDNINNGWFDVEKSWVSILQPHFKNVIDISKFDYFVSFDYRDGNW

SEQ ID NO: 42 - DNA, 2385 bp, synthetic construct
  source 1..2385: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatgaagccgctgccaaggaggcggccgctaaaaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatctttaagaggtaagtatgggacttaaattaaatttaacctggtttgataagaaaaccgaagagtttaaaggtggtgaatactcaaaagacttcggtgatgatggttctgtcattgaaagtctggggatgcctttaaaggataatattaataatggttggtttgatgttgaaaaatcatgggtttcgatattacagccacactttaaaaatgtaatcgatattagtaaatttgattactttgtatcatttgattatcgggatggtaactggtaa

SEQ ID NO: 43 - protein, 382 aa, synthetic construct
MSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSEAAAKEAAAKMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFK

SEQ ID NO: 44 - DNA, 1149 bp, synthetic construct
  source 1..1149: mol_type = other DNA
atgtcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgaagccgctgccaaggaggcggccgctaaaatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaataa

SEQ ID NO: 45 - protein, 745 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKEAAAKEAAAKSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

SEQ ID NO: 46 - DNA, 2238 bp, synthetic construct
  source 1..2238: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaaggaagccgctgccaaggaggcggccgctaaatcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgttggtctgaagatccaggcaaaccagatcgacccggttatgattgcggttaaaaacaacgcagcaggcacctaccagatctccggcaacttcaaccgtaatacctttatcgacggcgatattccgggtttatatctgggtaacattaccatgaaaacggaaggcacactcaagatcgacgccaagggtaattggaattataacggtgtcgtgcgcgcgtttaacgatacctacgacgctaatccgtcgacccaccgtagcaaaagcgcagaagatctgacgacgttgctgcgtttgacgcaaggtacgccgtacgagatcagaatcccgggtgagctgaaggtgagcggtagcggcaagaagtaa

SEQ ID NO: 47 - protein, 742 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKGSGSASGSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

SEQ ID NO: 48 - DNA, 2229 bp, synthetic construct
  source 1..2229: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagggctccggatcggcttctgggtcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgttggtctgaagatccaggcaaaccagatcgacccggttatgattgcggttaaaaacaacgcagcaggcacctaccagatctccggcaacttcaaccgtaatacctttatcgacggcgatattccgggtttatatctgggtaacattaccatgaaaacggaaggcacactcaagatcgacgccaagggtaattggaattataacggtgtcgtgcgcgcgtttaacgatacctacgacgctaatccgtcgacccaccgtagcaaaagcgcagaagatctgacgacgttgctgcgtttgacgcaaggtacgccgtacgagatcagaatcccgggtgagctgaaggtgagcggtagcggcaagaagtaa

SEQ ID NO: 49 - protein, 735 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

SEQ ID NO: 50 - DNA, 2208 bp, synthetic construct
  source 1..2208: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagtcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgttggtctgaagatccaggcaaaccagatcgacccggttatgattgcggttaaaaacaacgcagcaggcacctaccagatctccggcaacttcaaccgtaatacctttatcgacggcgatattccgggtttatatctgggtaacattaccatgaaaacggaaggcacactcaagatcgacgccaagggtaattggaattataacggtgtcgtgcgcgcgtttaacgatacctacgacgctaatccgtcgacccaccgtagcaaaagcgcagaagatctgacgacgttgctgcgtttgacgcaaggtacgccgtacgagatcagaatcccgggtgagctgaaggtgagcggtagcggcaagaagtaa

SEQ ID NO: 51 - protein, 755 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKEAAAKEAAAKEAAAKEAAAKSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

SEQ ID NO: 52 - DNA, 2268 bp, synthetic construct
  source 1..2268: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaaggaagccgctgccaaggaggcggccgctaaagaagccgctgccaaggaggcggccgctaaatcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgttggtctgaagatccaggcaaaccagatcgacccggttatgattgcggttaaaaacaacgcagcaggcacctaccagatctccggcaacttcaaccgtaatacctttatcgacggcgatattccgggtttatatctgggtaacattaccatgaaaacggaaggcacactcaagatcgacgccaagggtaattggaattataacggtgtcgtgcgcgcgtttaacgatacctacgacgctaatccgtcgacccaccgtagcaaaagcgcagaagatctgacgacgttgctgcgtttgacgcaaggtacgccgtacgagatcagaatcccgggtgagctgaaggtgagcggtagcggcaagaagtaa

SEQ ID NO: 53 - protein, 742 aa, synthetic construct; identical to SEQ ID NO: 47

SEQ ID NO: 54 - DNA, 2229 bp, synthetic construct; identical to SEQ ID NO: 48
  source 1..2229: mol_type = other DNA

SEQ ID NO: 55 - protein, 836 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

SEQ ID NO: 56 - DNA, 2532 bp, synthetic construct
  source 1..2532: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatcttggctccggatcggcttctgggtcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgttggtctgaagatccaggcaaaccagatcgacccggttatgattgcggttaaaaacaacgcagcaggcacctaccagatctccggcaacttcaaccgtaatacctttatcgacggcgatattccgggtttatatctgggtaacattaccatgaaaacggaaggcacactcaagatcgacgccaagggtaattggaattataacggtgtcgtgcgcgcgtttaacgatacctacgacgctaatccgtcgacccaccgtagcaaaagcgcagaagatctgacgacgttgctgcgtttgacgcaaggtacgccgtacgagatcagaatcccgggtgagctgaaggtgagcggtagcggcaagaagtaa

SEQ ID NO: 57 - protein, 843 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLGSGSASGSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

SEQ ID NO: 58 - DNA, 2532 bp, synthetic construct; identical to SEQ ID NO: 56
  source 1..2532: mol_type = other DNA

SEQ ID NO: 59 - protein, 846 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPKMMSKIVTSLPADTVTETPVSSLPLDQATVSVTKRVADIVKDERQHIAVVTGRPMSVPVVDAKPTKRPGVFSVSIPGLPSLQVSVPKGVPAAKAPPKGIIAEKGDSRPAGFTAGGNSREAVIRFPKESGQKPVYVSVTDVLTPAQVKQRLEEEKRRQQAWDAAHPEEGLKREYDKAKAELDAEDKNIATLNSRIASTEKAIPGARAAVQEADKKVKEAEANKDDFVTYNPPHEYGSGWQDQVRYLDKDIQNQNEKLKAAQTSLNEMNESLSRDKAALSGAMESRKQKEKKAKDAENKLNEEKKKPRKGTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYLEAAAKEAAAKSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

SEQ ID NO: 60 - DNA, 2541 bp, synthetic construct
  source 1..2541: mol_type = other DNA
atgagtggtggagacggacgaggtccgggtaattcaggtctgggacacaatggtggtcaggccagtgggaatgtgaacggtacatcaggtaaaggtggcccttcatcaggtggtggtacggatccaaacagcgggccgggctggggtacgacgcatacacctaacggggatattcataactataacccgggggagtttggtaacggcgggagtaaacccggtggtaatggcggtaacagcggcaatcatagcgggagctctggtggcggacagtcttcggccaccgcgatggccttcggtctgcctgccttggccactccgggggctgaaggactggctttatccgtttccggcgatgcgttgtcggccgctgttgctgatgtgctggctgccctgaaaggtccgtttaagtttggtctgtgggggattgcgatttacggagtgctgccttctgagatagcaaaagatgatccgaaaatgatgtcaaaaatcgtgacgtcattgccggcagacacggtaacggagacgccggtcagctccctgccgctggaccaggcgacggtcagcgtcactaaacgtgtggcggatattgtgaaggacgagcggcagcatattgcggttgtcaccggccggccaatgagtgttcctgtggtggatgcgaaaccgacaaaacgtccgggggtattcagtgtgtcgattccgggtctcccgtctctgcaggtgagcgtacctaaaggtgttccggccgcgaaagccccgccaaaaggcattattgctgaaaaaggtgattcacgtccggctggttttacggccggtggtaactcccgtgaagccgttattcgtttcccgaaagagagcggacagaaaccggtctatgtgtcggtgacggatgttcttactccggcacaggtaaaacagcgtctggaggaagaaaagcgtcgccagcaggcatgggacgctgctcacccggaagaggggctgaaaagagagtatgataaagcgaaagctgagctggatgctgaagataaaaatattgcgaccttaaacagtcgcattgcatcgacagagaaggcgatccccggtgcaagggctgctgttcaggaagccgataaaaaggtaaaagaggcagaggcaaataaagatgattttgtgacttataacccgcctcatgaatatggctccgggtggcaggatcaggttcgctatcttgataaggatattcagaatcagaatgagaaattaaaagctgcccagacatctttaaacgaaatgaatgaatccttatccagggataaggctgcgctttccggggcgatggagagccggaaacaaaaggagaaaaaagcgaaggatgcagaaaataaattaaatgaggaaaagaaaaaacctcgtaagggaactaaagattacggccatgattattttcctgatcccaagactgaagatattaaggggttgggagagttgaaagagggtaaacctaaaacccctaaacaaggtggtggtggtaagcgggcccgatggtatggtgataaaaagcgtaaaatttatgaatgggattcccagcacggtgagcttgaagggtaccgcgccagtgatggcgaacacctcggggcattcgatccaaaaacgggtaagcaggttaaagggccggatccaaaacgaaacattaaaaaatatcttgaagccgctgccaaggaggcggccgctaaatcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgttggtctgaagatccaggcaaaccagatcgacccggttatgattgcggttaaaaacaacgcagcaggcacctaccagatctccggcaacttcaaccgtaatacctttatcgacggcgatattccgggtttatatctgggtaacattaccatgaaaacggaaggcacactcaagatcgacgccaagggtaattggaattataacggtgtcgtgcgcgcgtttaacgatacctacgacgctaatccgtcgacccaccgtagcaaaagcgcagaagatctgacgacgttgctgcgtttgacgcaaggtacgccgtacgagatcagaatcccgggtgagctgaaggtgagcggtagcggcaagaagtaa

SEQ ID NO: 61 - protein, 662 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFKEAAAKEAAAKSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

SEQ ID NO: 62 - DNA, 1989 bp, synthetic construct
  source 1..1989: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaagaagccgctgccaaggaggcggccgctaaatcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgttggtctgaagatccaggcaaaccagatcgacccggttatgattgcggttaaaaacaacgcagcaggcacctaccagatctccggcaacttcaaccgtaatacctttatcgacggcgatattccgggtttatatctgggtaacattaccatgaaaacggaaggcacactcaagatcgacgccaagggtaattggaattataacggtgtcgtgcgcgcgtttaacgatacctacgacgctaatccgtcgacccaccgtagcaaaagcgcagaagatctgacgacgttgctgcgtttgacgcaaggtacgccgtacgagatcagaatcccgggtgagctgaaggtgagcggtagcggcaagaagtaa

SEQ ID NO: 63 - protein, 659 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFKGSGSASGSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

SEQ ID NO: 64 - DNA, 1980 bp, synthetic construct
  source 1..1980: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaaggctccggatcggcttctgggtcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgttggtctgaagatccaggcaaaccagatcgacccggttatgattgcggttaaaaacaacgcagcaggcacctaccagatctccggcaacttcaaccgtaatacctttatcgacggcgatattccgggtttatatctgggtaacattaccatgaaaacggaaggcacactcaagatcgacgccaagggtaattggaattataacggtgtcgtgcgcgcgtttaacgatacctacgacgctaatccgtcgacccaccgtagcaaaagcgcagaagatctgacgacgttgctgcgtttgacgcaaggtacgccgtacgagatcagaatcccgggtgagctgaaggtgagcggtagcggcaagaagtaa

SEQ ID NO: 65 - protein, 672 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFKEAAAKEAAAKEAAAKEAAAKSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

SEQ ID NO: 66 - DNA, 2019 bp, synthetic construct
  source 1..2019: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaagaagccgctgccaaggaggcggccgctaaagaagccgctgccaaggaggcggccgctaaatcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgttggtctgaagatccaggcaaaccagatcgacccggttatgattgcggttaaaaacaacgcagcaggcacctaccagatctccggcaacttcaaccgtaatacctttatcgacggcgatattccgggtttatatctgggtaacattaccatgaaaacggaaggcacactcaagatcgacgccaagggtaattggaattataacggtgtcgtgcgcgcgtttaacgatacctacgacgctaatccgtcgacccaccgtagcaaaagcgcagaagatctgacgacgttgctgcgtttgacgcaaggtacgccgtacgagatcagaatcccgggtgagctgaaggtgagcggtagcggcaagaagtaa

SEQ ID NO: 67 - protein, 275 aa, synthetic construct
MSDTMIVVATPTPGFSYASGLTYGGGMFSPTISGPSEGQIFLQVTLPYYQSEKFCADQLNWLVQYIKKHGASDPMTIQVAANNIRYLCNADTNLVKNPKTLVYDAFHSRSPAPANYDYRSMNLKQMSGSVVTPGAAFAHYLWGNGETRFVNLPDVGLKVTPQQIPELMKLVNSGVTGAIPVSVKFSRDTSTDSITAGAYLGHITLQTEGTLNVTGNGAWTYNGVIRAYDDTYDFNLGNFRGPIAESLTFLGSTFSGKPYQISLPGQINISGSGKK

SEQ ID NO: 68 - DNA, 828 bp, synthetic construct
  source 1..828: mol_type = other DNA
atgtcagatacaatgatagtagttgctactccgaccccgggttttagctacgcgagcggcttgacgtacggtggcggtatgtttagcccgaccatcagcggtccgagcgagggtcagatttttctgcaagtgaccctgccgtactatcagagcgaaaagttctgcgcggatcagctgaactggctggtgcaatatattaagaaacacggcgcgagcgacccgatgaccatccaagttgcggcgaacaacattcgttacctgtgcaacgcggacaccaacctggtgaagaacccgaaaaccctggtttatgatgcgttccacagccgtagcccggcgccggcgaactacgactatcgtagcatgaacctgaagcagatgagcggcagcgtggttacgccgggtgcggcgtttgcgcactacctgtggggcaacggcgagacccgttttgttaacctgccggatgtgggtctgaaggttaccccgcagcaaatcccggaactgatgaaactggtgaacagcggcgttaccggtgcgattccggtgagcgttaaattcagccgtgacaccagcaccgatagcatcaccgcgggcgcgtacctgggtcacattaccctgcaaaccgagggcaccctgaacgtgaccggcaacggtgcgtggacctataacggtgttatccgtgcgtacgacgatacctatgacttcaacctgggcaactttcgtggtccgattgcggaaagcctgaccttcctgggcagcacctttagcggcaagccgtaccagatcagcctgccgggtcaaatcaacattagcggcagcggtaaaaagtaa

SEQ ID NO: 69 - protein, 502 aa, synthetic construct
MSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKKEAAAKEAAAKGVALDRTRVDPQAVGNEVLKRNADKLNAMRGAEYGANVKVSGTDIRMNGGNSAGMLKQDVFNWRKELAQFEAYRGEAYKDADGYSVGLGHYLGSGNAGAGTTVTPEQAAQWFAEDTDRALDQGVRLADELGVTNNASILGLAGMAFQMGEGRARQFRNTFQAIKDRNKEAFEAGVRNSKWYTQTPNRAEAFIKRMAPHFDTPSQIGVDWYSAATAE

SEQ ID NO: 70 - DNA, 1509 bp, synthetic construct
  source 1..1509: mol_type = other DNA
atgtcagatacaatgatagtagttgctactccgaccccgggttttagctacgcgagcggcttgacgtacggcggtggtgcgtttgcgggtgcgcctgcgaacggtccgtcggaaggccaaatctttttccagaccgttctgccggcgtatcaatccccgaatctgtgtattggtcaactggcttggatgaccgattacattaataagaacggggtgggtaacccaaaaacctgggaagttatttcccaaaacgttctgattttctgcagcgcggatactgccctggtgcttaatccgcgcatcgcggtgtacgacggcttccacaaaactaaatgggcgccagccaagttcaattttaaaacccagagccaggagaaattctctggcaacgtgaccaccccgattgctgccttcggccattatctgtggggtgagggcaagccgcgtaccgtcgacttgtctagcgttggtctgaagatccaggcaaaccagatcgacccggttatgattgcggttaaaaacaacgcagcaggcacctaccagatctccggcaacttcaaccgtaatacctttatcgacggcgatattccgggtttatatctgggtaacattaccatgaaaacggaaggcacactcaagatcgacgccaagggtaattggaattataacggtgtcgtgcgcgcgtttaacgatacctacgacgctaatccgtcgacccaccgtagcaaaagcgcagaagatctgacgacgttgctgcgtttgacgcaaggtacgccgtacgagatcagaatcccgggtgagctgaaggtgagcggtagcggcaagaaggaagccgctgccaaggaggcggccgctaaaggtgtggccctggaccgcacgcgggttgatccccaggcagtcggcaacgaggtgctcaagcgcaacgcggataagctgaatgcgatgcggggcgccgagtacggtgccaacgtcaaggtcagcggcacggacattcgcatgaacgggggtaacagtgccggcatgctgaagcaggacgtgttcaactggcggaaggaactggctcagttcgaggcttaccgaggggaggcgtataaggatgccgatggttatagtgtgggcctggggcattacctgggcagtggcaatgctggggcaggtactacagtcacgcctgagcaagccgcgcagtggttcgccgaggacaccgaccgcgcactcgaccagggtgtgaggttggccgacgagctgggcgttacgaacaatgcctctatcctgggattggccggtatggccttccagatgggcgaaggacgtgcccggcagttccgtaacaccttccaggcgatcaaggatcgcaacaaggaagccttcgaggctggtgtgcgaaacagcaagtggtacacgcagacgcccaaccgggccgaggcattcatcaagcgcatggcgccccacttcgatacaccgagtcaaatcggtgtcgattggtacagcgccgcaacagcggagtga

SEQ ID NO: 71 - protein, 593 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFKGVALDRTRVDPQAVGNEVLKRNADKLNAMRGAEYGANVKVSGTDIRMNGGNSAGMLKQDVFNWRKELAQFEAYRGEAYKDADGYSVGLGHYLGSGNAGAGTTVTPEQAAQWFAEDTDRALDQGVRLADELGVTNNASILGLAGMAFQMGEGRARQFRNTFQAIKDRNKEAFEAGVRNSKWYTQTPNRAEAFIKRMAPHFDTPSQIGVDWYSAATAE

SEQ ID NO: 72 - DNA, 1782 bp, synthetic construct
  source 1..1782: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaaggtgtggccctggaccgcacgcgggttgatccccaggcagtcggcaacgaggtgctcaagcgcaacgcggataagctgaatgcgatgcggggcgccgagtacggtgccaacgtcaaggtcagcggcacggacattcgcatgaacgggggtaacagtgccggcatgctgaagcaggacgtgttcaactggcggaaggaactggctcagttcgaggcttaccgaggggaggcgtataaggatgccgatggttatagtgtgggcctggggcattacctgggcagtggcaatgctggggcaggtactacagtcacgcctgagcaagccgcgcagtggttcgccgaggacaccgaccgcgcactcgaccagggtgtgaggttggccgacgagctgggcgttacgaacaatgcctctatcctgggattggccggtatggccttccagatgggcgaaggacgtgcccggcagttccgtaacaccttccaggcgatcaaggatcgcaacaaggaagccttcgaggctggtgtgcgaaacagcaagtggtacacgcagacgcccaaccgggccgaggcattcatcaagcgcatggcgccccacttcgatacaccgagtcaaatcggtgtcgattggtacagcgccgcaacagcggagtga

SEQ ID NO: 73 - protein, 600 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFKGSGSASGGVALDRTRVDPQAVGNEVLKRNADKLNAMRGAEYGANVKVSGTDIRMNGGNSAGMLKQDVFNWRKELAQFEAYRGEAYKDADGYSVGLGHYLGSGNAGAGTTVTPEQAAQWFAEDTDRALDQGVRLADELGVTNNASILGLAGMAFQMGEGRARQFRNTFQAIKDRNKEAFEAGVRNSKWYTQTPNRAEAFIKRMAPHFDTPSQIGVDWYSAATAE

SEQ ID NO: 74 - DNA, 1803 bp, synthetic construct
  source 1..1803: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaaggctccggatcggcttctgggggtgtggccctggaccgcacgcgggttgatccccaggcagtcggcaacgaggtgctcaagcgcaacgcggataagctgaatgcgatgcggggcgccgagtacggtgccaacgtcaaggtcagcggcacggacattcgcatgaacgggggtaacagtgccggcatgctgaagcaggacgtgttcaactggcggaaggaactggctcagttcgaggcttaccgaggggaggcgtataaggatgccgatggttatagtgtgggcctggggcattacctgggcagtggcaatgctggggcaggtactacagtcacgcctgagcaagccgcgcagtggttcgccgaggacaccgaccgcgcactcgaccagggtgtgaggttggccgacgagctgggcgttacgaacaatgcctctatcctgggattggccggtatggccttccagatgggcgaaggacgtgcccggcagttccgtaacaccttccaggcgatcaaggatcgcaacaaggaagccttcgaggctggtgtgcgaaacagcaagtggtacacgcagacgcccaaccgggccgaggcattcatcaagcgcatggcgccccacttcgatacaccgagtcaaatcggtgtcgattggtacagcgccgcaacagcggagtga

SEQ ID NO: 75 - protein, 603 aa, synthetic construct
MPEETLTVVGGGNNSCNVSWGGGNGNNGGAGYSGKYGGTSYEGATSMLKLNDRVLIQLYLCNPLNPDYIGAPWGSDKDAESIIRANRDKAGKFKANIQNWKTSGTGSLGSPVVGKSYSSGDVDTYSVSFGKEKYNVLYNRKKDSFTTAYVDGGANKPEHSMKDQAIAVVKLYLLNESQASVIDTTSGIITDSGKTLSGKLGDKYNTLAKEAADNIKNFQGKKLRSFNDAMASINELANNPKMKLSQADKTVVSNALKQMDLSALADRFKGLEKAFTWGDRLLKAEKIREGVVTGITTGDWQKLAFEVEAMYLSGVAGAVALGITTAMISTVAVALSLPAVAVSALTVVSVIGISILTSYIDADKAKALNNAVLGLFKEAAAKEAAAKGVALDRTRVDPQAVGNEVLKRNADKLNAMRGAEYGANVKVSGTDIRMNGGNSAGMLKQDVFNWRKELAQFEAYRGEAYKDADGYSVGLGHYLGSGNAGAGTTVTPEQAAQWFAEDTDRALDQGVRLADELGVTNNASILGLAGMAFQMGEGRARQFRNTFQAIKDRNKEAFEAGVRNSKWYTQTPNRAEAFIKRMAPHFDTPSQIGVDWYSAATAE

SEQ ID NO: 76 - DNA, 1812 bp, synthetic construct
  source 1..1812: mol_type = other DNA
atgcctgaagaaacattgactgtcgtaggtggtggtaataacagctgtaatgttagctggggtggtggcaatggtaacaacggtggtgctggctattctggtaaatacggtggtactagctatgaaggtgcaactagtatgttgaagttgaatgaccgtgttttaattcagctttatctttgcaatccgcttaacccagattatattggagcaccttggggcagtgataaagatgcagaatcaattatcagagctaacagagataaagcaggaaagttcaaggctaatattcagaactggaaaacaagcggtacaggttctttaggtagccctgtagttgggaaatcttatagctcaggtgatgtagatacttattcggttagcttcggaaaagaaaaatacaatgtcctgtataaccgtaaaaaagactcttttactacagcttatgtcgatggcggcgcaaataaacctgagcatagcatgaaggatcaggctattgccgttgttaaactttaccttctcaacgaaagtcaggcatcggtaatcgatactacatcgggaattattactgactctggtaaaactcttagcgggaaattaggtgataaatacaacactctggcgaaagaagctgctgacaatataaaaaacttccagggtaagaaactccgcagttttaatgatgctatggcatctattaatgaactagctaacaatccaaagatgaagttaagtcaggcggataaaacagtcgtttctaatgccctcaaacaaatggatttgtcagcactagctgaccgattcaaagggttagagaaagcctttacttggggtgatcgacttcttaaagccgagaaaatcagagaaggtgttgttactggtattaccacaggggactggcaaaagctggcgtttgaggttgaagctatgtacctcagtggtgttgctggcgccgtagcgttagggattactactgccatgattagcacagtcgcagtcgctttgtcacttccagctgtagctgtctctgcgcttactgttgtgtccgtcattggcatctctattctcacatcttatatcgatgctgataaggccaaagcactgaataatgcagtgcttggcttatttaaagaagccgctgccaaggaggcggccgctaaaggtgtggccctggaccgcacgcgggttgatccccaggcagtcggcaacgaggtgctcaagcgcaacgcggataagctgaatgcgatgcggggcgccgagtacggtgccaacgtcaaggtcagcggcacggacattcgcatgaacgggggtaacagtgccggcatgctgaagcaggacgtgttcaactggcggaaggaactggctcagttcgaggcttaccgaggggaggcgtataaggatgccgatggttatagtgtgggcctggggcattacctgggcagtggcaatgctggggcaggtactacagtcacgcctgagcaagccgcgcagtggttcgccgaggacaccgaccgcgcactcgaccagggtgtgaggttggccgacgagctgggcgttacgaacaatgcctctatcctgggattggccggtatggccttccagatgggcgaaggacgtgcccggcagttccgtaacaccttccaggcgatcaaggatcgcaacaaggaagccttcgaggctggtgtgcgaaacagcaagtggtacacgcagacgcccaaccgggccgaggcattcatcaagcgcatggcgccccacttcgatacaccgagtcaaatcggtgtcgattggtacagcgccgcaacagcggagtga

SEQ ID NO: 77 - protein, 6 aa, synthetic construct
HHHHHH